(19) United States
(12) Patent Application Publication
(10) Pub. No.: US 2022/0306623 A1
(43) Pub. Date: Sep. 29, 2022

(54) PYRIDYL OR PYRIMIDYL MTOR KINASE INHIBITORS

(71) Applicant: GLAXOSMITHKLINE INTELLECTUAL PROPERTY DEVELOPMENT LIMITED, Brentford, Middlesex (GB)

(72) Inventors: Sophie Marie BERTRAND, Stevenage (GB); Heather HOBBS, Stevenage (GB); Simon Marc NICOLLE, Stevenage (GB); Sandeep PAL, Stevenage (GB); Simon PEACE, Stevenage (GB); Margarita PUENTE-FELIPE, Madrid (ES)

(21) Appl. No.: 17/617,898

(22) PCT Filed: Jun. 11, 2020
(86) PCT No.: PCT/EP2020/066155

§ 371 Date: Dec. 9, 2021

(30) Foreign Application Priority Data
Jun. 13, 2019 (GB) 1908536.4

(51) Int. Cl. C07D 471/04

(52) U.S. Cl. CPC C07D 471/04

(57) ABSTRACT

The invention relates to compounds or pharmaceutically acceptable salts thereof of formula (I):

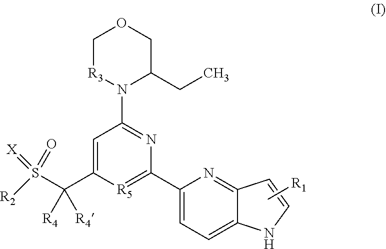

wherein R₁, R₂, R₃, R₄, R_{4′} and R₅ are as defined in the description and claims; and compounds or pharmaceutically acceptable salts thereof of formulas (II), (IIa), (IIb), (IIc), and (III) having mTOR kinase inhibitor activity. The invention also relates to pharmaceutical compositions which include a compound of formula (I), (II), (IIa), (IIb), (IIc), or (III) or a pharmaceutically acceptable salt thereof, and to the use of a compound of formula (I), (II), (IIa), (IIb), (IIc), or (III), or a pharmaceutically acceptable salt thereof in therapy, including in the treatment of a disease or condition for which an mTOR kinase inhibitor activity is indicated, and in particular the treatment of idiopathic pulmonary fibrosis.

### FIELD OF THE INVENTION

The present invention is directed to mTOR kinase inhibitors. The present invention is also directed to pharmaceutical compositions thereof and the use of the compounds and compositions in therapy.

### BACKGROUND TO THE INVENTION

The mammalian target of rapamycin (mTOR) is an evolutionarily conserved serine/threonine kinase and functionally regulates a diverse range of cellular activities. The target of rapamycin (TOR), mammalian TOR (mTOR), also known as the FKBP-12-rapamycin associated protein (FRAP) or rapamycin and FKBP target (RAFT)1 and rapamycin target (RAPT) controls diverse cellular processes ranging from protein translation in response to amino acids or growth factors, autophagy, metabolism, inflammation, lipid synthesis and cytoskeletal rearrangements. Within the cell, mTOR exists as two distinct protein complexes known as mTORC1 (mTOR, RAPTOR, mLST8, PRAS40 and DEPTOR) and mTORC2 (mTOR, RICTOR, mLST8, mSIN1 and PROTOR) each complex consisting of the protein kinase domain, mTOR and complex specific accessory proteins. Both complexes share two common components; mLST8 and Deptor but other components are distinct. mTORC1 uniquely consists of PRAS40 and Raptor whilst mTORC2 requires Rictor, Protor and Sin1. mLST8, Raptor, Rictor and Sin1 are critical for complex assembly and/or link mTOR kinase to its substrate.

Upstream and downstream effectors of mTORC1 have been characterised much more extensively than for mTORC2. mTORC1 is activated by insulin, amino acids and repressed by AMP-activated protein kinase (AMPK). mTORC1 can promote mRNA translation and protein synthesis via two substrates; ribosomal protein S6 kinases (S6Ks) and eukaryotic translation initiation factor 4E-binding protein (4E-BP)1. In addition, mTORC1 represses autophagy, regulates glucose metabolism and mitochondrial function. mTORC1 has been confirmed as a central regulator of longevity as the allosteric mTORC1 inhibitor, rapamycin has been shown to extend lifespan in yeast, nematodes, fruit flies and mice (reviewed in Johnson et al, 2013).

The downstream substrates and signalling pathways for mTORC2 have not been fully elucidated. mTORC2 inhibits FOXO3A via S6K1 and AKT leading to increased longevity and regulates actin cytoskeleton assembly. Rictor and Sin1 are the two unique, essential mTORC2 components and Sin1 phosphorylation disassociates Sin1 from the complex suppressing mTORC2 kinase activity (Liu et al, 2013).

mTOR has been implicated in age-related pathologies and is considered a master regulator of cell growth and metabolism in response to nutrient cues. Rapamycin (Sirolimus) inhibits mTORC1 by binding to an abundant, intracellular protein, FKBP12 (FK506-binding protein) and disrupting the interaction between mTOR and raptor to decrease activity. Rapamycin does not directly inhibit mTORC2 but chronic exposure under some circumstances can lead to mTOR sequestration from mTORC2, inhibiting mTORC2 complex assembly. In addition to rapamycin, two mTOR compounds with the same mechanism of action are approved for clinical use, everolimus and temsirolimus for renal cell carcinoma and organ transplant rejection. Small molecule, dual inhibitors of mTORC1 and mTORC2 are in clinical development for a diverse range of oncology indications.

Idiopathic Pulmonary Fibrosis (IPF) is characterized by extracellular matrix (ECM) accumulation leading to structural distortion of lung architecture resulting in impaired gaseous exchange and death due to respiratory failure. Emerging evidence suggests cellular metabolic reprogramming may contribute to the pathogenesis of IPF including the observation of reproducibly increased ¹⁸fluorodeoxyglucose (FDG) pulmonary uptake in honeycombed lesion (Groves et al., 2009) elevated lung lactic acid levels promoting activation of the central profibrotic mediator, transforming growth factor (TGF)-β (Kottmann et al., 2012) and metabolic changes associated with fibroblast-to-myofibroblast transdifferentiation (Bernard et al., 2015). Energetic adaption maybe modulated by mTOR. In addition, inhibition of class I PI3k and mTOR has been shown to arrest fibroblast proliferation and collagen deposition in cells and tissue derived from patients with IPF (Mercer et al., 2015). mTOR is a critical effector of TGF-β in fibroblasts (Rahimi et al., 2009) and TGF-β has been implicated in diverse fibrotic conditions affecting the lung, kidney, skin and liver (for a review see Nanthakumar et al., 2015). More recently, TGF-β was shown to promote cardiac fibrosis (Khalil et al., 2017). Myofibroblasts are considered the primary pathogenic cell type during the development of a fibroproliferative response and subsequent organ fibrosis. Comparative studies using control and fibrotic myofibroblasts revealed aberrant translational regulation with dysregulated mTOR activity in disease-derived cells (Larsson et al., 2009).

Two drugs have been approved for the treatment of IPF, Esbriet (pirfenidone) and Ofev (nintedanib). Both slow down the progression of the disease but do not halt it and are are potentially associated with significant side effects and tolerability issues. The mechanism of action for Esbriet is not fully understood and patients are titrated with increasing doses but many patients fail to tolerate the recommended clinical dose

### SUMMARY OF THE INVENTION

The present invention provides a compound of formula (I)

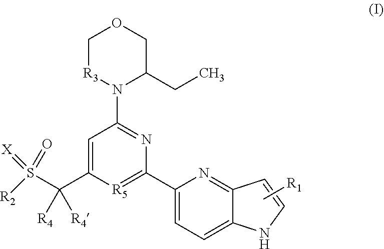

wherein:
X is O or NH;
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂;
R₃ is CH₂ or C═O;
R₄ and R_{4′} are both H, or R₄ and R_{4′} combine to form a 5- or 6-membered heterocycloalkylene which is unsubstituted or substituted with (C₁-C₃)alkyl; and
R₅ is CH or N;
wherein when R₅ is CH and R₁ is (C₂-C₃)alkyl, CH₂NH(C₁-C₃)alkyl, or CH₂OH, then R₃ is not CH₂;
or a pharmaceutically acceptable salt thereof.

The present invention also provides compounds of formulas (II), (IIa), (IIb), (IIc) and (III), as defined below, and pharmaceutically acceptable salts thereof.

The present invention provides a pharmaceutical composition comprising a compound according to each of formulas (I), (II), (IIa), (IIb), (IIc), or (III), or a pharmaceutically acceptable salt thereof, and a pharmaceutically acceptable carrier, diluent, or excipient.

The present invention relates to a method of treating a disease in which an mTOR kinase inhibitor is indicated, in a subject in need thereof, in particular a human subject in need thereof, comprising administering to said subject a therapeutically amount of a compound according to formulas (I), (II), (IIa), (IIb), (IIc), or (III), or pharmaceutically acceptable salt thereof.

The present invention also relates to a compound according to formulas (I), (II), (IIa), (IIb), (IIc), or (III), or a pharmaceutically acceptable salt thereof, for use in therapy.

In a further aspect, the invention relates to a compound according to formulas (I), (II), (IIa), (IIb), (IIc), or (III), or a pharmaceutically acceptable salt thereof, for use in the treatment of a disease in which an mTOR kinase inhibitor is indicated.

In a further aspect, the invention relates to a compound according to formula (I), (II), (IIa), (IIb), (IIc), or (III), or a pharmaceutically acceptable salt thereof, for use in the manufacture of a medicament for the treatment of a disease in which an mTOR kinase inhibitor is indicated.

In a further aspect, the invention relates to a pharmaceutical composition comprising a compound according to formulas (I), (II), (IIa), (IIb), (IIc), or (III), or a pharmaceutically acceptable salt thereof, and an additional therapeutic agent.

### DESCRIPTION OF FIGURES

FIG. 1 shows the XRPD spectrum of the crystal form of Example 35.

FIG. 2 shows the XRPD spectrum of the crystal form of Example 36.

### DETAILED DESCRIPTION OF THE INVENTION

The present invention provides a compound of formula (I)

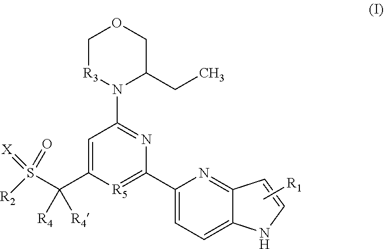

wherein:
X is O or NH;
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂;
R₃ is CH₂ or C═O;
R₄ and R_{4′} are both H, or R₄ and R₄′ combine to form a 5- or 6-membered heterocycloalkylene which is unsubstituted or substituted with (C₁-C₃)alkyl; and
R₅ is CH or N;
wherein when R₅ is CH and R₁ is (C₂-C₃)alkyl, CH₂NH(C₁-C₃)alkyl, or CH₂OH, then R₃ is not CH₂;
or a pharmaceutically acceptable salt thereof

In one embodiment, X is O.

For all formulas herein described which contain R₁, in one embodiment R₁ is methyl, ethyl, CH₂NHCH₃, CH₂NHCH₂CH₃, CH₂—OH, or CH₂CH₂—OH. In one aspect, R₁ is methyl, ethyl, CH₂NHCH₃, or CH₂OH. In yet another aspect, R₁ is methyl, CH₂NHCH₃, or CH₂OH.

For all formulas herein described which contain R₂, in one embodiment R₂ is methyl, ethyl, N(H)CH₃, N(H)CH₂CH₃, N(CH₃)₂, N(CH₂CH₃)₂, or NH₂. In one aspect, R₂ is methyl, n-propyl, isopropyl, N(H)CH₃, N(CH₃)₂, or NH₂. In one aspect, R₂ is methyl, N(H)CH₃, N(CH₃)₂, or NH₂.

For all formulas herein described which contain R₃, in one embodiment R₃ is CH₂.

For all formulas herein described which contain R₃, in an alternative embodiment R₃ is C═O.

For all formulas herein described which contain R₄ and R₄′, in one embodiment, R₄ and R₄′ are both H.

For all formulas herein described which contain R₄ and R₄′, in an alternative embodiment, R₄ and R₄′ combine to form a 5- or 6-membered heterocycloalkylene which is unsubstituted or substituted with methyl or ethyl. In one aspect, the 5- or 6-membered heterocycloalkylene contains at least one heteroatom which is O, N, or S. In one aspect, the 5- or 6-membered heterocycloalkylene contains at least one heteroatom which is O or N. In another aspect, the 5- or 6-membered heterocycloalkylene contains one the heteroatom which is O or N.

For all formulas herein described which contain R₅, in one embodiment R₅ is CH.

For all formulas herein described which contain R₅, in an alternative embodiment R₅ is N.

In one embodiment, the invention relates to a compound or pharmaceutically acceptable salt thereof, of formula (II)

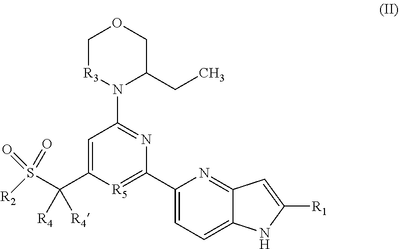

wherein:
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂;
R₃ is CH₂ or C═O;
R₄ and R₄′ are both H, or R₄ and R₄′ combine to form a 5- or 6-membered heterocycloalkylene which is unsubstituted or substituted with (C₁-C₃)alkyl; and
R₅ is CH or N;
wherein when R₅ is CH and R₁ is (C₂-C₃)alkyl, CH₂NH(C₁-C₃)alkyl, or CH₂OH, then R₃ is not CH₂.

In one embodiment, for the compound of Formula (II) or (II′);
R₁ is methyl, ethyl, CH₂NHCH₃, or CH₂OH;
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with methyl;
or a pharmaceutically acceptable salt thereof.

In one embodiment, the invention relates to a compound thereof of formula (IIa)

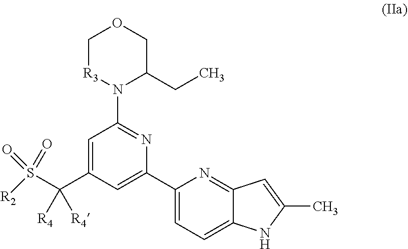

wherein:
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof;
or the compound is of formula (IIb)

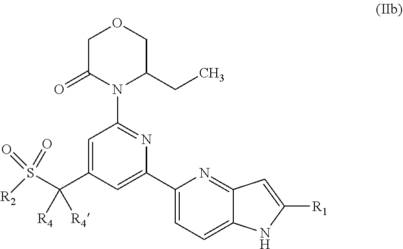

wherein:
R₁ is (C₁-C₃)alkyl, CH₂NH(C₁-C₃)alkyl, or (C₁-C₃)alkyl-OH;
R₂ is methyl, N(H)CH₃, N(CH₃)₂, or NH₂; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof;
or the compound is of formula (IIc)

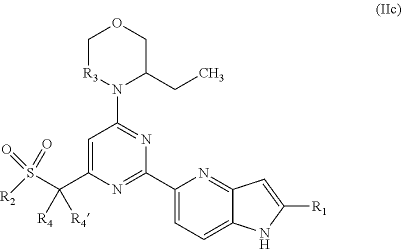

wherein:
R₁ is (C₁-C₃)alkyl, CH₂NH(C₁-C₃)alkyl, or (C₁-C₃)alkyl-OH;
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof.

In one embodiment, the invention relates to a compound, of formula (IIa)

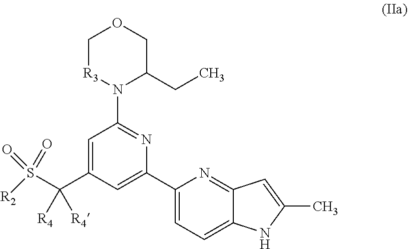

wherein:
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof

In one embodiment, the invention relates to a compound of formula (IIb)

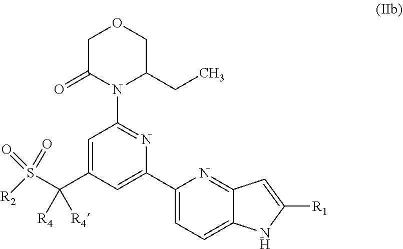

wherein:
R₁ is (C₁-C₃)alkyl, CH₂NH(C₁-C₃)alkyl, or (C₁-C₃)alkyl-OH;
R₂ is methyl, N(H)CH₃, N(CH₃)₂, or NH₂; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof.

In one embodiment, the invention relates to a compound of formula (IIc)

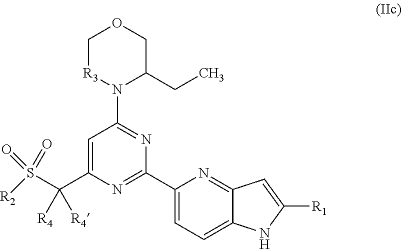

wherein:
R₁ is (C₁-C₃)alkyl, CH₂NH(C₁-C₃)alkyl, or (C₁-C₃)alkyl-OH;
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof.

In one embodiment, the invention relates to a compound of formula (III)

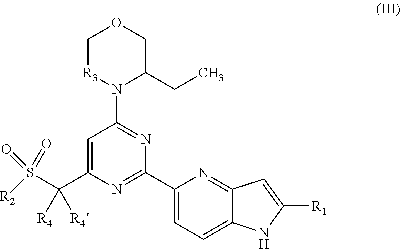

wherein:
R₁ is CH₂NH(C₁-C₃)alkyl;
R₂ is isopropyl, or NH₂;
R₃ is CH₂; and
R₄ and R₄′ are both H;
or a pharmaceutically acceptable salt thereof.

In one embodiment, there is provided a compound, which is:
[(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine;
(S)-(5-(4-(3-ethylmorpholino)-6-((methylsulfonyl)methyl)pyrimidin-2-yl)-1H-pyrrolo[3,2-b]pyridin-2-yl)methanol;
[(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl](methyl)amine;
(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methanol;
(5S)-5-ethyl-4-{2-[2-(hydroxymethyl)-1H-pyrrolo[3,2-b]pyridin-5-yl]-6-(methanesulfonylmethyl)pyrimidin-4-yl}morpholin-3-one;
{6-[(3S)-3-ethylmorpholin-4-yl]-2-{2-[(methylamino)methyl]-1H-pyrrolo[3,2-b]pyridin-5-yl}pyrimidin-4-yl}methanesulfonamide;
(S)-1-(2-(3-ethyl-5-oxomorpholino)-6-(2-((methylamino)methyl)-1H-pyrrolo[3,2-b]pyridin-5-yl)pyridin-4-yl)-N,N-dimethylmethanesulfonamide;
1-(5-(6-((2R,3R)-2,3-dimethylmorpholino)-4-((methylsulfonyl)methyl)pyridin-2-yl)-1Hpyrrolo[3,2-b]pyridin-2-yl)-Nmethylmethanamine;
1-{6-[(3S)-3-ethylmorpholin-4-yl]-2-[2-(hydroxymethyl)-1H-pyrrolo[3,2-b]pyridin-5-yl]pyrimidin-4-yl}-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N-methylmethanesulfonamide;
1-{6-[(3S)-3-ethylmorpholin-4-yl]-2-{2-methyl-1H-pyrrolo[3,2-b]pyridin-5-yl}pyrimidin-4-yl}-N-methylmethanesulfonamide;
(3S)-3-ethyl-4-[6-(methanesulfonylmethyl)-2-{2-methyl-1H-pyrrolo[3,2-b]pyridin-5-yl}pyrimidin-4-yl]morpholine;
(3S)-3-ethyl-4-[6-(4-methanesulfonyloxan-4-yl)-2-{2-methyl-1H-pyrrolo[3,2-b]pyridin-5-yl}pyrimidin-4-yl]morpholine;
(5S)-5-ethyl-4-[6-(methanesulfonylmethyl)-2-{2-methyl-1H-pyrrolo[3,2-b]pyridin-5-yl}pyrimidin-4-yl]morpholin-3-one;
(5S)-5-ethyl-4-(2-{2-ethyl-1H-pyrrolo[3,2-b]pyridin-5-yl}-6-(methanesulfonylmethyl)pyrimidin-4-yl)morpholin-3-one;
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(3S)-3-ethyl-4-[6-(4-methanesulfonylpiperidin-4-yl)-2-{2-methyl-1H-pyrrolo[3,2-b]pyridin-5-yl}pyrimidin-4-yl]morpholine;
(S)-3-ethyl-4-(6-(2-methyl-1H-pyrrolo[3,2-b]pyridin-5-yl)-4-(4-(methylsulfonyl)piperidin-4-yl)pyridin-2-yl)morpholine;
(S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine;
(S)-(5-(6-(3-ethylmorpholino)-4-(1-methyl-4-(methylsulfonyl)piperidin-4-yl)pyridin-2-yl)-1H-pyrrolo[3,2-b]pyridin-2-yl)methanol;
(S)-3-ethyl-4-(6-(2-methyl-1H-pyrrolo[3,2-b]pyridin-5-yl)-4-(4-(methylsulfonyl)tetra-hydro-2H-pyran-4-yl)pyridin-2-yl)morpholine;
(S)-3-ethyl-4-(6-(2-methyl-1H-pyrrolo[3,2-b]pyridin-5-yl)-4-((methylsulfonyl)methyl) pyridin-2-yl)morpholine;
(S)-(2-(3-ethylmorpholino)-6-(2-methyl-1H-pyrrolo[3,2-b]pyridin-5-yl)pyridin-4-yl)methanesulfonamide;
(S)-5-ethyl-4-(6-(2-methyl-1H-pyrrolo[3,2-b]pyridin-5-yl)-4-((methylsulfonyl) methyl)pyridin-2-yl)morpholin-3-one;
(S)-5-ethyl-4-(6-(2-methyl-1H-pyrrolo[3,2-b]pyridin-5-yl)-4-(4-(methylsulfonyl) tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-(4-(methylsulfonyl)tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-((methylsulfonyl)
ethyl)pyridin-2-yl)morpholin-3-one;
(S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one;
(S)-1-(2-(3-ethyl-5-oxomorpholino)-6-(2-(hydroxymethyl)-1H-pyrrolo[3,2-b]pyridin-5-yl)pyridin-4-yl)-N,Ndimethylmethanesulfonamide;
(S)—N,N-diethyl-1-(6-(3-ethylmorpholino)-2-(2-((methylamino)methyl)-1H-pyrrolo[3,2-b]pyridin-5-yl)pyrimidin-4-yl)methanesulfonamide;
(S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((propylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine; or

or a pharmaceutically acceptable salt thereof.

In one embodiment, there is provided a compound, which is:
[(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine;
(S)-(5-(4-(3-ethylmorpholino)-6-((methylsulfonyl)methyl)pyrimidin-2-yl)-1H-pyrrolo[3,2-b]pyridin-2-yl)methanol;
[(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl](methyl)amine;
(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methanol;
(5S)-5-ethyl-4-{2-[2-(hydroxymethyl)-1H-pyrrolo[3,2-b]pyridin-5-yl]-6-(methanesulfonylmethyl)pyrimidin-4-yl}morpholin-3-one;
{6-[(3S)-3-ethylmorpholin-4-yl]-2-{2-[(methylamino)methyl]-1H-pyrrolo[3,2-b]pyridin-5-yl}pyrimidin-4-yl}methanesulfonamide;
(S)-1-(2-(3-ethyl-5-oxomorpholino)-6-(2-((methylamino)methyl)-1H-pyrrolo[3,2-b]pyridin-5-yl)pyridin-4-yl)-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N-methylmethanesulfonamide;
1-{6-[(3S)-3-ethylmorpholin-4-yl]-2-{2-methyl-1H-pyrrolo[3,2-b]pyridin-5-yl}pyrimidin-4-yl}-N-methylmethanesulfonamide;
(3S)-3-ethyl-4-[6-(methanesulfonylmethyl)-2-{2-methyl-1H-pyrrolo[3,2-b]pyridin-5-yl}pyrimidin-4-yl]morpholine;
(3S)-3-ethyl-4-[6-(4-methanesulfonyloxan-4-yl)-2-{2-methyl-1H-pyrrolo[3,2-b]pyridin-5-yl}pyrimidin-4-yl]morpholine;
(5S)-5-ethyl-4-[6-(methanesulfonylmethyl)-2-{2-methyl-1H-pyrrolo[3,2-b]pyridin-5-yl}pyrimidin-4-yl]morpholin-3-one;
(5S)-5-ethyl-4-(2-{2-ethyl-1H-pyrrolo[3,2-b]pyridin-5-yl}-6-(methanesulfonylmethyl)pyrimidin-4-yl)morpholin-3-one;
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(3S)-3-ethyl-4-[6-(4-methanesulfonylpiperidin-4-yl)-2-{2-methyl-1H-pyrrolo[3,2-b]pyridin-5-yl}pyrimidin-4-yl]morpholine;
(S)-3-ethyl-4-(6-(2-methyl-1H-pyrrolo[3,2-b]pyridin-5-yl)-4-(4-(methylsulfonyl)piperidin-4-yl)pyridin-2-yl)morpholine;
(S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine;
(S)-(5-(6-(3-ethylmorpholino)-4-(1-methyl-4-(methylsulfonyl)piperidin-4-yl)pyridin-2-yl)-1H-pyrrolo[3,2-b]pyridin-2-yl)methanol;
(S)-3-ethyl-4-(6-(2-methyl-1H-pyrrolo[3,2-b]pyridin-5-yl)-4-(4-(methylsulfonyl)tetra-hydro-2H-pyran-4-yl)pyridin-2-yl)morpholine;
(S)-3-ethyl-4-(6-(2-methyl-1H-pyrrolo[3,2-b]pyridin-5-yl)-4-((methylsulfonyl)methyl) pyridin-2-yl)morpholine;
(S)-(2-(3-ethylmorpholino)-6-(2-methyl-1H-pyrrolo[3,2-b]pyridin-5-yl)pyridin-4-yl)methanesulfonamide;
(S)-5-ethyl-4-(6-(2-methyl-1H-pyrrolo[3,2-b]pyridin-5-yl)-4-((methylsulfonyl) methyl)pyridin-2-yl)morpholin-3-one;
(S)-5-ethyl-4-(6-(2-methyl-1H-pyrrolo[3,2-b]pyridin-5-yl)-4-(4-(methylsulfonyl) tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-(4-(methylsulfonyl)tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-((methylsulfonyl)
ethyl)pyridin-2-yl)morpholin-3-one;
(S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one;
(S)-1-(2-(3-ethyl-5-oxomorpholino)-6-(2-(hydroxymethyl)-1H-pyrrolo[3,2-b]pyridin-5-yl)pyridin-4-yl)-N,Ndimethylmethanesulfonamide;
(S)—N,N-diethyl-1-(6-(3-ethylmorpholino)-2-(2-((methylamino)methyl)-1H-pyrrolo[3,2-b]pyridin-5-yl)pyrimidin-4-yl)methanesulfonamide;
(S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((propylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine; or

or a pharmaceutically acceptable salt thereof.

In one embodiment, there is provided a compound, which is:
[(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine;
(S)-(5-(4-(3-ethylmorpholino)-6-((methylsulfonyl)methyl)pyrimidin-2-yl)-1H-pyrrolo[3,2-b]pyridin-2-yl)methanol;
[(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl](methyl)amine;
(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methanol;
(5S)-5-ethyl-4-{2-[2-(hydroxymethyl)-1H-pyrrolo[3,2-b]pyridin-5-yl]-6-(methanesulfonylmethyl)pyrimidin-4-yl}morpholin-3-one;
{6-[(3S)-3-ethylmorpholin-4-yl]-2-{2-[(methylamino)methyl]-1H-pyrrolo[3,2-b]pyridin-5-yl}pyrimidin-4-yl}methanesulfonamide;
(S)-1-(2-(3-ethyl-5-oxomorpholino)-6-(2-((methylamino)methyl)-1H-pyrrolo[3,2-b]pyridin-5-yl)pyridin-4-yl)-N,N-dimethylmethanesulfonamide;
1-(5-(6-((2R,3R)-2,3-dimethylmorpholino)-4-((methylsulfonyl)methyl)pyridin-2-yl)-1Hpyrrolo[3,2-b]pyridin-2-yl)-Nmethylmethanamine;
1-{6-[(3S)-3-ethylmorpholin-4-yl]-2-[2-(hydroxymethyl)-1H-pyrrolo[3,2-b]pyridin-5-yl]pyrimidin-4-yl}-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N-methylmethanesulfonamide;
1-{6-[(3S)-3-ethylmorpholin-4-yl]-2-{2-methyl-1H-pyrrolo[3,2-b]pyridin-5-yl}pyrimidin-4-yl}-N-methylmethanesulfonamide;
(3S)-3-ethyl-4-[6-(methanesulfonylmethyl)-2-{2-methyl-1H-pyrrolo[3,2-b]pyridin-5-yl}pyrimidin-4-yl]morpholine;
(3S)-3-ethyl-4-[6-(4-methanesulfonyloxan-4-yl)-2-{2-methyl-1H-pyrrolo[3,2-b]pyridin-5-yl}pyrimidin-4-yl]morpholine;
(5S)-5-ethyl-4-[6-(methanesulfonylmethyl)-2-{2-methyl-1H-pyrrolo[3,2-b]pyridin-5-yl}pyrimidin-4-yl]morpholin-3-one;
(5S)-5-ethyl-4-(2-{2-ethyl-1H-pyrrolo[3,2-b]pyridin-5-yl}-6-(methanesulfonylmethyl)pyrimidin-4-yl)morpholin-3-one;
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(3S)-3-ethyl-4-[6-(4-methanesulfonylpiperidin-4-yl)-2-{2-methyl-1H-pyrrolo[3,2-b]pyridin-5-yl}pyrimidin-4-yl]morpholine;
(S)-3-ethyl-4-(6-(2-methyl-1H-pyrrolo[3,2-b]pyridin-5-yl)-4-(4-(methylsulfonyl)piperidin-4-yl)pyridin-2-yl)morpholine;
(S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine;
(S)-(5-(6-(3-ethylmorpholino)-4-(1-methyl-4-(methylsulfonyl)piperidin-4-yl)pyridin-2-yl)-1H-pyrrolo[3,2-b]pyridin-2-yl)methanol;
(S)-3-ethyl-4-(6-(2-methyl-1H-pyrrolo[3,2-b]pyridin-5-yl)-4-(4-(methylsulfonyl)tetra-hydro-2H-pyran-4-yl)pyridin-2-yl)morpholine;
(S)-3-ethyl-4-(6-(2-methyl-1H-pyrrolo[3,2-b]pyridin-5-yl)-4-((methylsulfonyl)methyl) pyridin-2-yl)morpholine;
(S)-(2-(3-ethylmorpholino)-6-(2-methyl-1H-pyrrolo[3,2-b]pyridin-5-yl)pyridin-4-yl)methanesulfonamide;
(S)-5-ethyl-4-(6-(2-methyl-1H-pyrrolo[3,2-b]pyridin-5-yl)-4-((methylsulfonyl) methyl)pyridin-2-yl)morpholin-3-one;
(S)-5-ethyl-4-(6-(2-methyl-1H-pyrrolo[3,2-b]pyridin-5-yl)-4-(4-(methylsulfonyl) tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-(4-(methylsulfonyl)tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-((methylsulfonyl)
ethyl)pyridin-2-yl)morpholin-3-one;
(S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one; or
(S)-1-(2-(3-ethyl-5-oxomorpholino)-6-(2-(hydroxymethyl)-1H-pyrrolo[3,2-b]pyridin-5-yl)pyridin-4-yl)-N,Ndimethylmethanesulfonamide;

or a pharmaceutically acceptable salt thereof.

In one embodiment, there is provided a compound, which is:
[(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine;
(S)-(5-(4-(3-ethylmorpholino)-6-((methylsulfonyl)methyl)pyrimidin-2-yl)-1H-pyrrolo[3,2-b]pyridin-2-yl)methanol;
[(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl](methyl)amine;
(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methanol;
(5S)-5-ethyl-4-{2-[2-(hydroxymethyl)-1H-pyrrolo[3,2-b]pyridin-5-yl]-6-(methanesulfonylmethyl)pyrimidin-4-yl}morpholin-3-one;
{6-[(3S)-3-ethylmorpholin-4-yl]-2-{2-[(methylamino)methyl]-1H-pyrrolo[3,2-b]pyridin-5-yl}pyrimidin-4-yl}methanesulfonamide;
(S)-1-(2-(3-ethyl-5-oxomorpholino)-6-(2-((methylamino)methyl)-1H-pyrrolo[3,2-b]pyridin-5-yl)pyridin-4-yl)-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N-methylmethanesulfonamide;
1-{6-[(3S)-3-ethylmorpholin-4-yl]-2-{2-methyl-1H-pyrrolo[3,2-b]pyridin-5-yl}pyrimidin-4-yl}-N-methylmethanesulfonamide;
(3S)-3-ethyl-4-[6-(methanesulfonylmethyl)-2-{2-methyl-1H-pyrrolo[3,2-b]pyridin-5-yl}pyrimidin-4-yl]morpholine;
(3S)-3-ethyl-4-[6-(4-methanesulfonyloxan-4-yl)-2-{2-methyl-1H-pyrrolo[3,2-b]pyridin-5-yl}pyrimidin-4-yl]morpholine;
(5S)-5-ethyl-4-[6-(methanesulfonylmethyl)-2-{2-methyl-1H-pyrrolo[3,2-b]pyridin-5-yl}pyrimidin-4-yl]morpholin-3-one;
(5S)-5-ethyl-4-(2-{2-ethyl-1H-pyrrolo[3,2-b]pyridin-5-yl}-6-(methanesulfonylmethyl)pyrimidin-4-yl)morpholin-3-one;
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(3S)-3-ethyl-4-[6-(4-methanesulfonylpiperidin-4-yl)-2-{2-methyl-1H-pyrrolo[3,2-b]pyridin-5-yl}pyrimidin-4-yl]morpholine;
(S)-3-ethyl-4-(6-(2-methyl-1H-pyrrolo[3,2-b]pyridin-5-yl)-4-(4-(methylsulfonyl)piperidin-4-yl)pyridin-2-yl)morpholine;
(S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine;
(S)-(5-(6-(3-ethylmorpholino)-4-(1-methyl-4-(methylsulfonyl)piperidin-4-yl)pyridin-2-yl)-1H-pyrrolo[3,2-b]pyridin-2-yl)methanol;
(S)-3-ethyl-4-(6-(2-methyl-1H-pyrrolo[3,2-b]pyridin-5-yl)-4-(4-(methylsulfonyl)tetra-hydro-2H-pyran-4-yl)pyridin-2-yl)morpholine;
(S)-3-ethyl-4-(6-(2-methyl-1H-pyrrolo[3,2-b]pyridin-5-yl)-4-((methylsulfonyl)methyl) pyridin-2-yl)morpholine;
(S)-(2-(3-ethylmorpholino)-6-(2-methyl-1H-pyrrolo[3,2-b]pyridin-5-yl)pyridin-4-yl)methanesulfonamide;
(S)-5-ethyl-4-(6-(2-methyl-1H-pyrrolo[3,2-b]pyridin-5-yl)-4-((methylsulfonyl) methyl)pyridin-2-yl)morpholin-3-one;
(S)-5-ethyl-4-(6-(2-methyl-1H-pyrrolo[3,2-b]pyridin-5-yl)-4-(4-(methylsulfonyl) tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-(4-(methylsulfonyl)tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-((methylsulfonyl)
ethyl)pyridin-2-yl)morpholin-3-one;
(S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one; or
(S)-1-(2-(3-ethyl-5-oxomorpholino)-6-(2-(hydroxymethyl)-1H-pyrrolo[3,2-b]pyridin-5-yl)pyridin-4-yl)-N,Ndimethylmethanesulfonamide;

or a pharmaceutically acceptable salt thereof.

In one embodiment, there is provided a compound which is:
(S)—N,N-diethyl-1-(6-(3-ethylmorpholino)-2-(2-((methylamino)methyl)-1H-pyrrolo[3,2-b]pyridin-5-yl)pyrimidin-4-yl)methanesulfonamide;
(S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((propylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine;

or a pharmaceutically acceptable salt thereof.

In one embodiment, the compound is:
[(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine;
(5S)-5-ethyl-4-[6-(methanesulfonylmethyl)-2-{2-methyl-1H-pyrrolo[3,2-b]pyridin-5-yl}pyrimidin-4-yl]morpholin-3-one;
(5S)-5-ethyl-4-(2-{2-ethyl-1H-pyrrolo[3,2-b]pyridin-5-yl}-6-(methanesulfonylmethyl)pyrimidin-4-yl)morpholin-3-one;
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(S)-3-ethyl-4-(6-(2-methyl-1H-pyrrolo[3,2-b]pyridin-5-yl)-4-(4-(methylsulfonyl)piperidin-4-yl)pyridin-2-yl)morpholine; or
(S)-5-ethyl-4-(6-(2-methyl-1H-pyrrolo[3,2-b]pyridin-5-yl)-4-((methylsulfonyl) methyl)pyridin-2-yl)morpholin-3-one;

or a pharmaceutically acceptable salt thereof.

In one embodiment, the compound is 1-(5-(6-((2R,3R)-2,3-dimethylmorpholino)-4-((methylsulfonyl)methyl)pyridin-2-yl)-1Hpyrrolo[3,2-b]pyridin-2-yl)-Nmethylmethanamine; or a pharmaceutically acceptable salt thereof.
In one embodiment, the compound is selected from:
1-{6-[3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
1-(5-(4-(3-ethylmorpholino)-6-((ethylsulfonyl)methyl)pyrimidin-2-yl)-1H-pyrrolo[3,2-b]pyridin-2-yl)-N-methylmethanamine

or a pharmaceutically acceptable salt thereof.

In one embodiment, the compound is selected from:
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine

or a pharmaceutically acceptable salt thereof.

In one embodiment, the compound is:
1-{6-[3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;

or a pharmaceutically acceptable salt thereof.

In one embodiment, the compound is:
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;

or a pharmaceutically acceptable salt thereof.

In one embodiment, the compound is:
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide benzoate.

In one embodiment, the compound is:
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide.

As used herein, a compound of the invention is a compound of any one of formulas (I), (II), (IIa), (IIb), (IIc) or (III).

The compounds of the invention may contain one or more chiral centres, so that optical isomers, e.g. diastereoisomers may be formed. Accordingly, the present invention encompasses such isomers of the compounds of the invention whether as individual isomers isolated such as to be substantially free of the other isomer (i.e. pure) or as mixtures. An individual isomer isolated such as to be substantially free of the other isomer (i.e. pure) may be isolated such that less than 10%, particularly less than about 1%, for example less than about 0.1% of the other isomer is present.

Separation of isomers may be achieved by conventional techniques known to those skilled in the art, e.g. by fractional crystallisation, chromatography, HPLC or a combination of these techniques.

In one embodiment, the ethyl substituent on the morpholine ring is in the following stereochemical configuration (the S-diastereoisomer):

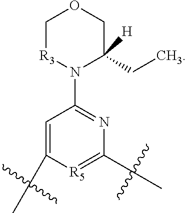

In another embodiment, the ethyl substituent on the morpholine ring is in the following stereochemical configuration (the R-diastereoisomer);

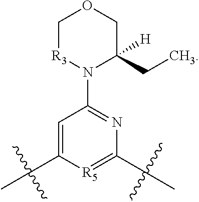

It is to be understood that the references herein to a compound of the invention or a pharmaceutically acceptable salt thereof includes a compound of the invention as a free base, or as a pharmaceutically acceptable salt thereof. Thus, in one embodiment, the invention is directed to a compound of formulas (I), (II), (IIa), (IIb), (IIc), and (III). In another embodiment, the invention is directed to a pharmaceutically acceptable salt of a compound of formulas (I), (II), (IIa), (IIb), (IIc) and (III). In another embodiment, the invention is directed to a pharmaceutically acceptable salt of a compound of formulas (I), (II), (IIa), (IIb), (IIc) and (III).

Non-pharmaceutically acceptable salts are within the scope of the present invention, for example for use as intermediates in the preparation of a compound of the invention or a pharmaceutically acceptable salt thereof.

Suitable pharmaceutically acceptable salts can include acid addition salts.

Such acid addition salts can be formed by reaction of a compound of the invention (which, for example contains a basic amine or other basic functional group) with the appropriate acid, optionally in a suitable solvent such as an organic solvent, to give the salt which can be isolated by a variety of methods, including crystallisation and filtration.

Salts may be prepared in situ during the final isolation and purification of a compound of the invention. If a basic compound of the invention is isolated as a salt, the corresponding free base form of that compound may be prepared by any suitable method known to the art, including treatment of the salt with an inorganic or organic base.

It will be understood that if a compound of the invention contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of the invention are included within the scope of the invention, including sub-stoichiometric salts, for example where a counterion contains more than one acidic proton.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

The compounds of the invention may exist in a crystalline or non crystalline form, or as a mixture thereof. Pharmaceutically acceptable solvates may be formed for crystalline or non-crystalline compounds. In crystalline solvates, solvent molecules are incorporated into the crystalline lattice during crystallisation. Solvates may include non-aqueous solvents such as, but not limited to, ethanol, isopropanol, DMSO, acetic acid, ethanolamine, ethyl acetate, MeOH/TBME, MeCN/TBME or MeCN/Heptane or that may involve water as the solvent that is incorporated into the crystalline lattice. Solvates wherein water is the solvent incorporated into the crystalline lattice are typically referred to as “hydrates”. Hydrates include stoichiometric hydrates as well as compositions containing variable amounts of water.

Compounds of the invention that exist in crystalline form, including the various solvates thereof, may exhibit polymorphism (i.e. the capacity to occur in different crystalline structures). These different crystalline forms are typically known as “polymorphs”. Polymorphs have the same chemical composition, but differ in packing, geometrical arrangement, and other descriptive properties of the crystalline solid state. Polymorphs, therefore, may have different physical properties such as shape, density, hardness, deformability, stability and dissolution properties. Polymorphs typically exhibit different melting points, IR spectra, and X-ray diffraction patterns, which may be used for identification. Different polymorphs may be produced, for example, by changing or adjusting the reaction conditions or reagents used in making the compound. For example, changes in temperature, pressure, or solvent may result in polymorphs. In addition, one polymorph may spontaneously convert to another polymorph under certain conditions.

In one embodiment, the compound of the invention is 1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide, and the crystalline form is Crystal Form A, as described in Example 35.

In one embodiment, the compound of the invention 1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide, and the crystalline form is Crystal From B, as described in Example 36.

### Definitions

As used herein, the term “alkyl” represents a saturated, straight or branched hydrocarbon moiety having the specified number of carbon atoms. The term “(C₁-C₃)alkyl” refers to an unsubstituted alkyl moiety containing 1, 2 or 3 carbon atoms; exemplary alkyls include methyl, ethyl and propyl.

As used herein, the term “(C₁-C₃)alkyl-OH” refers to a straight chain (C₁-C₃)alkyl group with a hydroxyl group at the C-1, C-2, or C-3 positions accordingly.

As used herein, the term “5- or 6-membered heterocycloalkylene” refers to a 5- or 6-membered cyclic moiety containing 4 or 5 carbon atoms in addition to 1 or 2 oxygen, sulphur or nitrogen atoms, with two points of attachment from the same or different carbon atoms. In one embodiment, the heterocycloalkylene group contains 1 oxygen and 1 nitrogen atom. In one embodiment, the heterocycloalkylene group contains 1 oxygen and 1 nitrogen atom. In one embodiment, the heterocycloalkylene group contains 1 oxygen atom. In one embodiment, the heterocycloalkylene group contains 1 nitrogen atom.

As used herein, the term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function.

“Pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio. Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see http://www.wiley.com/WileyCDA/WileyTitle/productCd-3906390519.html).

### Statement of Use

The compounds of the invention and pharmaceutically acceptable salts thereof are believed to be inhibitors of mTOR kinase, and thus have potential utility in the treatment of diseases or conditions for which an mTOR kinase inhibitor is indicated.

Thus, in one aspect of the invention, there is provided a compound of formulas (I), (II), (IIa), (IIb), (IIc), and (III) or a pharmaceutically acceptable salt thereof for use in therapy. The compound of formulas (I), (II), (IIa), (IIb), (IIc), and (III) or a pharmaceutically acceptable salt thereof can be for use in the treatment of a disease or condition for which an mTOR kinase inhibitor is indicated.

In one aspect of the invention, there is provided a compound of formulas (I), (II), (IIa), (IIb), and (IIc), or a pharmaceutically acceptable salt thereof for the manufacture of a medicament for the treatment of a disease or condition for which an mTOR kinase inhibitor is indicated.

In one aspect of the invention, there is provided a method of treating a disease in which an mTOR kinase inhibitor is indicated in a subject in need thereof comprising administering to said subject a therapeutically amount of a compound according to formulas (I), (II), (IIa), (IIb), (IIc), and (III) or a pharmaceutically acceptable salt thereof. In one embodiment of the invention, the subject in need thereof is a human subject.

Fibrotic diseases involve the formation of excess fibrous connective tissue in an organ or tissue in a reparative or reactive process. Inhibitors of mTOR are believed to be useful in the treatment of a variety of such diseases or conditions including those dependent on mTOR function. Diseases may include, but are not limited to lung fibrosis e.g. Idiopathic pulmonary fibrosis (IPF), Non-specific interstitial pneumonia (NSIP), Hypersensitivity pneumonitis (HP), Usual interstitial pneumonitis (UIP), Interstitial lung disease (ILD), progressive massive fibrosis, coal workers' pneumoconiosis, pigeon fancier's lung, familial pulmonary fibrosis, pulmonary fibrosis, connective tissue-interstitial lung disease (RA-ILD, SSc-ILD), Hermansky-Pudlak syndrome, airway fibrosis in asthma, airway fibrosis in COPD, ARDS associated fibrosis, acute lung injury, radiation-induced fibrosis, drug-induced fibrosis and pulmonary hypertension. Other pulmonary indications in which inhibitors of mTOR may be useful include COPD, lymphangioleiomyomatosis (LAM), obliterative bronchiolitis, asthma and granulomatous diseases such as sarcoidosis.

Non-lung fibrosis conditions in which inhibitors of mTOR may be useful include renal fibrosis (chronic kidney disease (CKD), end-stage renal disease (ESRD), diabetic nephropathy, IgA nephropathy, lupus nephritis, focal segmental glomerulosclerosis (FSGS), tubulointerstitial fibrosis, transplant nephropathy, autoimmune nephropathy, drug-induced nephropathy, hypertension-related nephropathy, nephrogenic systemic fibrosis); hepatic fibrosis (virally-induced fibrosis (e.g. hepatitis C or B), autoimmune hepatitis, primary biliary cirrhosis, alcoholic liver disease, non-alcoholic fatty liver disease (NAFLD) including non-alcoholic steatohepatitis (NAS H), congenital hepatic fibrosis, primary sclerosing cholangitis, drug-induced hepatitis, hepatic cirrhosis); skin fibrosis (hypertrophic scars, scleroderma, keloid scarring, dermatomyositis, eosinophilic fasciitis, Dupytrens contracture, Ehlers-Danlos syndrome, Peyronie's disease, epidermolysis bullosa dystrophica, oral submucous fibrosis); ocular fibrosis (age-related macular degeneration (AMD), diabetic macular oedema, dry eye, glaucoma) corneal scarring, corneal injury and corneal wound healing, prevention of filter bleb scarring post trabeculectomy surgery; cardiac fibrosis (congestive heart failure, atherosclerosis, myocardial infarction, endomyocardial fibrosis, hypertrophic cardiomyopathy (HCM)) and other miscellaneous fibrotic conditions (mediastinal fibrosis, myelofibrosis, retroperitoneal fibrosis, Crohn's disease, neurofibromatosis, uterine leiomyomas (fibroids), chronic organ transplant rejection.

In addition, oncology indications in which inhibitors of mTOR may be useful include Pre-cancerous lesions or cancers associated with mTOR (endometrial, basal cell, liver, colon, cervical, oral, pancreas, breast and ovarian cancers, Kaposi's sarcoma, giant cell tumours and cancer associated stroma); non-small cell lung cancer; non-Hodgkin's lymphoma, relapsed or refractory advanced solid tumours, advanced malignant solid neoplasm, locally advanced or metastatic solid tumours and soft tissue sarcomas.

Furthermore, diseases characterized by mutations in PI3k/mTOR including tuberous sclerosis, Smith-Kingsmore syndrome, focal cortical dysplasia and oncology indications, those conditions where the treatment of rapalogues such as sirolimus and everolimus are permitted (transplant recipients, rescue immunosuppression and chronic graft versus host disease) and diseases related to obesity (adipose tissue inflammation) and metabolic disorders (diabetes) or diseases related to ageing.

The term “disease or condition for which an mTOR kinase inhibitor is indicated” is intended to include any or all of the above disease states.

In one embodiment the disease or condition for which an mTOR kinase inhibitor is indicated is pulmonary fibrosis including idiopathic pulmonary fibrosis and any condition characterised by excessive tissue scarring affecting the skin, liver, kidney or heart. In a further embodiment the disease or condition for which an mTOR kinase inhibitor is indicated is idiopathic pulmonary fibrosis.

### Biomarkers

Clinically, mTOR activity may be assessed in combination with recently identified biomarkers shown to correlate with disease severity (BGM, C1M, C3A, C3M, C6M, CRPM) in a cohort of patients with IPF or NSIP (Jenkins et al., 2015).
In one embodiment, there is provided a method for treating a subject suffering from idiopathic pulmonary fibrosis, the method comprising:
a) detecting an amount of one, two, three, four, five or six biomarkers selected from the group consisting of BGM, C1M, C3A, C3M, C6M, or CRPM in a sample of the subject;
b) comparing the amount of the one, two, three, four, five or six biomarkers to a reference amount of the one, two, three, four, five or six biomarkers;
c) identifying the subject as having an increased risk for disease progression if the amount of the one, two, three, four, five or six biomarkers in the sample is greater than the reference amount of the one, two, three, four, five or six biomarkers; and
d) treating the subject with a compound of the invention or a pharmaceutical acceptable salt thereof if the subject is identified as having an increased risk for disease progression.

Additional biomarkers of collagen synthesis (PRO-C3, PRO-C6, P1NP) may also be used to measure a therapeutic response to mTOR modulation in patients. Serum levels of PRO-C3 and PRO-C6 correlate with disease progression in patients with IPF (conference poster ICLAF 2018).

Therefore, in one embodiment, there is provided an in vitro method for monitoring treatment of a subject diagnosed with idiopathic pulmonary fibrosis, comprising:
a) Determining the amount of one, two or three biomarkers, selected from the group consisting of PRO-C3, PRO-C6, and P1NP in a first baseline biological sample of a patient
b) Treating the subject with a compound of the invention
c) Determining the amount of one, two or three biomarkers, selected from the group consisting of PRO-C3, PRO-C6, and P1NP in a second biological sample of a patient, taken on a separate occasion
d) Comparing the levels of the biomarkers obtained in step a with the levels of the biomarkers obtained in step c, and classifying the treatment as effective if the levels have not risen further over time or have declined with treatment.

### Pharmaceutical Compositions/Routes of Administration/Dosages

While it is possible that for use in therapy, a compound of of the invention as well as pharmaceutically acceptable salts thereof may be administered as the raw chemical, it is common to present the active ingredient as a pharmaceutical composition.

The present invention therefore provides in a further aspect a pharmaceutical composition comprising a compound of formulas (I), (II), (IIa), (IIb), (IIc), and (III) or a pharmaceutically acceptable salt thereof and a pharmaceutically acceptable carrier, diluents or excipient. In one aspect, the invention relates to a pharmaceutical composition comprising a) a compound of compound of formulas (I), (II), (IIa), (IIb), (IIc), and (III) or a pharmaceutically acceptable salt thereof, and b) a pharmaceutically acceptable excipient. The compound of formulas (I), (II), (IIa), (IIb), (IIc), and (III) and pharmaceutically acceptable salts thereof are as described above. The carrier, diluent or excipient must be acceptable in the sense of being compatible with the other ingredients of the composition and not deleterious to the recipient thereof.

In accordance with another aspect of the invention there is also provided a process for the preparation of a pharmaceutical composition including admixing a compound of the formulas (I), (II), (IIa), (IIb), (IIc), and (III) or a pharmaceutically acceptable salt thereof, with a pharmaceutically acceptable carrier, diluent or excipient. The pharmaceutical composition can be for use in the treatment of any of the conditions described herein.

Further provided is a pharmaceutical composition for the treatment of diseases or conditions for which an mTOR kinase inhibitor is indicated, comprising a compound of formulas (I), (II), (IIa), (IIb), (IIc), and (III) or a pharmaceutically acceptable salt thereof.

Further provided is a pharmaceutical composition comprising 0.05 to 1000 mg of a compound of formulas (I), (II), (IIa), (IIb), (IIc), and (III) or a pharmaceutically acceptable salt thereof and 0.1 to 2 g of a pharmaceutically acceptable carrier, diluent or excipient.

Since the compounds of formulas (I), (II), (IIa), (IIb), (IIc), and (III) are intended for use in pharmaceutical compositions it will be readily understood that they are each preferably provided in substantially pure form, for example, at least 60% pure, more suitably at least 75% pure and preferably at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Pharmaceutical compositions may be adapted for administration by any appropriate route, for example by the oral (including buccal or sublingual), rectal, inhaled, intranasal, topical (including buccal, sublingual or transdermal), vagina, ocular or parenteral (including subcutaneous, intramuscular, intravenous or intradermal) route. Such compositions may be prepared by any method known in the art of pharmacy, for example by bringing into association the active ingredient with the carrier or excipient.

In one embodiment the pharmaceutical composition is adapted for oral administration.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine particle size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilising agent such as agaragar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture.

Oral fluids such as solution, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavour additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be prepared as an amorphous molecular dispersion in a polymer matrix, such as hydroxypropylmethyl cellulose acetate succinate, using a spray-dried dispersion (SDD) process to improve the stability and solubility of the drug substance.

The compounds of the invention may also be delivered using a liquid encapsulation technology to improve properties such as bioavailability and stability, in either liquid or semi-solid filled hard capsule or soft gelatin capsule formats.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

In another aspect, the invention is directed to a dosage form adapted for administration to a patient by nasal or inhaled administration, for example, as a dry powder, an aerosol, a suspension, or a solution formulation.

Dry powder formulations for delivery to the lung by inhalation typically comprise a compound of formulas (I), (II), (IIa), (IIb), (IIc), and (III) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (for example micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

The dry powder formulations for use in accordance with the present invention may be administered via inhalation devices. As an example, such devices can encompass capsules and cartridges of for example gelatin, or blisters of, for example, laminated aluminium foil. In various embodiments, each capsule, cartridge or blister may contain doses of formulation according to the teachings presented herein. Examples of inhalation devices may include those intended for unit dose or multi-dose delivery of formulation, including all of the devices set forth herein. As an example, in the case of multi-dose delivery, the formulation can be pre-metered (e.g., as in Diskus®, see GB2242134, U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237) or metered in use (e.g. as in Turbuhaler, see EP 69715, or in the devices described in U.S. Pat. No. 6,321,747). An example of a unit-dose device is Rotahaler (see GB 2064336). In one embodiment, the Diskus® inhalation device comprises an elongate strip formed from a base sheet having a plurality of recesses spaced along its length and a lid sheet peelably sealed thereto to define a plurality of containers, each container having therein an inhalable formulation containing the compound optionally with other excipients and additive taught herein. The peelable seal is an engineered seal, and in one embodiment the engineered seal is a hermetic seal. Preferably, the strip is sufficiently flexible to be wound into a roll. The lid sheet and base sheet will preferably have leading end portions which are not sealed to one another and at least one of the leading end portions is constructed to be attached to a winding means. Also, preferably the engineered seal between the base and lid sheets extends over their whole width. The lid sheet may preferably be peeled from the base sheet in a longitudinal direction from a first end of the base sheet.

A dry powder formulation may also be presented in an inhalation device which permits separate containment of two different components of the formulation, Thus, for example, these components are administrable simultaneously but are stored separately, e.g. in separate pharmaceutical formulations, for example as described in WO 03/061743 A1 WO 2007/012871 A1, WO2007/068896, as well as U.S. Pat. Nos. 8,113,199, 8,161,968, 8,511,304, 8,534,281, 8,746,242 and 9,333,310.

In one embodiment an inhalation device permitting separate containment of components is an inhaler device having two peelable blister strips, each strip containing pre-metered doses in blister pockets arranged along its length, e.g., multiple containers within each blister strip, e.g., ELLIPTA®. Said device has an internal indexing mechanism which, each time the device is actuated, peels open a pocket of each strip and positions the blisters so that each newly exposed dose of each strip is adjacent to the manifold which communicates with the mouthpiece of the device. When the patient inhales at the mouthpiece, each dose is simultaneously drawn out of its associated pocket into the manifold and entrained via the mouthpiece into the patient's respiratory tract. A further device that permits separate containment of different components is DUOHALER™ of Innovata. In addition, various structures of inhalation devices provide for the sequential or separate delivery of the pharmaceutical formulation(s) from the device, in addition to simultaneous delivery.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 200 g-10 mg of the compound of formula (I) or formula (I′) or a pharmaceutically acceptable salt thereof.

Aerosols may be formed by suspending or dissolving a compound of formulas (I), (II), (IIa), (IIb), (IIc), and (III) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formulas (I), (II), (IIa), (IIb), (IIc), and (III) or a pharmaceutically acceptable salt thereof will typically be administered to a patient via a metered dose inhaler (MDI). Such devices are known to those skilled in the art.

A therapeutically effective amount of a compound of formulas (I), (II), (IIa), (IIb), (IIc), and (III) or a pharmaceutically acceptable salt thereof (hereinafter a compound of the invention) will depend upon a number of factors including, for example, the age and weight of the subject, the precise condition requiring treatment and its severity, the nature of the formulation, and the route of administration, and will ultimately be at the discretion of the attendant physician or veterinarian.

In the pharmaceutical composition, each dosage unit for oral or parenteral administration may contain from 0.01 to 3000 mg, or 0.1 to 2000 mg, or more typically 0.5 to 1000 mg of a compound of the invention calculated as the parent compound.

Each dosage unit for nasal or inhaled administration preferably contains from 0.001 to 50 mg, more preferably 0.01 to 5 mg, yet more preferably 1 to 50 mg, of a compound of the invention.

For administration of a nebulised solution or suspension, a dosage unit typically contains from 1 to 15 mg which may suitably be delivered once daily, twice daily or more than twice daily. The compound of the invention may be provided in a dry or lyophilised powder for reconstitution in the pharmacy or by the patient, or may, for example, be provided in an aqueous saline solution.

The compounds of the invention can be administered in a daily dose (for an adult patient) of, for example, an oral or parenteral dose of 0.01 mg to 3000 mg per day, or 0.5 to 1000 mg per day or 0.5 to 300 mg per day, or 2 to 300 mg per day, or a nasal or inhaled dose of 0.001 to 50 mg per day or 0.01 to 50 mg per day, or 1 to 50 mg per day, of the compound of the invention. This amount may be given in a single dose per day or more usually in a number (such as two, three, four, five or six) of sub-doses per day such that the total daily dose is the same. An effective amount of a salt thereof may be determined as a proportion of the effective amount of the compound of formulas (I), (II), (IIa), (IIb), (IIc), and (III) per se.

The compounds of the invention may be employed alone or in combination with other therapeutic agents. Combination therapies according to the present invention thus comprise the administration of at least one compound of formulas (I), (II), (IIa), (IIb), (IIc), and (III) or a pharmaceutically acceptable salt thereof, and the use of at least one other pharmaceutically active agent. Preferably, combination therapies according to the present invention comprise the administration of at least one compound of formulas (I), (II), (IIa), (IIb), (IIc) and (III), or a pharmaceutically acceptable salt thereof, and at least one other pharmaceutically active agent. The compound(s) of the invention and the other pharmaceutically active agent(s) may be administered together in a single pharmaceutical composition or separately and, when administered separately this may occur simultaneously or sequentially in any order. The amounts of the compound(s) of the invention and the other pharmaceutically active agent(s) and the relative timings of administration will be selected in order to achieve the desired combined therapeutic effect.

Thus in a further aspect, there is provided a combination comprising a compound of the invention and at least one other pharmaceutically active agent.

Thus in one aspect, the compound and pharmaceutical compositions according to the invention may be used in combination with or include one or more other therapeutic agents, including therapies for allergic disease, inflammatory disease, autoimmune disease, anti-fibrotic therapies and therapies for obstructive airway disease, therapies for diabetes and related diseases, ocular diseases, and therapies for corneal scarring, corneal injury and corneal wound healing.

Anti-allergic therapies include antigen immunotherapy (such as components and fragments of bee venom, pollen, milk, peanut, CpG motifs, collagen, other components of extracellular matrix which may be administered as oral or sublingual antigens), anti-histamines (such as cetirizine, loratidine, acrivastine, fexofenidine, chlorphenamine), and corticosteroids (such as fluticasone propionate, fluticasone furoate, beclomethasone dipropionate, budesonide, ciclesonide, mometasone furoate, triamcinolone, flunisolide, prednisolone, hydrocortisone).

Anti-inflammatory therapies include NSAIDs (such as aspirin, ibuprofen, naproxen), leukotriene modulators (such as montelukast, zafirlukast, pranlukast), and other anti-inflammatory therapies (such as iNOS inhibitors, tryptase inhibitors, IKK2 inhibitors, p38 inhibitors (losmapimod, dilmapimod), elastase inhibitors, beta2 agonists, DPi antagonists, DP2 antagonists, pI3K delta inhibitors, ITK inhibitors, LP (lysophosphatidic) inhibitors or FLAP (5-lipoxygenase activating protein) inhibitors (such as sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate); adenosine a2a agonists (such as adenosine and regadenoson), chemokine antagonists (such as CCR3 antagonists or CCR4 antagonists), mediator release inhibitors.

Therapies for autoimmune disease include DMARDS (such as methotrexate, leflunomide, azathioprine), biopharmaceutical therapies (such as anti-IgE, anti-TNF, anti-interleukins (such as anti-IL-1, anti-IL-6, anti-IL-12, anti-IL-17, anti-IL-18), receptor therapies (such as etanercept and similar agents); antigen non-specific immunotherapies (such as interferon or other cytokines/chemokines, cytokine/chemokine receptor modulators, cytokine agonists or antagonists, TLR agonists and similar agents).

Other anti-fibrotic therapies includes inhibitors of TGFβ synthesis (such as pirfenidone), tyrosine kinase inhibitors targeting the vascular endothelial growth factor (VEGF), platelet-derived growth factor (PDGF) and fibroblast growth factor (FGF) receptor kinases (such as Nintedanib (BIBF-1120) and imatinib mesylate (Gleevec)), endothelin receptor antagonists (such as ambrisentan or macitentan), antioxidants (such as N-acetylcysteine (NAC); broad-spectrum antibiotics (such as cotrimoxazole, tetracyclines (minocycline hydrochloride)), phosphodiesterase 5 (PDE5) inhibitors (such as sildenafil), anti-avpx antibodies and drugs (such as anti-αvβ6 monoclonal antibodies such as those described in WO2003100033A2 may be used in combination, intetumumab, cilengitide) may be used in combination.

Therapies for obstructive airway diseases include bronchodilators such as short-acting P2-agonists, such as salbutamol), long-acting P2-agonists (such as salmeterol, formoterol and vilanterol), short-acting muscarinic antagonists (such as ipratropium bromide), long-acting muscarinic antagonists, (such as tiotropium, umeclidinium).

In some embodiments, treatment can also involve combination of a compound of this invention with other existing modes of treatment, for example existing agents for treatment of diabetic ocular diseases, such as anti VEGF therapeutics e.g. Lucentis, Avastin, and Aflibercept and steroids, e.g., triamcinolone, and steroid implants containing fluocinolone acetonide.

In some embodiments, treatment can also involve combination of a compound of this invention with other existing modes of treatment, for example existing agents for treatment of corneal scarring, corneal injury or corneal wound healing, such as Gentel, calf blood extract, Levofloxacin, and Ofloxacin.

The compounds and compositions of the invention may be used to treat cancers alone or in combination with cancer therapies including chemotherapy, radiotherapy, targeted agents, immunotherapy and cell or gene therapy.

Rapamycin (sirolimus) and analogues of rapamycin (everolimus, ridaforolimus, temsirolimus, zotarolimus) may be used in combination with mTOR kinase inhibitors to augment mTOR modulation as described for an everolimus combination with a pan PI3k/mTOR inhibitor (Nyfeler et al., 2012)

Therefore, in one embodiment, there is provided a combination of
a) a compound or pharmaceutically acceptable salt of the invention; and
b) Rapamycin or an analogue of rapamycin.

In another embodiment, there is provided a combination of
a) a compound or pharmaceutically acceptable salt of the invention; and
b) A compound selected from the group consisting of Sirolimus, Everolimus, Ridaforolimus, Temsirolimus, Zotarolimus and pharmaceutically acceptable salts thereof.

In one embodiment there is provided a composition comprising
a) a compound or pharmaceutically acceptable salt of the invention; and
b) Rapamycin or an analogue of rapamycin.

In another embodiment, there is provided a composition comprising
a) a compound or pharmaceutically acceptable salt of the invention; and
b) a compound selected from the group consisting of Sirolimus, Everolimus, Ridaforolimus, Temsirolimus, Zotarolimus and pharmaceutically acceptable salts thereof.

In one embodiment there is provided a method for treatment of idiopathic pulmonary fibrosis in a human in need thereof comprising administering to said human a therapeutically effective amount of:
a) a compound or pharmaceutically acceptable salt of the invention; and
b) Rapamycin or an analogue of rapamycin.

In another embodiment there is provided a method for treatment of idiopathic pulmonary fibrosis in a human in need thereof comprising administering to said human a therapeutically effective amount of:
a) a compound or pharmaceutically acceptable salt of the invention; and
b) A compound selected from the group consisting of Sirolimus, Everolimus, Ridaforolimus, Temsirolimus, Zotarolimus and pharmaceutically acceptable salts thereof.

The combinations referred to above may conveniently be presented for use in the form of a pharmaceutical composition and thus pharmaceutical compositions comprising a combination as defined above together with a pharmaceutically acceptable diluent or carrier represent a further aspect of the invention. The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

It will be appreciated that when the compound of the present invention is administered in combination with one or more other therapeutically active agents normally administered by the inhaled, intravenous, oral, intranasal, ocular topical or other route that the resultant pharmaceutical composition may be administered by the same route. Alternatively, the individual components of the composition may be administered by different routes.

### General Synthetic Routes

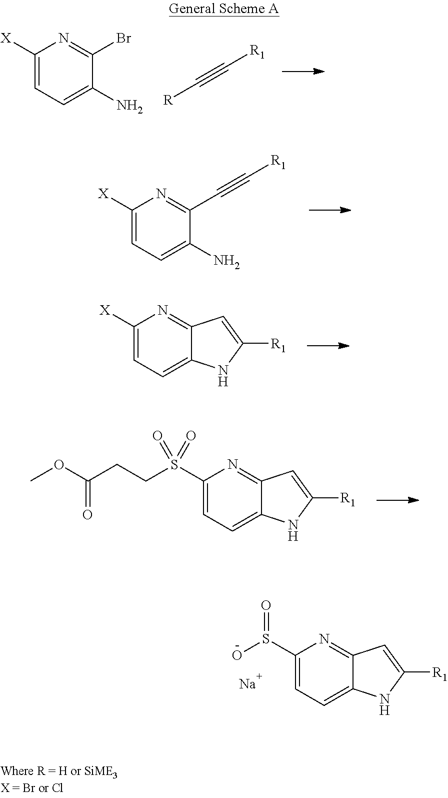

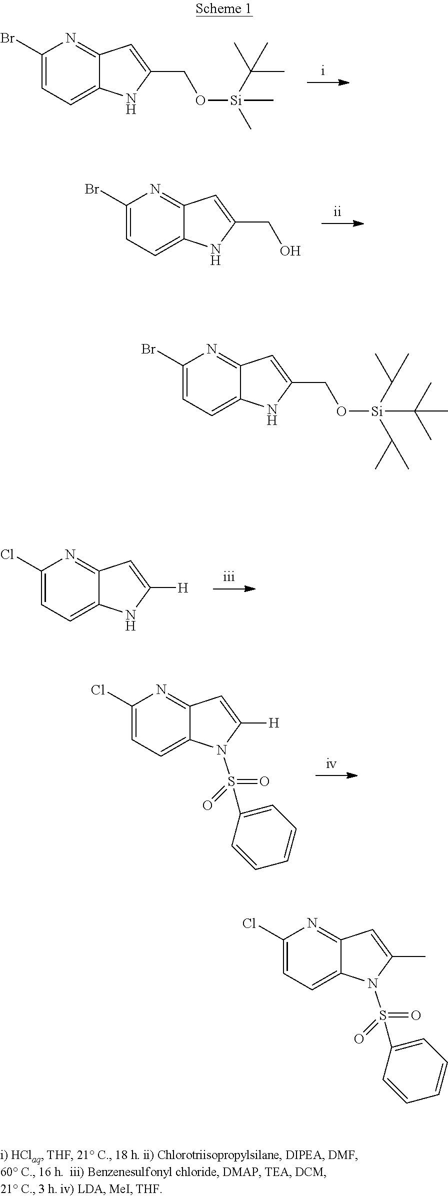 [SMILES: CC(C)(C)[Si](C)(C)OCc1cc2nc(Br)ccc2[nH]1 ; OCc1cc2nc(Br)ccc2[nH]1 ; CC(C)[Si](OCc1cc2nc(Br)ccc2[nH]1)(C(C)C)C(C)(C)C ; Clc1ccc2[nH]ccc2n1 ; O=S(=O)(c1ccccc1)n1ccc2nc(Cl)ccc21 ; Cc1cc2nc(Cl)ccc2n1S(=O)(=O)c1ccccc1]

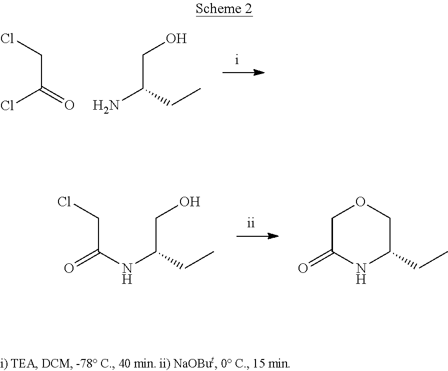

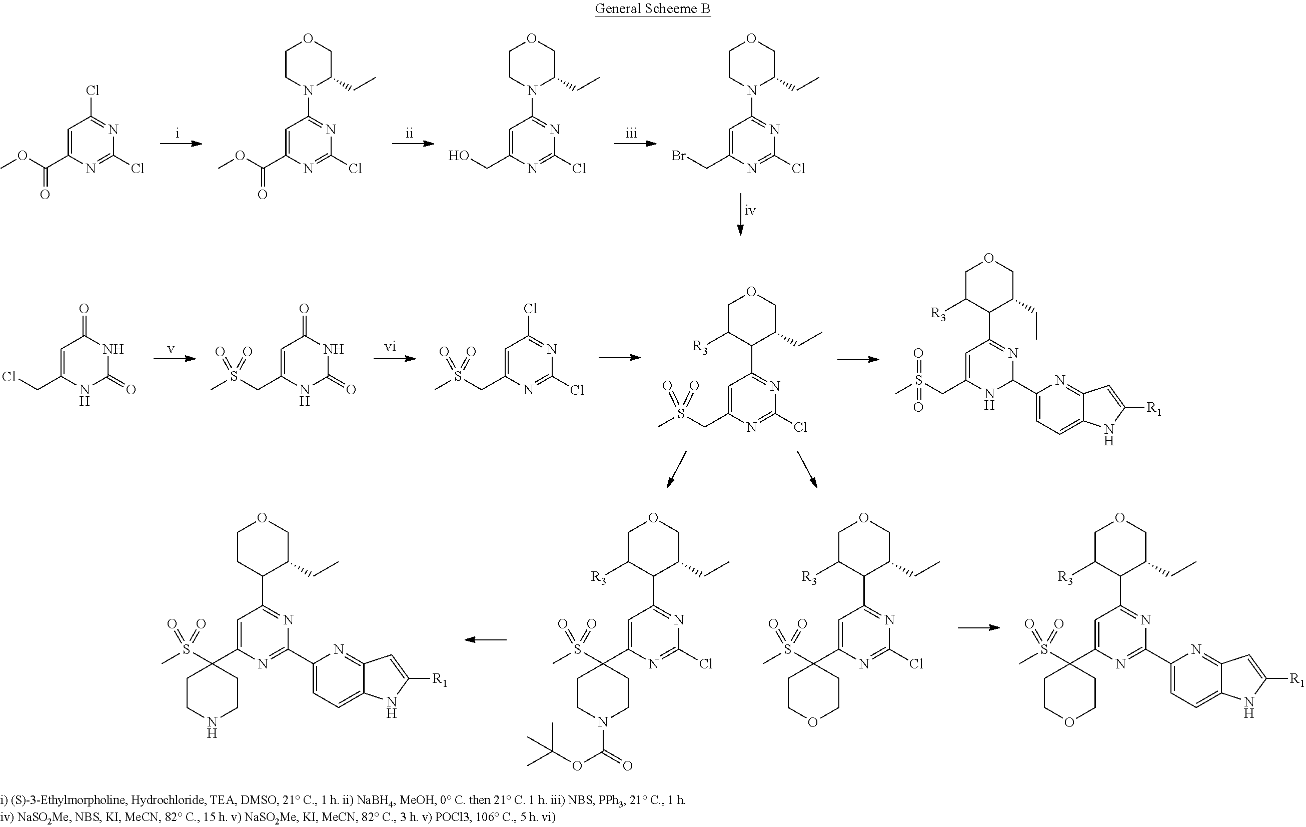

General Scheme A

Sonagashira reaction of a suitably substituted 2-bromo-3-aminopyridine with an appropriate acetylene followed by treatment with base may be used to prepare the 4-aza-indole. Reaction of this azaindole with Sodium 1-methyl 3-sulfinopropanoate in a copper mediated cross coupling affords the sulfonyl propionate which may be decarboxylated in the presence of base to liberate the sodium sulfinate.

General Scheme B

S_{N}Ar reactions of a suitably substituted morpholine on a dihalopyrimidine may be used to prepare suitably substituted 6-(methylsulphonyl)methyl pyrimidines, which may be optionally further modified with cyclic ethers or amines in the presence of strong base such as sodium tert-butoxide. A desulfinative coupling with an appropriate 4-aza-indole sulfinate may be used to prepare the final molecules.

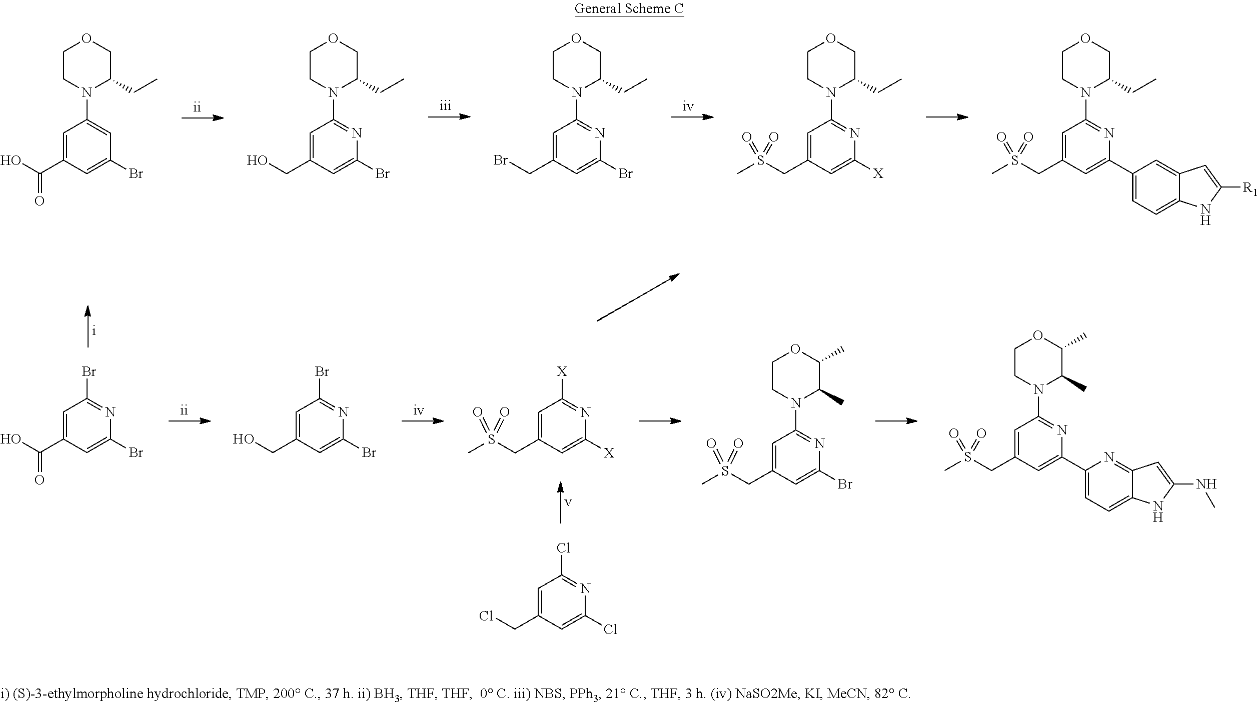

General Scheme C

S_{N}Ar reactions of a suitably substituted morpholine on a dihalopyrimidine may be used to prepare suitably substituted 6-(methylsulphonyl)methyl pyrimidines. A desulfinative coupling with an appropriate 4-aza-indole sulfinate may be used to prepare the final molecules.

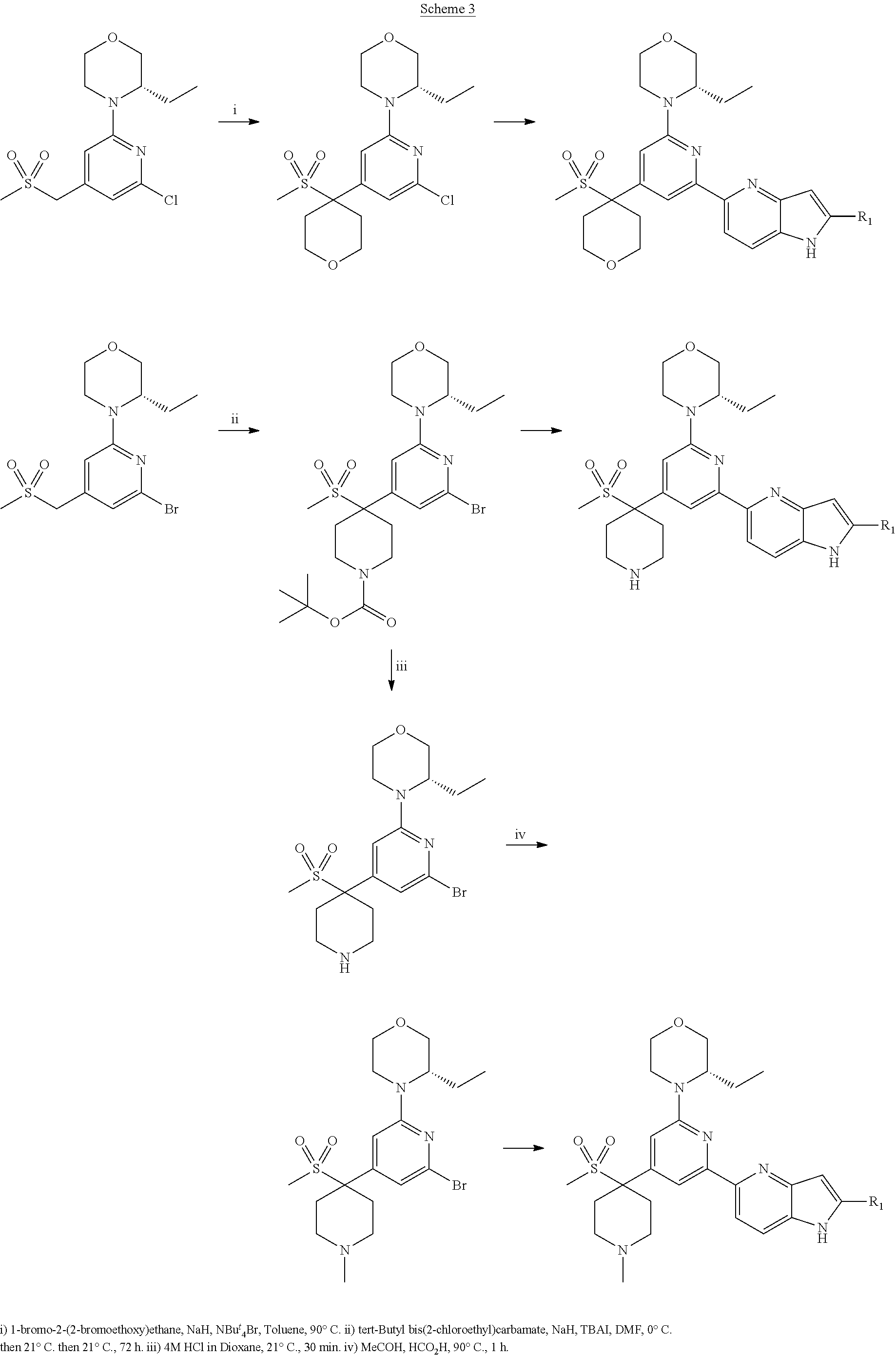

Scheme 3 shows a desulfinative coupling may be used to prepare final molecules from a suitably substituted 6-(methylsulphonyl)methyl pyridine and an appropriate 4-aza-indole sulfinate.

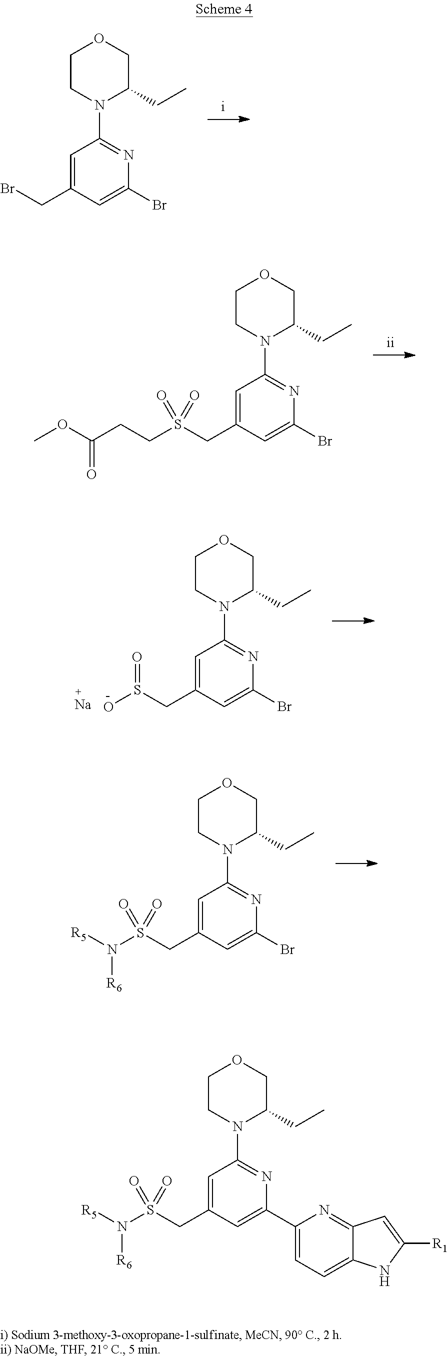

Scheme 4 shows sodium (S)-(2-bromo-6-(3-ethylmorpholino)pyridin-4-yl)methanesulfinate may be reacted with a variety of amines followed by a desulfinative coupling with an appropriate 4-aza-indole sulfinate to prepare final sulphonamide molecules.

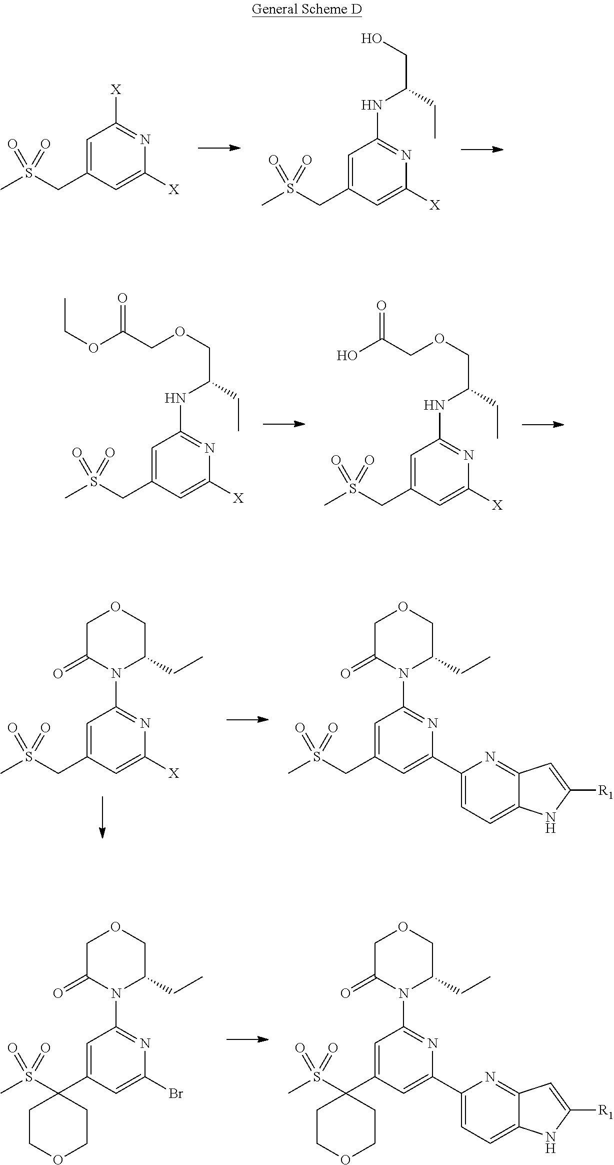

General Scheme D

An S_{N}Ar reaction between a 2,6-dihalo-4-((methylsulfonyl)methyl)pyridine and (S)-2-aminobutan-1-ol, followed by reaction with ethyl diazoacetate and subsequent hydrolysis with sodium hydroxide and ring closure using HATU and DMF may be used to prepare halogenated 5(S)-ethylmorpholin-3-one pyridines which may be optionally substituted with cyclic ethers (as described in Scheme 3). A desulfinative coupling with an appropriate 4-aza-indole sulfinate to prepare final morpholinone molecules.

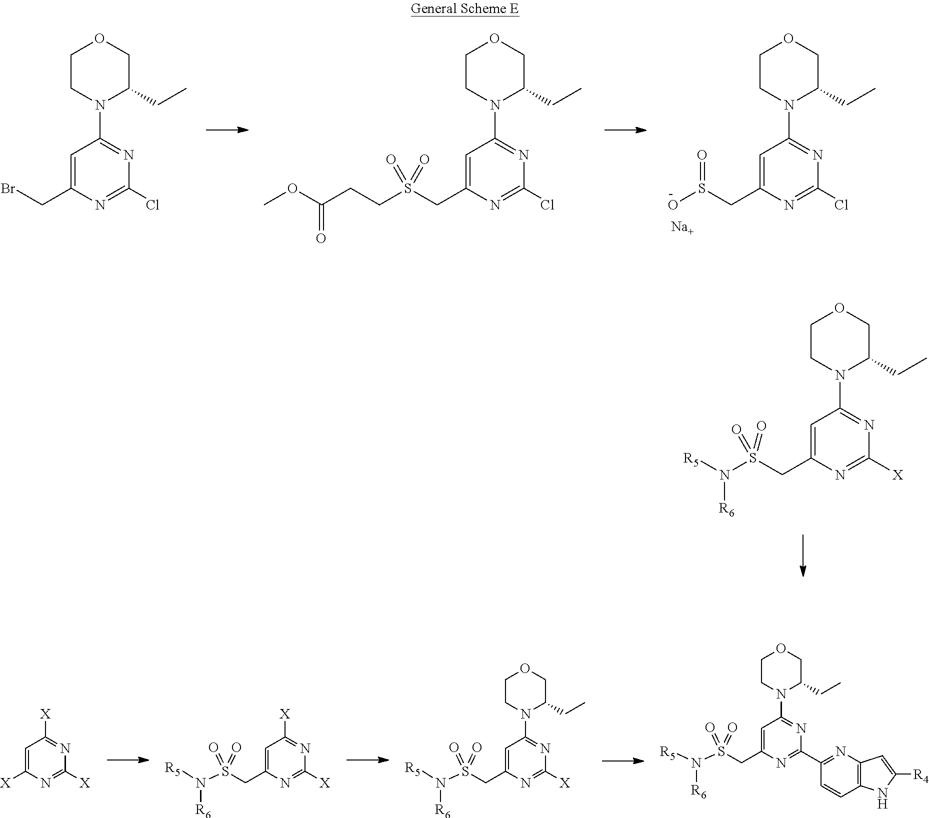

Suitably substituted 2-halo-pyrimidine sulphonamides may either be prepared as previously described in Scheme 4 or by reaction of a suitably substituted methanesulphonamide with a suitably substituted trihalo pyrimidine in the presence of butyl lithium followed by an S_{N}Ar with 3(S)-ethylmorpholine hydrochloride. A subsequent desulfinative coupling with an appropriate 4-aza-indole sulfinate may be used to prepare final sulphonamide molecules.

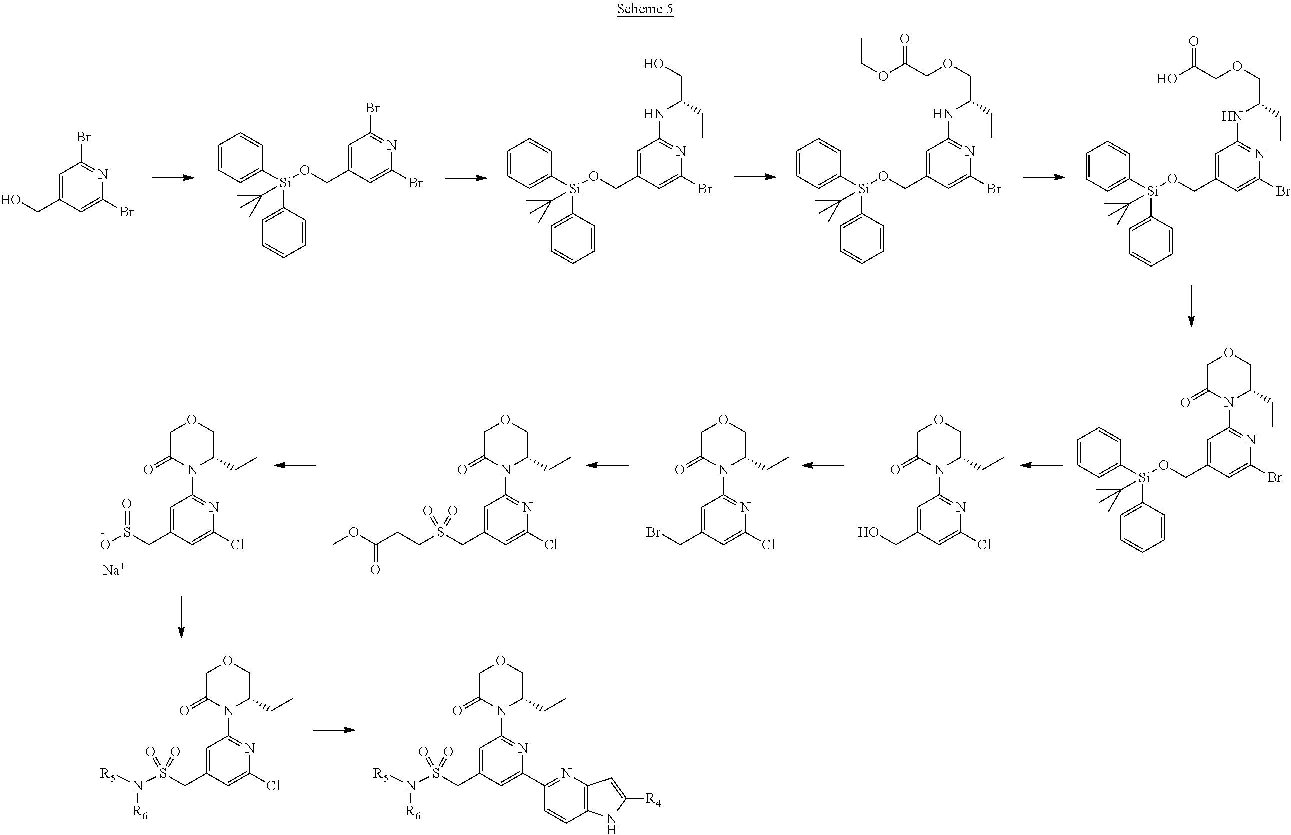

Scheme 5

5-Oxomorpholinopyridine sulphonamides may be prepared using methodology previously described in General schemes D and Scheme 4. A final desulfinative coupling with an appropriate 4-aza-indole sulfinate may be use to prepare final morpholinone sulphonamide molecules.

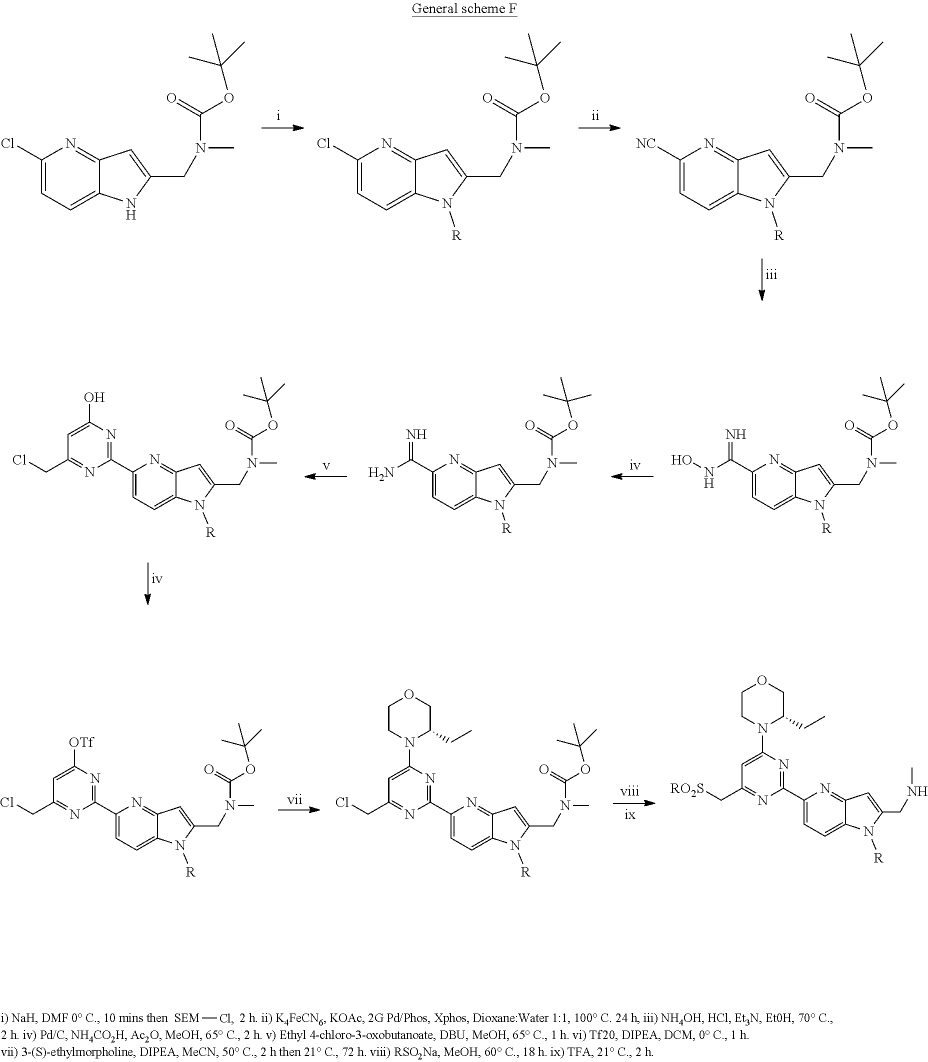

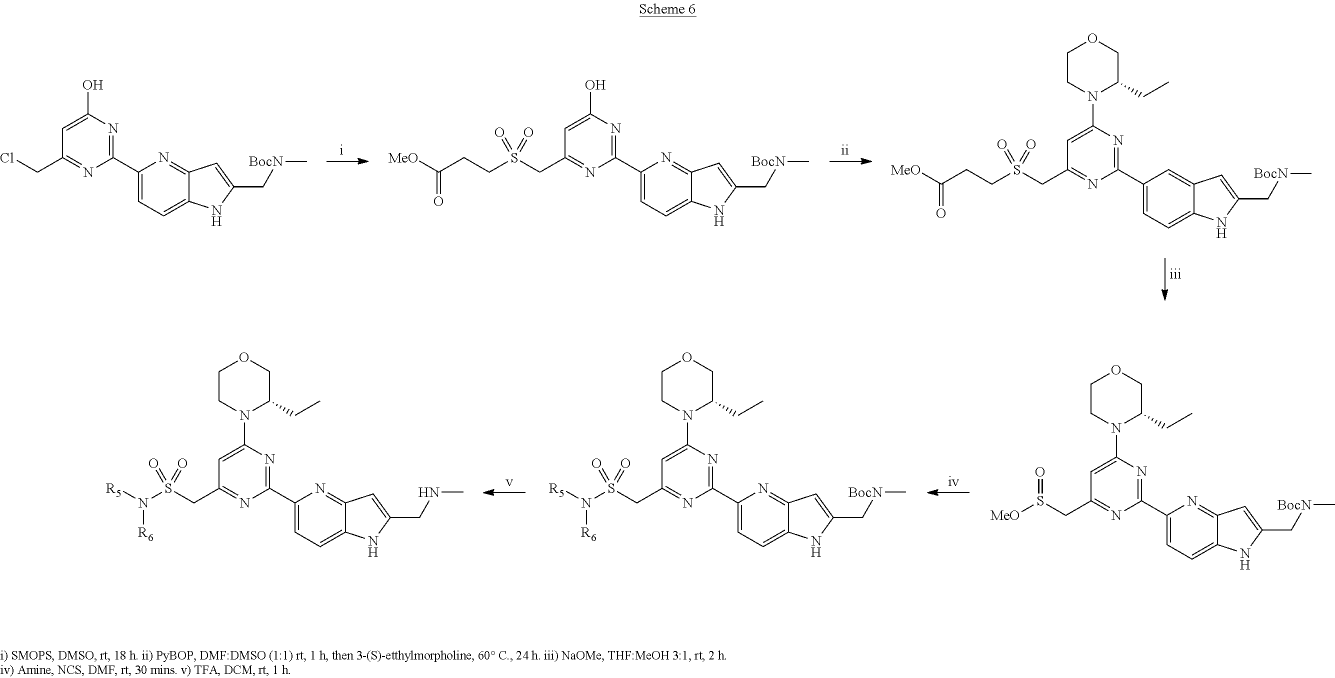

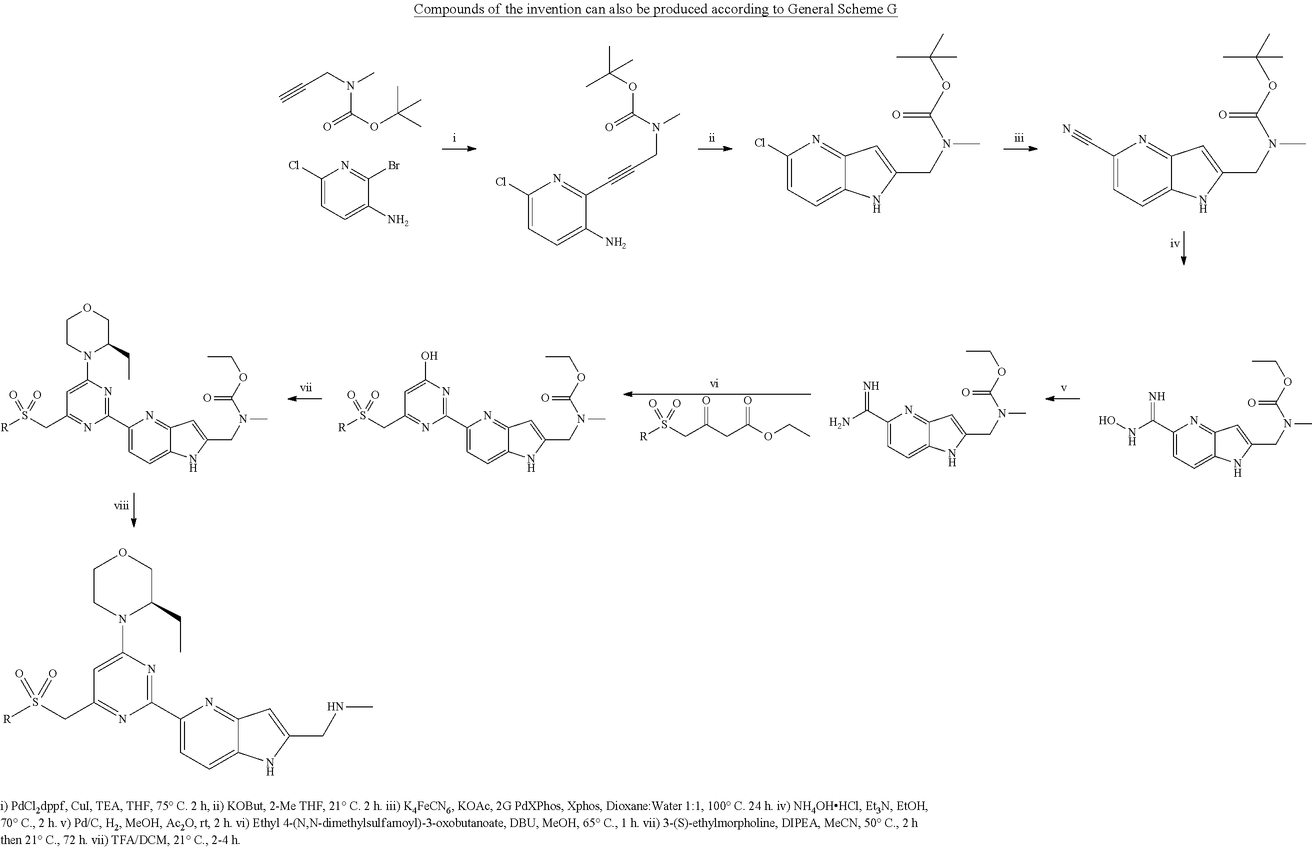

### Abbreviations

The following list provides definitions of certain abbreviations as used herein. It will be appreciated that the list is not exhaustive, but the meaning of those abbreviations not herein below defined will be readily apparent to those skilled in the art.

Ac (acetyl)
Bu (butyl)
Chiralcel OD-H (cellulose tris(3,5-dimethylphenylcarbamate) coated on 5 m silica gel)
Chiralpak AD-H (amylose tris(3,5-dimethylphenylcarbamate) coated on 5 m silica gel)
Chiralpak ID (amylose tris(3-chlorophenylcarbamate) immobilised on 5 m silica gel)
Chiralpak AS (amylose tris((5)-alpha-methylbenzylcarbamate) coated on 5 m silica gel)

### CSH (Charged Surface Hybrid Technology)

CV (column volume)
DCM (dichloromethane)

### DMF (N,N-dimethylformamide)

DMSO (dimethylsulfoxide)
Et (ethyl)
EtOH (ethanol)
EtOAc (ethyl acetate)
h or hr (hour/hours)
MDAP (mass directed auto-preparative HPLC)
Me (methyl)
MeOH (methanol)
Mg₂SO₄ (Magnesium Sulphate)
min (minute/minutes)
Pd(dppf)Cl₂ (1,1′-[bis(diphenylphosphino)ferrocene]dichloropalladium (II))

### Pet Ether (Petroleum Ether)

Ph (phenyl)
ⁱPr (isopropyl)
room temp (room temperature)

### Si (Silica)

SPE (solid phase extraction)
TBME (tert-butyl methyl ether)
TEA (triethylamine)
TFA (trifluoroacetic acid)
THF (tetrahydrofuran)
TLC (thin layer chromatography)

### UPLC (Ultra Performance Liquid Chromatography)

References to brine refer to a saturated aqueous solution of sodium chloride.

### Experimental Details

Analytical LCMS

Analytical LCMS was conducted on one of the following systems A, B, C or D. The UV detection to all systems was an averaged signal from wavelength of 220 nm to 350 nm and mass spectra were recorded on a mass spectrometer using alternate-scan positive and negative mode electrospray ionization.

LCMS purity is derived from diode array detection.

Experimental details of LCMS systems A-B as referred to herein are as follows:

System A
Column: 50 mm×2.1 mm ID, 1.7 m Acquity UPLC BEH C₁₈ column
Flow Rate: 1 mL/min.
Temp.: 40° C.
Solvents: A: 10 mM ammonium bicarbonate in water adjusted to pH10 with ammonia solution
   B: Acetonitrile

| Gradient: | Time (min) | A % | B % |
|---|---|---|---|
| | 0 | 99 | 1 |
| | 1.5 | 3 | 97 |
| | 1.9 | 3 | 97 |
| | 2.0 | 99 | 1 |

System B

Column: 50 mm×2.1 mm ID, 1.7 m Acquity UPLC BEH C18 column
Flow Rate: 1 mL/min

### Temp.: 40° C.

Solvents: A: 0.1% v/v solution of formic acid in water
   B: 0.1% v/v solution of formic acid in acetonitrile

| Gradient: | Time (min) | A % | B % |
|---|---|---|---|
| | 0 | 97 | 3 |
| | 1.5 | 0 | 100 |
| | 1.9 | 0 | 100 |
| | 2.0 | 97 | 3 |

System C

Column: 30 mm×2.1 mm ID, 1.7 m Kinetex XB-C18 C18 column
Flow Rate: 1 mL/min

### Temp.: 40° C.

Solvents: A: 0.05% v/v solution of TFA in water
   B: 0.05% v/v solution of TFA in acetonitrile

| Gradient: | Time (min) | A % | B % |
|---|---|---|---|
| | 0 | 95 | 5 |
| | 0.6 | 5 | 95 |
| | 1 | 5 | 95 |
| | 1.05 | 95 | 5 |

System D

Column: 50 mm×2.1 mm ID, 1.7 m Acquity UPLC BEH C18 column
Flow Rate: 0.6 mL/min

### Temp.: 35° C.

Solvents: A: 0.1% v/v solution of formic acid in water
   B: 0.1% v/v solution of formic acid in acetonitrile

| Gradient: | Time (min) | A % | B % |
|---|---|---|---|
| | 0 | 97 | 3 |
| | 0.4 | 97 | 3 |
| | 7.5 | 2 | 98 |
| | 9.5 | 2 | 98 |
| | 9.6 | 97 | 3 |

### Mass Directed Auto-Preparative HPLC

Crude products were purified by MDAP HPLC by one of the following methods. The run time was 15 min unless otherwise stated. The UV detection for all methods was an averaged signal from wavelength of 210 nm to 350 nm and mass spectra were recorded on a mass spectrometer using alternate-scan positive and negative mode electrospray ionization.

Method HPH_Meth_B:

Method HPH_Meth_B was conducted on an XBridge C₁₈ column (typically 100 mm×30 mm i.d. 5 μm packing diameter) at ambient temperature. The solvents employed were:
A=10 mM aqueous ammonium bicarbonate adjusted to pH 10 with ammonia solution.
B=acetonitrile.
The gradient employed was:

| Time (min) | Flow Rate (mL/min) | % A | % B |
|---|---|---|---|
| 0 | 40 | 85 | 15 |
| 1 | 40 | 85 | 15 |
| 20 | 40 | 45 | 55 |
| 21 | 40 | 1 | 99 |
| 25 | 40 | 1 | 99 |

### Method HPH_Meth_C:

Method HPH_Meth_C was conducted on an XBridge C₁₈ column (typically 100 mm×30 mm i.d. 5 μm packing diameter) at ambient temperature. The solvents employed were:
A=10 mM aqueous ammonium bicarbonate adjusted to pH 10 with ammonia solution.
B=acetonitrile.
The gradient employed was:

| Time (min) | Flow Rate (mL/min) | % A | % B |
|---|---|---|---|
| 0 | 40 | 70 | 30 |
| 1 | 40 | 70 | 30 |
| 10 | 40 | 15 | 85 |
| 11 | 40 | 1 | 99 |
| 15 | 40 | 1 | 99 |

### Method HPH_Meth_EXT_C:

Method EXT_C was conducted on an XBridge C₁₈ column (typically 100 mm×30 mm i.d. 5 μm packing diameter) at ambient temperature. The solvents employed were:
A=10 mM aqueous ammonium bicarbonate adjusted to pH 10 with ammonia solution.
B=acetonitrile.
The gradient employed was:

| Time (min) | Flow Rate (mL/min) | % A | % B |
|---|---|---|---|
| 0 | 40 | 70 | 30 |
| 1 | 40 | 70 | 30 |
| 20 | 40 | 15 | 85 |
| 20.5 | 40 | 1 | 99 |
| 25 | 40 | 1 | 99 |

### Intermediates:

### Intermediate 1 tert-butyl N-methyl-N-(prop-2-yn-1-yl)carbamate

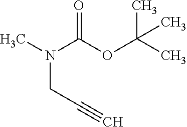

To tert-butyl prop-2-yn-1-ylcarbamate (150.00 g, 967 mmol) in Tetrahydrofuran (THF) (2400 mL) stirred at 0° C. was added sodium hydride (46.4 g, 1160 mmol). The reaction mixture was stirred at 0° C. for 30 minutes. Then iodomethane (274 g, 1933 mmol) was added dropwise. The resulting mixture was allowed to warm to room temperature and stirred for overnight.

The reaction was quenched by addition of water slowly (1500 mL). Then Tetrahydrofuran was removed in vacuo and the residue was extracted with EtOAc (2000 mL×3). The combine organic layers were dried over Na2SO4, filtered and concentrated. This gave yellow oil.

Tert-butyl methyl(prop-2-yn-1-yl)carbamate, (160 g, 936 mmol, 97% yield) was isolated as yellow oil; 160 g, 97%.

¹H NMR (400 MHz, DMSO-d₆) δ 3.98 (s, 2H), 3.17 (s, 1H), 2.80 (s, 3H), 1.36-1.44 (bs, 9H)

### Intermediate 2 tert-butyl N-[3-(3-amino-6-bromopyridin-2-yl)prop-2-yn-1-yl]-N-methylcarbamate

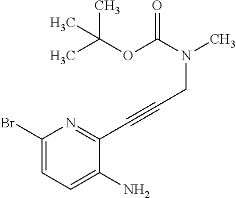 [SMILES: CN(CC#Cc1nc(Br)ccc1N)C(=O)OC(C)(C)C]

Triethylamine (332 mL, 2382 mmol) was added to tert-butyl methyl(prop-2-yn-1-yl)carbamate (81 g, 476 mmol), 2,6-dibromopyridin-3-amine (60 g, 238 mmol) and PdCl2(PPh3)2 (16.72 g, 23.82 mmol) in Tetrahydrofuran (THF) (1200 mL). The reaction was mixture degassed under a flow of nitrogen and stirred at 80° C. for 4 hours.

The reaction mixture was combined with previous batches of 4 mmol and 159 mmol. The combined mixture was filtered and the filtrate partitioned between EtOAc (2500 mL) and water (2500 mL), extracted with ethyl acetate (1500 mL×3). The organic phase was washed with saturated brine (1000 mL), dried over sodium sulphate and evaporated in vacuo to give the crude product as a yellow solid.

The sample was preabsorbed on silica and purified on silica (Si) 1000 g using a 0-30% ethyl acetate-petroleum ether over 180 min, flow rate 180 mL/min. The appropriate fractions were identified by UV absorbance (340 nm), combined and evaporated in vacuo to give the required product tert-butyl (3-(3-amino-6-bromopyridin-2-yl)prop-2-yn-1-yl)(methyl)carbamate (70 g, 202 mmol, 51% yield) as a yellow solid

LCMS (System C, UV, ESI): Rt=0.80 min, [M+H]⁺ 340, 342

### Intermediate 3 tert-butyl N-({5-bromo-1H-pyrrolo[3,2-b]pyridin-2-yl}methyl)-N-methylcarbamate

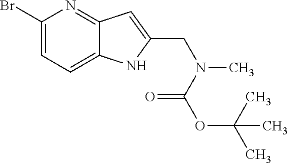 [SMILES: CN(Cc1cc2nc(Br)ccc2[nH]1)C(=O)OC(C)(C)C]

To tert-butyl (3-(3-amino-6-bromopyridin-2-yl)prop-2-yn-1-yl)(methyl)carbamate (70 g, 206 mmol) in Tetrahydrofuran (THF) (700 mL) stirred under nitrogen at room temperature was added sodium 2-methylpropan-2-olate (21.75 g, 226 mmol). The reaction mixture was stirred at room temperature for 2 hours. The reaction mixture was partitioned between ethyl acetate (3000 mL) and water (2000 mL), extracted with ethyl acetate (1000 mL×3). The organic phase was washed with saturated brine (700 mL), dried over sodium sulphate and evaporated in vacuo to give the crude product as a brown solid.

The sample was preabsorbed on silica and purified on silica (Si) 660 g using a 0-50% ethyl acetate-petroleum ether over 120 mins, flow rate 100 mL/min. The appropriate fractions were identified by UV absorbance (300 nm), combined and evaporated in vacuo to give the required product tert-butyl ((5-bromo-1H-pyrrolo[3,2-b]pyridin-2-yl)methyl)(methyl)carbamate (54.0763 g, 60.6 mmol, 29.5% yield) as a light yellow solid.

¹H-NMR (400 MHz, DMSO-d6): δ [ppm] 11.49 (s, 1H), 7.69 (d, J=8.4 Hz, 1H), 7.20 (d, J=8.4 Hz, 1H), 6.35 (s, 1H), 4.54 (s, 2H), 2.84 (s, 3H), 1.42 (s, 9H).

LCMS (System C, UV, ESI): Rₜ=1.37 min, [M+H]⁺ 340, 342

### Intermediate 4 methyl 3-{[2-({[(tert-butoxy)carbonyl](methyl)amino}methyl)-1H-pyrrolo[3,2-b]pyridin-5-yl]sulfonyl}propanoate

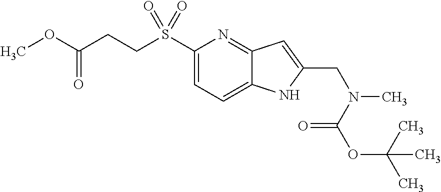 [SMILES: COC(=O)CCS(=O)(=O)c1ccc2[nH]c(CN(C)C(=O)OC(C)(C)C)cc2n1]

To tert-butyl ((5-bromo-1H-pyrrolo[3,2-b]pyridin-2-yl)methyl) (methyl)carbamate (12.3 g, 36.2 mmol) and sodium 3-methoxy-3-oxopropane-1-sulfinate (12.59 g, 72.3 mmol) in Dimethyl Sulfoxide (DMSO) (180 mL) stirred under nitrogen was added copper(I) iodide (6.89 g, 36.2 mmol). The reaction mixture was stirred under nitrogen at 110° C. for 2 hours.

The reaction mixture was added EtOAc (500 mL) and filtered and the filtrate was washed with a mixture of water/aqueous saturated ammonium chloride/aqueous saturated sodium bicarbonate (4:1:1) (1000 mL), water (400 mL) and saturated brine (300 mL), dried over sodium sulphate and evaporated in vacuo to give the crude product as an orange solid.

The sample was loaded in dichloromethane and purified on silica (Si) 330 g using a 0-50% ethyl acetate-petroleum ether over 60 mins, flow rate 100 mL/min. The appropriate fractions were identified by UV absorbance (292 nm), combined and evaporated in vacuo to give the required product methyl 3-((2-(((tertbutoxycarbonyl)(methyl)amino)methyl)-1H-pyrrolo[3,2-b]pyridin-5-yl)sulfonyl)propanoate (3.2352 g, 7.71 mmol, 21.33% yield) as an off-white solid.

1H NMR (400 MHz, DMSO-d6) δ 11.87 (s, 1H), 8.01-7.99 (m, 1H), 7.74 (d, J=8.8 Hz, 1H), 6.56 (s, 1H), 4.61 (s, 2H), 3.70-3.65 (m, 2H), 3.52 (s, 3H), 2.89 (s, 3H), 2.69 (t, J=7.2 Hz, 2H), 1.44 (s, 9H).

LCMS (System C, UV, ESI): Rₜ=1.24 min, [M+H]⁺ 412

### Intermediate 5 5-bromo-2-(((tert-butyldimethylsilyl)oxy)methyl)-1Hpyrrolo[3,2-b]pyridine

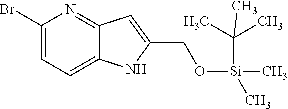 [SMILES: CC(C)(C)[Si](C)(C)OCc1cc2nc(Br)ccc2[nH]1]

To 2,6-dibromopyridin-3-amine (40.00 g, 159 mmol) and tert-butyldimethyl(prop-2-yn-1-yloxy)silane (38.6 ml, 191 mmol) in pyrrolidine (104 ml, 1270 mmol) stirred under nitrogen at room temperature was added bis(triphenylphosphine)palladium(II) chloride (5.57 g, 7.94 mmol). The reaction mixture was stirred at 60° C. for 4 hr.

The reaction mixture was evaporated and the residue partitioned between EtOAc (1000 mL) and water (1000 mL), extracted with EtOAc (1000 mL). The organic phase was washed saturated brine (500 mL), dried over sodium sulphate and evaporated in vacuo to give the crude product as a yellow solid.

The sample was preabsorbed on silica and purified on silica (Si) 660 g using a 0-15% ethyl acetate-petroleum ether over 80 mins, flow rate 100 mL/min. The appropriate fractions were identified by UV absorbance (300 nm), combined and evaporated in vacuo to give 5-bromo-2-(((tert-butyldimethylsilyl)oxy)methyl)-1Hpyrrolo[3,2-b]pyridine (12 g, 32.3 mmol, 20.37% yield) as a yellow solid.

LCMS (System C, UV, ESI): Rₜ=1.199 min, [M+H]⁺ 341, 343

### Intermediate 6 {5-bromo-1H-pyrrolo[3,2-b]pyridin-2-yl}methanol

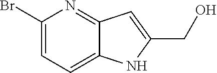 [SMILES: OCc1cc2nc(Br)ccc2[nH]1]

To a solution of 5-bromo-2-(((tert-butyldimethylsilyl)oxy)methyl)-1H-pyrrolo[3,2-b]pyridine (12.00 g, 35.2 mmol) in Tetrahydrofuran (THF) (648 mL) stirred at room temperature was added a solution of hydrogen chloride (0.128 g, 3.52 mmol) in Water (72 mL) dropwise. The reaction mixture was stirred at room temperature for overnight.

The reaction mixture was evaporated to give the crude product.

The sample was preabsorbed on silica and purified on silica (Si) 330 g using a 0-60% ethyl acetate-petroleum ether over 80 mins, flow rate 100 mL/min. The appropriate fractions were identified by UV absorbance (300 nm), combined and evaporated in vacuo to give (5-bromo-1H-pyrrolo[3,2-b]pyridin-2-yl)methanol (5.0362 g, 21.40 mmol, 60.9% yield) as a yellow solid.

1H NMR (400 MHz, DMSO-d6) δ 11.53 (s, 1H), 7.65 (dd, J=8.4, 0.9 Hz, 1H), 7.17 (d, J=8.4 Hz, 1H), 6.38 (dd, J=2.0, 1.0 Hz, 1H), 5.48 (t, J=5.6 Hz, 1H), 4.66 (dd, J=5.7, 0.8 Hz, 2H).

LCMS (System C, UV, ESI): Rₜ=0.865 min, [M+H]⁺ 227

### Intermediate 7 5-bromo-2-({[tris(propan-2-yl)silyl]oxy}methyl)-1H-pyrrolo[3,2-b]pyridine

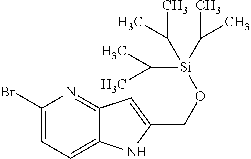 [SMILES: CC(C)[Si](OCc1cc2nc(Br)ccc2[nH]1)(C(C)C)C(C)C]

Chlorotriisopropylsilane (1.414 ml, 6.61 mmol) was added slowly to a solution of (5-bromo-1H-pyrrolo[3,2-b]pyridin-2-yl)methanol (1.00 g, 4.40 mmol) and DIPEA (0.769 ml, 4.40 mmol) in N,N-Dimethylformamide (DMF) (15 ml) at room temperature under nitrogen. The reaction was heated to 60° C. for 16 h. chlorotriisopropylsilane (0.471 ml, 2.202 mmol) and DIPEA (0.385 ml, 2.202 mmol) were added the reaction was heated at 70 deg C. for a further 7 h.

Saturated ammonium chloride (20 mL), water (20 mL) and EtOAc (80 mL) were added to the mixture, the organic phase was separated. The organic layer was then washed with water (3×40 mL), and dried over MgSO4. The volatiles were removed under reduced pressure to give a residue that was purified by normal phase chromatography, eluting 0-30% ethyl acetate in cyclohexane on a 80 g silica column to give 5-bromo-2-(((triisopropylsilyl)oxy)methyl)-1H-pyrrolo[3,2-b]pyridine (1.454 g, 3.79 mmol, 86% yield).

LCMS (System A, UV, ESI): Rₜ=1.60 min, [M+H]⁺ 383.2, 385.2

### Alternatively: Intermediate 7 5-bromo-2-({[tris(propan-2-yl)silyl]oxy}methyl)-1H-pyrrolo[3,2-b]pyridine

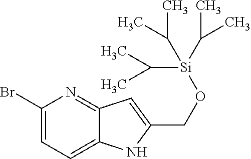 [SMILES: CC(C)[Si](OCc1cc2nc(Br)ccc2[nH]1)(C(C)C)C(C)C]

Chlorotriisopropylsilane (1.164 ml, 5.44 mmol) was added slowly to a solution of (5-bromo-1H-pyrrolo[3,2-b]pyridin-2-yl)methanol (1.029 g, 4.53 mmol) and triethylamine (0.821 ml, 5.89 mmol) in Dichloromethane (DCM) (30.9 ml) and N,N-Dimethylformamide (DMF) (10.30 ml) at room temperature under nitrogen. The reaction was heated to 60° C. for 60 hours triethylamine (0.189 ml, 1.360 mmol) and chlorotriisopropylsilane (0.291 ml, 1.360 mmol) was then added and the reaction was heated at 60° C. for a further 4 hours.

Saturated ammonium chloride (20 mL) and water (20 mL) were added to the mixture, the organic phase was separated. The organic layer was then washed with 5% aqueous lithium chloride solution (3×20 mL), dried by passing through hydrophobic frit and concentrated under reduced pressure. The resulting residue was purified by normal phase chromatography, eluting 0-30% ethyl acetate in cyclohexane on a 80 g silica column for 30 minutes to give 5-bromo-2-(((triisopropylsilyl)oxy)methyl)-1Hpyrrolo[3,2-b]pyridine (1.4457 g, 3.77 mmol, 83% yield).

LCMS (System A, UV, ESI): Rt=1.61 min, [M+H]+ 383.2, 385.2

### Intermediate 8 methyl 3-{[2-({[tris(propan-2-yl)silyl]oxy}methyl)-1H-pyrrolo[3,2-b]pyridin-5-yl]sulfonyl}propanoate

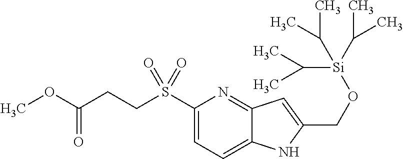 [SMILES: COC(=O)CCS(=O)(=O)c1ccc2[nH]c(CO[Si](C(C)C)(C(C)C)C(C)C)cc2n1]

A solution 5-bromo-2-(((triisopropylsilyl)oxy)methyl)-1H-pyrrolo[3,2-b]pyridine (5.000 g, 13.04 mmol), sodium 3-methoxy-3-oxopropane-1-sulfinate (3.41 g, 19.56 mmol) and copper(I) iodide (3.73 g, 19.56 mmol) in anhydrous Dimethyl Sulfoxide (DMSO) (30 ml) was placed under nitrogen and then heated to 110° C. for 1 h 20

The reaction mixture was cooled to RT and added to an aqueous ammonia solution (5%, 300 mL). The resulting mixture was extracted with EtOAc (100 mL). The organic phase was washed with water (50 mL), brine (100 mL) and dried over MgSO4. The volatiles were removed under reduced pressure to give a residue that was purified by column chromatography (Silica, 300 g column, wet load in DCM) using the elution gradient EtOAc in Cyclohexane 0-50% to give methyl 3-((2-(((triisopropylsilyl)oxy)methyl)-1H-pyrrolo[3,2-b]pyridin-5-yl)sulfonyl)propanoate (4.04 g, 8.89 mmol, 68.1% yield)

LCMS (System B, UV, ESI): Rₜ=1.43 min, [M+H]⁺ 455.2

### Intermediate 9 6-bromo-2-(prop-1-yn-1-yl)pyridin-3-amine

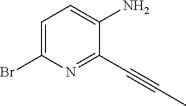

A mixture of 2,6-dibromopyridin-3-amine (50 g, 198 mmol), trimethyl(prop-1-yn-1-yl)silane (44.6 g, 397 mmol), bis(triphenylphosphine)palladium(II) chloride (13.93 g, 19.85 mmol), Et3N (83 mL, 595 mmol), TBAF (156 g, 595 mmol) and copper(I) iodide (11.34 g, 59.5 mmol) in Tetrahydrofuran (THF) (2000 mL) was stirred under nitrogen at room temperature for overnight.

The reaction mixture was evaporated and the residue partitioned between EtOAc (1000 mL) and water (1000 mL), extracted with EtOAc (500 mL*2). The organic phase was washed with saturated brine (500 mL), dried over sodium sulphate and evaporated in vacuo to give the crude product as a black oil.

The sample was preabsorbed on silica and purified on silica (Si) 660 g using a 0-50% ethyl acetate-petroleum ether gradient over 80 mins, flow rate 100 mL/min. The appropriate fractions were identified by UV absorbance (300 nm), combined and evaporated in vacuo to give the required product 6-bromo-2-(prop-1-yn-1-yl)pyridin-3-amine (16 g, 74.3 mmol, 37.4% yield), as a yellow solid.

1H NMR (400 MHz, DMSO-d6) δ ppm 7.20 (d, J=12 Hz, 1H) 7.00 (d, J=8 Hz, 1H) 5.78-5.67 (bs, 2H) 3.22-3.26 (m, 3H) 3.96 (br d, J=10.27 Hz, 1H) 4.14 (d, J=11.98 Hz, 1H) 2.11 (s, 3H)

LCMS (System C, UV, ESI): Rₜ=1.02 min, [M+H]⁺ 211,212

Similarly prepared were:

### Intermediate 10 6-bromo-2-(but-1-yn-1-yl)pyridin-3-amine

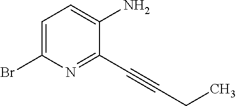

LCMS (System C, UV, ESI): Rₜ=0.95 min, [M+H]⁺ 225, 227

### Intermediate 11 6-chloro-2-((trimethylsilyl)ethynyl)pyridin-3-amine

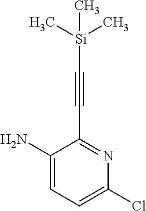

LCMS (System E, UV, ESI): Rₜ=2.57 min, [M+H]⁺ 225.19, 227.09

### Intermediate 12 5-bromo-2-methyl-1H-pyrrolo[3,2-b]pyridine

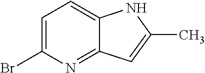

To a mixture of 6-bromo-2-(prop-1-yn-1-yl)pyridin-3-amine (15.00 g, 71.1 mmol) in N,N-Dimethylformamide (DMF) (400 mL) stirred under nitrogen at room temperature was added sodium hydride (5.69 g, 142 mmol). The reaction mixture was stirred at room temperature for overnight.

The reaction mixture was quenched with water, partitioned between EtOAc (1000 mL) and water (500 mL), extracted with EtOAc (500 mL*2). The organic phase was dried over sodium sulphate and evaporated in vacuo to give the crude product as a brown oil.

The sample was preabsorbed on silica and purified on silica (Si) 660 g using a 0-50% ethyl acetate-petroleum ether over 120 mins, flow rate 100 mL/min. The appropriate fractions were identified by UV absorbance (300 nm), combined and evaporated in vacuo to give the required product 5-bromo-2-methyl-1H-pyrrolo[3,2-b]pyridine (10.5204 g, 48.7 mmol, 68.5% yield), as a yellow solid.

1H NMR (400 MHz, DMSO-d6) δ 11.43 (s, 1H), 7.60 (dd, J=8.3, 0.9 Hz, 1H), 7.12 (d, J=8.3 Hz, 1H), 6.26 (dt, J=2.1, 1.0 Hz, 1H), 2.43 (s, 3H)

LCMS (System C, UV, ESI): Rₜ=1.15 min, [M+H]⁺ 211

Similarly prepared were:

### Intermediate 13 5-bromo-2-ethyl-1H-pyrrolo[3,2-b]pyridine

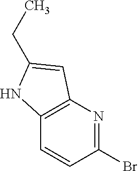

From Intermediate 10

1H NMR (400 MHz, DMSO-d6) δ 11.46 (s, 1H), 7.61 (dd, J=8.4, 0.8 Hz, 1H), 7.14 (d, J=8.4 Hz, 1H), 6.28 (dq, J=1.8, 0.8 Hz, 1H), 2.79 (qd, J=7.6, 0.8 Hz, 2H), 1.29 (t, J=7.6 Hz, 3H).

LCMS (System C, UV, ESI): Rₜ=0.95 min, [M+H]⁺ 225, 227

### Intermediate 14 5-chloro-1H-pyrrolo[3,2-b]pyridine

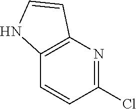

From Intermediate 11

LCMS (System E, UV, ESI): Rₜ=1.70 min, [M+H]⁺ 153.03, 154.94

### Intermediate 15 5-chloro-1-(phenylsulfonyl)-1H-pyrrolo[3,2-b]pyridine

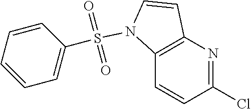 [SMILES: O=S(=O)(c1ccccc1)n1ccc2nc(Cl)ccc21]

To a stirred solution of 5-chloro-1H-pyrrolo[3,2-b]pyridine (20 g, 131 mmol) and DMAP (1.601 g, 13.11 mmol) in Dichloromethane (DCM) (200 mL) was added benzenesulfonyl chloride (21.97 mL, 170 mmol) and TEA (29.2 mL, 210 mmol). The reaction mixture was stirred under nitrogen at room temperature for 3 hours.

After completion of reaction, the reaction mixture was diluted with water (250 ml), extracted with DCM (300 ml×2). The combined organic layers were concentrated to afford 28 g brown gummy compound.

The crude product was pre-absorbed with silica gel (100-200 mesh), and purified by normal phase grace column chromatography through silica gel (100-200 mesh). Desired product was eluted with 30% EtOAc in pet ether, collected corresponding pure fractions was concentrated under vacuo to give as 5-chloro-1-(phenylsulfonyl)-1H-pyrrolo[3,2-b]pyridine (23.5 g, 80 mmol, 60.8% yield) as yellow solid.

LCMS (System E, UV, ESI): Rₜ=2.38 min, [M+H]⁺ 293.08.

Intermediate 16 5-chloro-2-methyl-1-(phenylsulfonyl)-1H-pyrrolo[3,2-b]pyridine

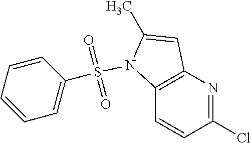 [SMILES: Cc1cc2nc(Cl)ccc2n1S(=O)(=O)c1ccccc1]

An oven-dried multi-necked flask was evacuated and purged with nitrogen (×3) and charged with Diisopropylamine (0.584 mL, 4.10 mmol) in anhydrous tetrahydrofuran (THF) (8.0 mL). The mixture was stirred and the flask placed in a cardice-acetone bath, prior to addition of n-butyl lithium (1.6 M in hexanes) (2.40 mL, 3.84 mmol). The flask was transferred to an ice-water bath and the mixture stirred for 0.5 h. The mixture was placed in a cardice-acetone bath prior to dropwise addition of 5-chloro-1-(phenylsulfonyl)-1H-pyrrolo[3,2-b]pyridine (750 mg, 2.56 mmol) in anhydrous tetrahydrofuran (4.8 mL). The mixture was stirred for 1 h. Methyl Iodide (0.320 mL, 5.12 mmol) was added and the reaction mixture was stirred and allowed to warm to RT.

Satd. aqueous ammonium chloride solution (10 mL) was added. The mixture was extracted with TBME (2×40 mL) and the combined organic phase was washed with water (20 mL), passed through a hydrophobic frit and evaporated in vacuo to afford an orange oil.

The oil was dissolved in DCM (2 mL), applied to a 120 g RediSep SiO2 column and eluted with a 0-40% gradient of EtOAc in cyclohexane, over 14 CV. The product containing fractions were combined and evaporated in vacuo to afford 5-chloro-2-methyl-1-(phenylsulfonyl)-1H-pyrrolo[3,2-b]pyridine (662 mg, 2.158 mmol, 84% yield), as a yellow oil.

LCMS (System A, UV, ESI): Rₜ=1.23 min, [M+H]⁺ 307.1.

### Intermediate 17 methyl 3-((2-methyl-1H-pyrrolo[3,2-b]pyridin-5-yl)sulfonyl)propanoate

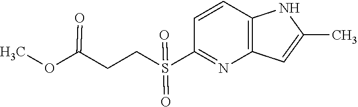

Sodium 3-methoxy-3-oxopropane-1-sulfinate (4.95 g, 28.4 mmol), copper(I) iodide (5.41 g, 28.4 mmol), 5-bromo-2-methyl-1H-pyrrolo[3,2-b]pyridine (4 g, 18.95 mmol) were dissolved in anhydrous Dimethyl Sulfoxide (DMSO) (46 mL) in a 20 mL microwave vial. The vial was sealed and the resulting mixture was degassed under a flow of nitrogen for 15 min, and heated to 110° C. for 3 h.

The mixture was diluted with EtOAc (50 mL) and the resulting mixture washed with Sat. aq. ammonium chloride (50 mL). The aq. contained desired product and was extracted with EtOAc (2×50 mL). The combined organic layers were then washed with sat. aq. sodium bicarbonate (50 mL). A large amount of solid was present, this was attempted to be dissolved in water and ethyl acetate—these layers were separated, the combined organics dried through a hydrophobic frit, concentrated in vacuo to give methyl 3-((2-methyl-1H-pyrrolo[3,2-b]pyridin-5-yl)sulfonyl)propanoate (3.616 g, 10.25 mmol, 54.1% yield) as an orange solid.

Due to low recovery and possible poor solubility of the product, the aqueous was extracted with ethyl acetate (2×20 mL). Solid remained in the hydrophobic frit and in the separating funnel—this was attempted to be dissolved in methanol and DCM. All organics were combined, concentrated in vacuo. The residue was partitioned between water (50 mL) and ethyl acetate (50 mL), aqueous extracted with ethyl acetate (3×50 mL) and the combined organics washed with water (50 mL), water and sodium bicarb (25 mL of each), sodium bicarb (2×20 mL), dried through a hydrophobic frit and concentrated in vacuo to give methyl 3-((2-methyl-1H-pyrrolo[3,2-b]pyridin-5-yl)sulfonyl)propanoate (1.799 g, 6.05 mmol, 31.9% yield) as a yellow/brown solid. 1H NMR (400 MHz, CHLOROFORM-d) δ ppm 2.56 (s, 3H) 2.79-2.87 (m, 2H) 3.66 (s, 3H) 3.71-3.77 (m, 2H) 6.53 (s, 1H) 7.66 (d, J=8.56 Hz, 1H) 7.79 (d, J=8.31 Hz, 1H) 8.59 (br s, 1H).

LCMS (System B, UV, ESI): Rₜ=0.66 min, [M+H]⁺ 282.97.

Similarly prepared was:

### Intermediate 18 methyl 3-({2-ethyl-1H-pyrrolo[3,2-b]pyridin-5-yl}sulfonyl) propanoate

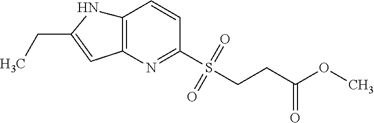 [SMILES: CCc1cc2nc(S(=O)(=O)CCC(=O)OC)ccc2[nH]1]

From Intermediate 13

1H NMR (400 MHz, DMSO-d6) δ ppm 1.32 (br t, J=7.58 Hz, 3H) 2.67 (br t, J=6.85 Hz, 2H) 2.85 (s, 2H) 3.52 (s, 3H) 3.65 (br t, J=6.72 Hz, 2H) 6.50 (s, 1H) 7.69 (br d, J=8.31 Hz, 1H) 7.90 (br d, J=8.31 Hz, 1H) 11.80 (br s, 1H).

LCMS (System B, UV, ESI): Rₜ=0.77 min, [M+H]⁺ 297.0.

### Intermediate 19 sodium 2-({[(tert-butoxy)carbonyl](methyl)amino}methyl)-1H-pyrrolo[3,2-b]pyridine-5-sulfinate

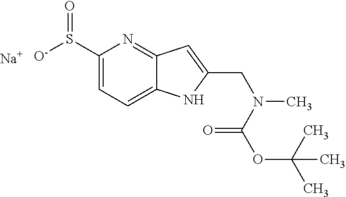 [SMILES: [Na+] ; CN(Cc1cc2nc(S(=O)[O-])ccc2[nH]1)C(=O)OC(C)(C)C]

Sodium methoxide (1.786 ml, 0.893 mmol) was added dropwise to tert-butyl ((5-((4-methoxy-3-oxobutyl)sulfonyl)-1H-pyrrolo[3,2-b]pyridin-2-yl)methyl)(methyl)carbamate (380 mg, 0.893 mmol) in Tetrahydrofuran (THF) (10 ml). The reaction mixture was stirred at 21° C. for 30 min.

The reaction mixture was concentrated under reduced pressure to give 2-(((tert-butoxycarbonyl)(methyl)amino)methyl)-1H-pyrrolo[3,2-b]pyridine-5-sulfinate, Sodium salt (310 mg, 0.892 mmol, 100% yield) as a white solid.

LCMS (System B, UV, ESI): Rₜ=0.61 min, [M+H]⁺ 325.

Similarly prepared were:

### Intermediate 20 2-({[tris(propan-2-yl)silyl]oxy}methyl)-1H-pyrrolo[3,2-b]pyridine-5-sulfinate sodium salt

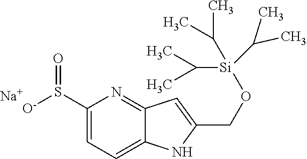 [SMILES: [Na+] ; CC(C)[Si](OCc1cc2nc(S(=O)[O-])ccc2[nH]1)(C(C)C)C(C)C]

From Intermediate 8

LCMS (SystemA, UV, ESI): Rₜ=0.96 min, [M+H]⁺ 369.3.

### Intermediate 21 2-methyl-1H-pyrrolo[3,2-b]pyridine-5-sulfinate, Sodium salt

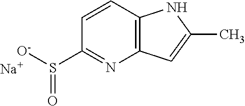

From Intermediate 17

1H NMR (400 MHz, DMSO-d6) δ ppm 2.41 (s, 3H) 6.24 (s, 1H) 7.37 (d, J=8.30 Hz, 1H) 7.58 (d, J=8.31 Hz, 1H) 11.15 (br s, 1H).

LCMS (System B, UV, ESI): Rₜ=0.35 min, [M+H]⁺ 196.9.

### Intermediate 22 sodium 2-ethyl-1H-pyrrolo[3,2-b]pyridine-5-sulfinate

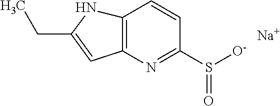

From Intermediate 18

1H NMR (400 MHz, DMSO-d6) δ ppm 1.29 (br t, J=7.46 Hz, 3H) 2.77 (q, J=7.34 Hz, 2H) 6.23 (br s, 1H) 7.38 (br d, J=7.83 Hz, 1H) 7.58 (br d, J=8.07 Hz, 1H) 11.05 (br s, 1H).

LCMS (System B, UV, ESI): Rₜ=0.77 min, [M+H]⁺ 211.0.

### Intermediate 23 2-chloro-N-[(2S)-1-hydroxybutan-2-yl]acetamide

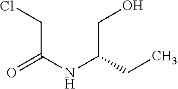

To a stirred solution of (S)-2-aminobutan-1-ol (673 mg, 7.55 mmol) and triethylamine (1.4 mL, 10.04 mmol) in anhydrous Dichloromethane (DCM) (16 mL) at −78° C. was added 2-chloroacetyl chloride (0.6 mL, 7.53 mmol) (diluted in 5.4 mL anhydrous DCM) dropwise over the period of 40 minutes with a syringe pump. The reaction mixture was quenched with 25 mL 0.5 M NaOH aqueous solution and warmed to RT over an hour. The mixture was diluted in ethyl acetate (30 mL), acidified with 2M aqueous HCl solution (approx 30 mL) until pH=approx 5 and transferred to a separating funnel. The organic layer was separated and the aqueous layer was back-extracted with ethyl acetate (20 mL). Organic layers were combined, dried over a hydrophobic frit and concentrated under reduced pressure.

The crude product was dissolved in ethyl acetate (10 mL), washed with brine (10 mL), dried over a hydrophobic frit and concentrated under reduced pressure to reveal 2-chloro-N-[(2S)-1-hydroxypropan-2-yl]acetamide (644 mg, 44%).

LCMS (System B, UV, ESI): Rt=0.44 min, [M+H+] 166.1.

### Intermediate 24 (S)-5-ethylmorpholin-3-one

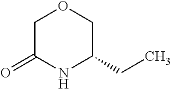

Sodium tert-butoxide solution, 2M in THF (4.6 mL, 9.20 mmol) was added dropwise to a stirred solution of (S)-2-chloro-N-(1-hydroxybutan-2-yl)acetamide (644 mg, 3.29 mmol) in Tetrahydrofuran (THF) (5 mL) at 0° C. over the period of 15 minutes.

Reaction mixture was warmed to RT and neutralised with 2M HCl aq. solution until pH=approx 7. The mixture was diluted in DCM (30 mL) and washed with water (20 mL). The organic layer was separated and the aqueous layer was back-extracted with 20 mL DCM. The organic layers were combined, dried over a hydrophobic frit and concentrated under reduced pressure.

The solid was dissolved in 1 mL DCM and purified using normal phase chromatography, eluting with 35-60% 3:1 ethyl acetate:ethanol+1% NEt₃ in cyclohexane through a 24 g silica column at a flow rate of 32 ml/min over 20 CV's. The desired fractions combined and concentrated under high vacuum to afford (S)-5-ethylmorpholin-3-one as a white solid, 220 mg.

LCMS (System B, UV, ESI): Rt=0.44 min, [M+H+] 130.1.

### Intermediate 25 methyl (S)-2-chloro-6-(3-ethylmorpholino)pyrimidine-4-carboxylate

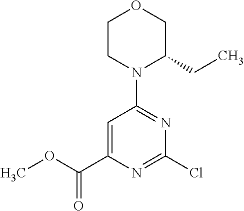 [SMILES: CC[C@H]1COCCN1c1cc(C(=O)OC)nc(Cl)n1]

To methyl 2,6-dichloropyrimidine-4-carboxylate (20 g, 97 mmol) and (S)-3-ethylmorpholine, Hydrochloride (14.65 g, 97 mmol) in Dimethyl Sulfoxide (DMSO) (40 mL) was added triethylamine (40.4 mL, 290 mmol) and the reaction was stirred at room temperature for 1 hour.

The reaction mixture was partitioned between ethyl acetate (300 mL) and water (300 mL), and extracted with ethyl acetate (150 mL×3). The organic phase was dried over sodium sulphate and evaporated in vacuo to give the crude product methyl (S)-2-chloro-6-(3-ethylmorpholino)pyrimidine-4-carboxylate (25 g, 78 mmol, 81% yield) as a yellow oil.

LCMS (System A, UV, ESI): Rₜ=1.03 min, [M+H]⁺ 286.

### Intermediate 26 (S)-2-bromo-6-(3-ethylmorpholino)isonicotinic acid

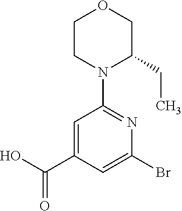 [SMILES: CC[C@H]1COCCN1c1cc(C(=O)O)cc(Br)n1]

2,2,6,6-Tetramethylpiperidine (24.82 ml, 147 mmol) was added to 2,6-di bromoisonicotinic acid (4.9188 g, 17.51 mmol) and (S)-3-ethylmorpholine hydrochloride (3.19 g, 21.01 mmol). The vials containing reaction mixture were sealed, heated to 200° C. and stirred at 200° C. for 36.75 hr.

The reaction mixture was partitioned between 300 mL Ethyl acetate and 300 mL water acidified with 2M HCl (pH ˜0-1). Organic layer removed, aqueous layer back-extracted with 2×200 mL DCM. The organic layers were combined, dried over a hydrophobic frit, and concentrated under reduced pressure. TMP was then azeotroped with 20 mL water and removed under reduced pressure.

Residue was treated with 20 mL DMSO, and stirred at 70 degrees which formed an off white solid and a brown supernatant. Mixture was then cooled down in the hot plate slowly. The mixture was filtered on hydrophobic frit under vacuum and washed with TBME then concentrated under reduced pressure to give (S)-2-bromo-6-(3-ethylmorpholino)isonicotinic acid (3.774 g, 11.97 mmol, 68.4% yield) as a beige solid.

LCMS (System B, UV, ESI): Rₜ=1.15 min, [M+H]⁺ 317.0.

### Intermediate 27 (S)-(2-chloro-6-(3-ethylmorpholino)pyrimidin-4-yl)methanol

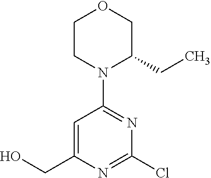 [SMILES: CC[C@H]1COCCN1c1cc(CO)nc(Cl)n1]

Methyl (S)-2-chloro-6-(3-ethylmorpholino)pyrimidine-4-carboxylate (24 g, 84 mmol) in Methanol (200 mL) was added sodium tetrahydroborate (6.36 g, 168 mmol) at 0° C. The reaction was stirred at room temperature for 1 hour.

The reaction mixture was quenched with water (200 mL), partitioned between ethyl acetate (300 mL) and water (300 mL), and extracted with ethyl acetate (150 mL×3). The organic phase was dried over sodium sulphate and evaporated in vacuo to give the crude product methyl (S)-2-chloro-6-(3-ethyl-morpholino)pyrimidine-4-carboxylate (25 g, 87 mmol, 91% yield) as yellow oil.

The sample was preabsorbed on silica and purified on a silica (Si) 660 g using a 0%-30% ethyl acetate-petroleum solvent gradient over 80 mins, Flow rate: 100 mL/min. The appropriate fractions were identified by UV absorbance (254 nm), combined and evaporated in vacuo to give the desired product (S)-(2-chloro-6-(3-ethylmorpholino)pyrimidin-4-yl)methanol (14.3472 g, 55.5 mmol, 66.1% yield) as a light yellow solid.

1H-NMR (400 MHz, DMSO-d6): δ [ppm] 6.74 (s, 1H), 5.54-5.51 (t, 1H), 4.33-4.29 (m, 2H), 4.24-3.90 (m, 2H), 3.89-3.80 (m, 2H), 3.52-3.38 (m, 2H), 3.19-3.13 (m, 1H), 1.80-1.63 (m, 2H), 0.93-0.81 (m, 3H).

LCMS (System A, UV, ESI): Rₜ=1.97 min, [M+H]⁺ 258.

### Intermediate 28 (S)-(2-bromo-6-(3-ethylmorpholino)pyridin-4-yl)methanol

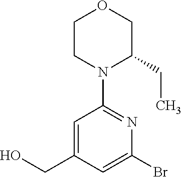 [SMILES: CC[C@H]1COCCN1c1cc(CO)cc(Br)n1]

BH3.THF (14.02 ml, 14.02 mmol) was added to (S)-2-bromo-6-(3-ethylmorpholino) isonicotinic acid (4.4179 g, 14.02 mmol) in dry Tetrahydrofuran (THF) (75 ml) at 0° C. The reaction mixture was stirred at 0° C. to RT for 17 hr.

The reaction mixture was then cooled to 0° C. and quenched by dropwise addition of MeOH (5 mL)—effervescence was observed. The mixture was stirred for 45 mins at 0° C. After this period further addition of 2M HCl (a few drops) produced no supplementary effervescence. The reaction mixture was then concentrated under reduced pressure. The residue was partitioned between 125 mL EtOAc and 125 mL saturated aqueous NH₄Cl solution. The organic layer was taken, dried over a hydrophobic frit, and concentrated under reduced pressure to give (S)-(2-bromo-6-(3-ethylmorpholino)pyridin-4-yl)methanol as a yellow gum.

LCMS (System B, UV, ESI): Rₜ=0.98 min, [M+H]⁺ 301.1, 303.1.

Similarly prepared was:

### Intermediate 29 (2,6-dibromopyridin-4-yl)methanol

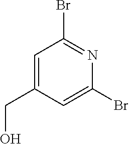

From 2,6-dibromoisonicotinic acid

LCMS (System B, UV, ESI): Rₜ=0.78 min, [M+H]⁺ 268

### Intermediate 30 2,6-dibromo-4-((methylsulfonyl)methyl)pyridine

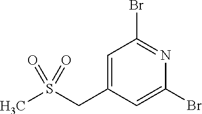

Triethylamine (2.190 mL, 15.71 mmol) and mesyl-Cl (0.898 mL, 11.52 mmol) were added to (2,6-dibromopyridin-4-yl)methanol (2796 mg, 10.48 mmol) in dry Acetonitrile (50 mL) under nitrogen at 0° C. The reaction mixture was stirred at 0° C. for 60 min, warmed to RT and stirred for 2 h.

Mesyl-Cl (0.18 mL, 2.310 mmol) was added and the resulting mixture was stirred at RT for 5 min.

Sodium methanesulfinate (2139 mg, 20.95 mmol) and potassium iodide (522 mg, 3.14 mmol) were added and the volatiles were removed under reduced pressure. Acetonitrile (50 mL) was added and the resulting mixture heated to reflux for 17 h.

The reaction mixture was concentrated under reduced pressure. The residue was partitioned between ethyl acetate (100 ml) and saturated aqueous ammonium chloride (100 ml). The organic phases were washed with water (75 ml) and brine (75 ml), dried over magnesium sulfate and concentrated under reduced pressure to give a solid residue (3.264 g). The crude product was recrystallised from ethanol (approx 50 ml) to give 2,6-dibromo-4-((methylsulfonyl)methyl)pyridine (2.144 g, 6.52 mmol, 62.2% yield) as an off-white solid.

The mother liquor was dried over reduced pressure to give a residue that was triturated in Ethanol (20 mL), filtered and washed with additional Ethanol (10 mL) to give 2,6-dibromo-4-((methylsulfonyl)methyl)pyridine (267 mg, 0.812 mmol, 7.75% yield) as an off-white solid.

LCMS (System B, UV, ESI): Rₜ=0.95 min, [M+H]⁺ 327.9.

### Intermediate 31 6-((methylsulfonyl)methyl)pyrimidine-2,4(1H,3H)-dione

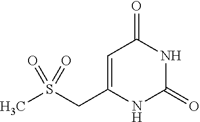

A solution of 6-(chloromethyl)pyrimidine-2,4(1H,3H)-dione (1 g, 6.23 mmol), sodium methanesulfinate (0.827 g, 8.10 mmol) and potassium iodide (0.207 g, 1.246 mmol) in dry Acetonitrile (25 ml) under an atmosphere of nitrogen was heated to reflux for 3 h.

The mixture was cooled down and the volatiles were removed under reduced pressure to give a solid residue that was triturated in water (25 mL), filtered. The residual solid was washed with water (10 mL), dried under aspiration and in high vacuum overnight to give 6-((methylsulfonyl)methyl)pyrimidine-2,4(1H,3H)-dione (998 mg, 4.64 mmol, 74.5% yield) as a colourless solid.

¹H NMR (400 MHz, DMSO-d6) δ 11.14 (1H, br. s., NH), 10.93 (1H, br. s., NH), 5.61 (1H, s, C—H), 4.25 (2H, s, CH₂), 2.99-3.17 (3H, m, Me).

LCMS (SystemB, UV, ESI): Rₜ=0.20 min, [M+H]⁺ 205.

### Intermediate 32 2,4-dichloro-6-(methanesulfonylmethyl)pyrimidine

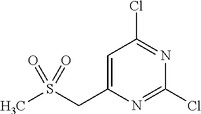

6-((Methylsulfonyl)methyl)pyrimidine-2,4(1H,3H)-dione (3.90 g, 19.10 mmol) was suspended in POCl3 (35 ml, 376 mmol) under an atmosphere of nitrogen and the resulting mixture was stirred at reflux for 5 h.

The mixture was cooled down and toluene (100 mL) was added. The volatiles were removed under reduced pressure and the solid residue was taken in DCM (80 mL), filtered, and washed with DCM (20 mL). The solid was dried under vacuum to give 2,4-dichloro-6-((methylsulfonyl)methyl)pyrimidine (3.30 g, 13.00 mmol, 68.1% yield) as a grey solid. The filtrate was added to a vigorously stirred saturated sodium hydrogen carbonate solution (100 mL) to quench residual POCl3. The resulting mixture was stirred for 10 min at room temperature and the phases were separated. The volatiles were removed under reduced pressure to give as an off-white/brown sticky solid, which was triturated in Et2O (5 mL) and filtered. The residual solid was washed with Et2O (5 mL), dried under vacuum to give 2,4-dichloro-6-((methylsulfonyl)methyl)pyrimidine (824 mg, 3.42 mmol, 17.90% yield) as an off-white solid.

LCMS (SystemB, UV, ESI): Rₜ=0.60 min, [M+H]⁺ 240.9

### Intermediate 33 2,6-dichloro-4-((methylsulfonyl)methyl)pyridine

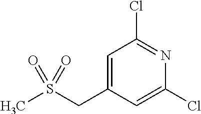

A solution of 2,6-dichloro-4-(chloromethyl)pyridine (5 g, 25.5 mmol), sodium methanesulfinate (3.90 g, 38.2 mmol) and potassium iodide (0.845 g, 5.09 mmol) in Acetonitrile (100 mL) (anhydrous) was refluxed for 1 h 30 mins.

The reaction mixture was cooled to RT and the volatiles were removed under reduced pressure. The solid residue was mixture was partitioned between water (60 mL) and EtOAc (120 mL). The phases were separated and the organic phase was washed with water (60 mL), brine (40 mL) and dried over MgSO4. The volatiles were removed under reduced pressure to give 2,6-dichloro-4-((methylsulfonyl)methyl)pyridine (5.2 g, 21.66 mmol, 85% yield) as an off-white solid.

LCMS (SystemB, UV, ESI): Rₜ=0.73 min, [M+H]⁺ 240.0

### Intermediate 34 (3S)-4-[6-(bromomethyl)-2-chloropyrimidin-4-yl]-3-ethylmorpholine

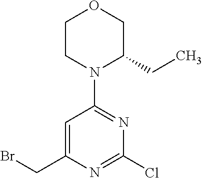 [SMILES: CC[C@H]1COCCN1c1cc(CBr)nc(Cl)n1]

1-bromopyrrolidine-2,5-dione (1850 mg, 10.39 mmol) was added portionwise to a solution of (S)-(2-chloro-6-(3-ethylmorpholino)pyrimidin-4-yl)methanol (2010 mg, 7.80 mmol), triphenylphosphine (2660 mg, 10.14 mmol) in 30 mL THF, and the mixture was stirred at RT for 60 min.

The reaction mixture was diluted with 70 mL saturated sodium bicarbonate solution and 70 mL ethyl acetate. The organic layer was taken, dried over a hydrophobic frit, and concentrated under reduced pressure. The residue was stood at rt for 16 h. The residue (˜5.5 mL of oil) was diluted with 0.5 mL DCM, and eluted on a 120 g silica gel column in cyclohexane with a gradient of 0-100% EtOAc over 16 cv. The desired fractions were concentrated under reduced pressure to give (S)-4-(6-(bromomethyl)-2-chloropyrimidin-4-yl)-3-ethylmorpholine (2458 mg, 7.67 mmol, 98% yield) as an orange oil.

LCMS (System A, UV, ESI): Rₜ=1.07 min, [M+H]⁺ 319.9

Similarly prepared was:

### Intermediate 35 (S)-4-(6-bromo-4-(bromomethyl)pyridin-2-yl)-3-ethylmorpholine

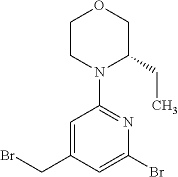 [SMILES: CC[C@H]1COCCN1c1cc(CBr)cc(Br)n1]

To a solution of (S)-(2-bromo-6-(3-ethylmorpholino)pyridin-4-yl)methanol (4.22 g, 14.01 mmol) in Tetrahydrofuran (THF) (90 mL), triphenylphosphine (4.54 g, 17.31 mmol) then 1-bromopyrrolidine-2,5-dione (2.98 g, 16.74 mmol) were added at room temperature, and the mixture was stirred at RT for 30 min. Then diluted with 100 mL saturated sodium bicarbonate solution and 100 mL ethyl acetate. The layers were partitioned. The organic layer was taken, dried over a hydrophobic frit, and concentrated under reduced pressure. The residue was dissolved in 10 mL DCM, and eluted on a 120 g silica gel column in cyclohexane with a gradient of 0-25% EtOAc over 12CV. The desired fractions were concentrated under reduced pressure to give (S)-4-(6-bromo-4-(bromomethyl)pyridin-2-yl)-3-ethylmorpholine (502 mg, 1.379 mmol, 9.84% yield) as a colourless oil.

LCMS (System A, UV, ESI): Rₜ=1.36 min, [M+H]⁺ 363.0, 365.0, 367.0

### Intermediate 36 (3S)-4-[2-chloro-6-(methanesulfonylmethyl)pyrimidin-4-yl]-3-ethylmorpholine

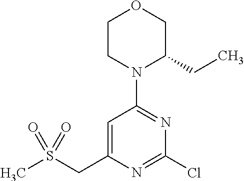 [SMILES: CC[C@H]1COCCN1c1cc(CS(C)(=O)=O)nc(Cl)n1]

To a solution of (S)-4-(6-(bromomethyl)-2-chloropyrimidin-4-yl)-3-ethylmorpholine (1242 mg, 3.87 mmol) in Acetonitrile (14 mL), 1-bromopyrrolidine-2,5-dione (1042 mg, 5.85 mmol) was added at room temperature, and the mixture was stirred at 40° C. for 1.5 h. sodium methanesulfinate (646 mg, 6.33 mmol) was then added and the reaction mixture was refluxed for 2.5 h. This was then cooled to rt and stood for 14 h. sodium methanesulfinate (428 mg, 4.19 mmol) and potassium iodide (202 mg, 1.217 mmol) were then added and the reaction mixture was refluxed for 1 h.

The reaction mixture was diluted with 100 mL water and 100 mL EtOAc—salt was added to separate the layers. The organic layer was removed and the aqueous layer was back-extracted with 100 mL EtOAc. The organic layers were combined, dried over a hydrophobic frit, and concentrated under reduced pressure. The residue was dissolved in 5 mL DCM and eluted on a silica gel column in cyclohexane with a gradient of 0-100% EtOAc. The desired fractions were concentrated under reduced pressure to give (2-chloro-6-((methylsulfonyl)methyl)pyrimidin-4-yl)-3-ethylmorpholine contaminated with ˜2 eq succinimide and so was dissolved in 2.5 mL DMSO and eluted on an xbridge column in 10 mM ammonium bicarbonate solution with a gradient of 5-95% acetonitrile. Collected fractions were concentrated under reduced pressure to give (S)-4-(2-chloro-6-((methylsulfonyl)methyl)pyrimidin-4-yl)-3-ethylmorpholine (168 mg, 0.525 mmol, 13.56% yield) as a white solid.

LCMS (SystemB, UV, ESI): Rₜ=0.81 min, [M+H]⁺ 320.2

Alternatively:

### Intermediate 36 (3S)-4-[2-chloro-6-(methanesulfonylmethyl)pyrimidin-4-yl]-3-ethylmorpholine

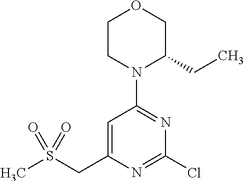 [SMILES: CC[C@H]1COCCN1c1cc(CS(C)(=O)=O)nc(Cl)n1]

DIPEA (950 μl, 5.44 mmol) was added to 2,4-dichloro-6-((methylsulfonyl)methyl)pyrimidine (503.1 mg, 2.087 mmol) and (S)-3-ethylmorpholine hydrochloride (379.3 mg, 2.501 mmol) in Dimethyl Sulfoxide (DMSO) (7000 μl). The reaction mixture was sealed and stirred at room temperature for 4.25 h.

The reaction mixture was partitioned between ethyl acetate (80 ml) and saturated ammonium chloride (40 ml). The aqueous phase was further extracted with ethyl acetate (2×40 ml). The organic phases were combined, washed with water (40 ml) and brine (40 ml), dried over magnesium sulfate and concentrated under reduced pressure. The crude product was adsorbed on florisil and the volatiles were removed under vacuum. The crude product adsorbed on solid phase (florisil) was purified by column chromatography on silica (80 g) using the elution gradient ethyl acetate in cyclohexane 20 to 100% to yield (S)-4-(4-chloro-6-((methylsulfonyl)methyl) pyrimidin-2-yl)-3-ethylmorpholine (42.3 mg, 0.132 mmol, 6.34% yield).

LCMS (System B, UV, ESI): Rₜ=0.81 min, [M+H]⁺ 320.1

Similarly prepared were:

### Intermediate 37 (S)-4-(6-bromo-4-((methylsulfonyl)methyl)pyridin-2-yl)-3-ethylmorpholine

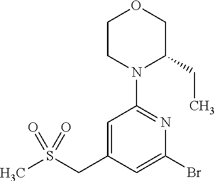 [SMILES: CC[C@H]1COCCN1c1cc(CS(C)(=O)=O)cc(Br)n1]

From Intermediate 30

LCMS (System B, UV, ESI): Rₜ=0.99 min, [M+H]⁺ 365.1

### Intermediate 38 (S)-4-(6-chloro-4-((methylsulfonyl)methyl)pyridin-2-yl)-3-ethylmorpholine

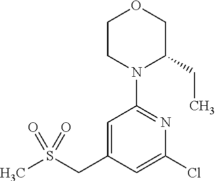 [SMILES: CC[C@H]1COCCN1c1cc(CS(C)(=O)=O)cc(Cl)n1]

From Intermediate 33

LCMS (System A, UV, ESI): Rₜ=1.00 min, [M+H]⁺ 319.1

### Intermediate 39 (3S)-4-[2-chloro-6-(4-methanesulfonyloxan-4-yl)pyrimidin-4-yl]-3-ethylmorpholine

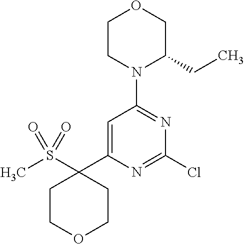 [SMILES: CC[C@H]1COCCN1c1cc(C2(S(C)(=O)=O)CCOCC2)nc(Cl)n1]

1-bromo-2-(2-bromoethoxy)ethane (0.120 ml, 0.955 mmol), sodium hydroxide (283 mg, 7.08 mmol) and tetrabutylammonium bromide (40.9 mg, 0.127 mmol) were added to (S)-4-(2-chloro-6-((methylsulfonyl)-methyl)pyrimidin-4-yl)-3-ethylmorpholine (200 mg, 0.625 mmol) in Toluene (12.508 ml). The reaction was sealed, heated to 90° C. and stirred at 90° C. for 2.25 h.

The reaction mixture was concentrated under reduced pressure. The residue was partitioned between ethyl acetate (50 ml) and water (40 ml). The aqueous phase was further extracted with ethyl acetate (25 ml), the organic phases combined, washed with brine (40 ml), dried over magnesium sulfate and concentrated under reduced pressure to yield (S)-4-(2-chloro-6-(4-(methylsulfonyl)tetrahydro-2H-pyran-4-yl)pyrimidin-4-yl)-3-ethylmorpholine (306.2 mg, 0.628 mmol, 100% yield).

LCMS (System B, UV, ESI): Rₜ=0.97 min, [M+H]⁺ 390.2.

Similarly prepared was:

### Intermediate 40 (S)-4-(6-chloro-4-(4-(methylsulfonyl)tetrahydro-2H-pyran-4-yl)pyridin-2-yl)-3-ethylmorpholine

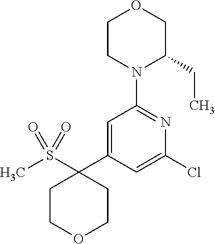 [SMILES: CC[C@H]1COCCN1c1cc(C2(S(C)(=O)=O)CCOCC2)cc(Cl)n1]

From Intermediate 38

LCMS (System A, UV, ESI): Rₜ=1.04 min, [M+H]⁺ 389.2

### Intermediate 41 tert-butyl 4-{2-chloro-6-[(3S)-3-ethylmorpholin-4-yl]pyrimidin-4-yl}-4-methanesulfonylpiperidine-1-carboxylate

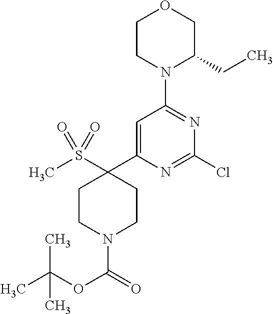 [SMILES: CC[C@H]1COCCN1c1cc(C2(S(C)(=O)=O)CCN(C(=O)OC(C)(C)C)CC2)nc(Cl)n1]

(S)-4-(2-Chloro-6-((methylsulfonyl)methyl)pyrimidin-4-yl)-3-ethylmorpholine (300 mg, 0.938 mmol), TBAI (69.3 mg, 0.188 mmol) and tert-butyl bis(2-chloroethyl)carbamate (310 μl, 1.407 mmol) were placed in dry N,N-Dimethylformamide (DMF) (7000 μl) under an atmosphere of nitrogen. The resulting mixture was stirred for 5 min at RT, then cooled to 0° C. (ice bath). sodium hydride (113 mg, 2.81 mmol) was added and the resulting mixture was stirred in a melting ice bath for 18 h (0° C. to RT). The reaction mixture was heated at 40° C. for 24 h. Further sodium hydride (113 mg, 2.81 mmol) was added and the reaction mixture heated at 60° C. for 24 h. The reaction mixture was quenched by careful addition of ammonium chloride saturated solution (10 mL) at 0° C. The resulting mixture was extracted with EtOAc (50 mL). The organic phase was washed with water (80 mL), brine (40 mL) and dried over MgSO4. The volatiles were removed under reduced pressure and the residue purified by normal phase chromatography on silica (Si) 50 g and eluted using a 0-100% ethyl acetate-cyclohexane gradient over 40 mins. The appropriate fractions were combined and evaporated in vacuo to give the required product tert-butyl 4-{2-chloro-6-[(3S)-3-ethylmorpholin-4-yl]pyrimidin-4-yl}-4-methanesulfonylpiperidine-1-carboxylate as a yellow gum. containing significant impurities.

LCMS (System B, UV, ESI): Rt=1.29 min, [M+H+] 489

Similarly prepared was:

### Intermediate 42 tert-butyl (S)-4-(2-bromo-6-(3-ethylmorpholino)pyridin-4-yl)-4-(methylsulfonyl)piperidine-1-carboxylate

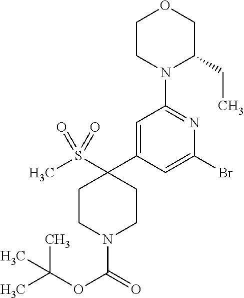 [SMILES: CC[C@H]1COCCN1c1cc(C2(S(C)(=O)=O)CCN(C(=O)OC(C)(C)C)CC2)cc(Br)n1]

Sodium hydride (80 mg, 2.000 mmol) was added to a solution of (S)-4-(6-bromo-4-((methylsulfonyl)methyl)pyridin-2-yl)-3-ethylmorpholine (270 mg, 0.743 mmol), TBAI (57 mg, 0.154 mmol) and tert-butyl bis(2-chloroethyl)carbamate (0.25 mL, 1.136 mmol) in dry N,N-Dimethylformamide (DMF) (5 mL) under an atmosphere of nitrogen at 0° C. (ice bath). The reaction was stirred in a melting ice bath for 16 h. The mixture was left to stir at room temperature under nitrogen for 3 days. tert-butyl bis(2-chloroethyl)carbamate (0.25 mL, 1.136 mmol) was added and the mixture was stirred at room temperature for 48 h. tert-butyl bis(2-chloroethyl) carbamate (0.1 mL, 0.454 mmol) was added and the mixture was left to stir at room temperature for 24 h.

The mixture was quenched by careful addition of sat. ammonium chloride solution (10 mL). The mixture was left to stir for 5 min. The mixture was then diluted with EtOAc (30 mL) and water (10 mL). The organic layer was washed with brine (20 mL). The organic was separated and passed through an hydrophobic frit. Solvent was removed under reduced pressure to give crude product.

The crude product was purified by reverse phase chromatography. The column used was 60 g Redisep C18 column. The compound was dissolved in a minimum of DMSO/MeOH and loaded onto the top of the column by injection after equilibration. The product was then eluted using 50-95% CH₃CN+0.1% formic acid (B)/H2O+0.1% formic acid (A) gradient. The fractions were collected by UV detection. A selection of fractions were concentrated under reduced pressure to give tert-butyl (S)-4-(2-bromo-6-(3-ethylmorpholino)pyridin-4-yl)-4-(methylsulfonyl)piperidine-1-carboxylate.

LCMS (System B, UV, ESI): Rₜ=1.27 min, [M+H]⁺ 532, 534

### Intermediate 43 (5S)-4-[2-chloro-6-(methanesulfonylmethyl)pyrimidin-4-yl]-5-ethylmorpholin-3-one

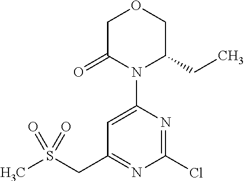 [SMILES: CC[C@H]1COCC(=O)N1c1cc(CS(C)(=O)=O)nc(Cl)n1]

2,4-dichloro-6-((methylsulfonyl)methyl)pyrimidine (180 mg, 0.747 mmol), (S)-5-ethylmorpholin-3-one (106 mg, 0.821 mmol), palladium(II) acetate (33.5 mg, 0.149 mmol), xantphos (173 mg, 0.299 mmol) and Cs2CO3 (365 mg, 1.120 mmol) were added to a microwave vial which was sealed and purged/filled with nitrogen/vacuum. Anhydrous 1,4-Dioxane (5 ml) was then added and the solution was sparged with nitrogen for 5 minutes. The reaction mixture was stirred at rt for 7 hr and stood overnight at room temperature.

Reaction mixture was diluted with DCM (10 mL), filtered through a celite cartridge (2.5 g) under nitrogen (washing with DCM (2×10 mL), and concentrated in vacuo to give 451 mg

The residue was taken in minimal DCM and purified by flash chromatography (silica, 40 g) eluting with 0-100% ethyl acetate in cyclohexane over 16 CV. Appropriate fractions were combined, concentrated in vacuo, taken in minimal DCM and concentrated under nitrogen to give (S)-4-(2-chloro-6-((methylsulfonyl)methyl)pyrimidin-4-yl)-5-ethylmorpholin-3-one (207 mg, 0.589 mmol, 79% yield) as an orange/yellow oily solid.

1H NMR (400 MHz, DMSO-d6) δ ppm 0.92 (t, J=7.46 Hz, 3H) 1.59-1.71 (m, 1H) 1.73-1.86 (m, 1H) 3.11 (s, 3H) 3.89 (dd, J=12.47, 1.71 Hz, 1H) 4.06 (d, J=12.72 Hz, 1H) 4.31 (d, J=17.36 Hz, 1H) 4.41 (d, J=17.61 Hz, 1H) 4.52 (br d, J=9.78 Hz, 1H) 4.78 (s, 2H) 8.43 (s, 1H)

LCMS (System B, UV, ESI): Rt=0.88 min, [M+H+] 333.9, 335.8

### Intermediate 44 (S)-2-((6-bromo-4-((methylsulfonyl)methyl)pyridin-2-ylamino)butan-1-ol

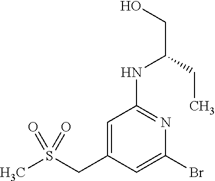 [SMILES: CC[C@@H](CO)Nc1cc(CS(C)(=O)=O)cc(Br)n1]

The mixture of 2,6-dibromo-4-((methylsulfonyl)methyl)pyridine (8.9 g, 27.1 mmol), (S)-2-((6-bromo-4-((methylsulfonyl)methyl)pyridin-2-yl)amino)butan-1-ol (7.5 g, 21.89 mmol, 81% yield) and 2,2,6,6-tetramethylpiperidine (65.0 g, 460 mmol) was stirred at 150° C. for overnight.

The reaction mixture was cooled to room temperature and partitioned between ethyl acetate (500 mL) and water (800 mL), and extracted with ethyl acetate (300 mL×3). The organic phase was dried over sodium sulphate and evaporated in vacuo to give the crude product as yellow oil.

The sample was preabsorbed on silica and purified on a silica (Si) 330 g using a 0%-50% ethyl acetate-petroleum solvent gradient over 120 mins, Flow rate: 70 mL/min. The appropriate fractions were combined and evaporated in vacuo to give desired product (S)-2-((6-bromo-4-((methylsulfonyl)methyl)pyridin-2-yl)amino)butan-1-ol (7.5 g, 21.89 mmol, 81% yield) as yellow oil.

LCMS (System C, UV, ESI): Rₜ=0.825 min, [M+H]⁺ 337.1

Similarly prepared was:

### Intermediate 92 (S)-2-((6-chloro-4-((methylsulfonyl)methyl)pyridin-2-yl)amino) butan-1-ol

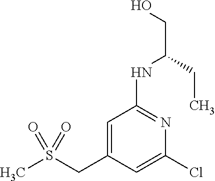 [SMILES: CC[C@@H](CO)Nc1cc(CS(C)(=O)=O)cc(Cl)n1]

From Intermediate 33

LCMS (System B, UV, ESI): Rₜ=0.72 min, [M+H]⁺ 337.1

### Intermediate 45 ethyl (S)-2-(2-((6-bromo-4-((methylsulfonyl)methyl)pyridin-2-yl)amino)butoxy)acetate

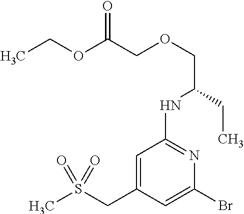 [SMILES: CCOC(=O)COC[C@H](CC)Nc1cc(CS(C)(=O)=O)cc(Br)n1]

To (S)-2-((6-bromo-4-((methylsulfonyl)methyl)pyridin-2-yl)amino)butan-1-ol (5.5 g, 16.31 mmol) and Rh(oct)4 (1.270 g, 1.631 mmol) in Dichloromethane (DCM) (100 mL) and stirred under nitrogen at room temp was added ethyl 2-diazoacetate (2.233 g, 19.57 mmol) in Dichloromethane (DCM) (3.0 mL) dropwise. The reaction mixture was stirred at 40° C. for 2 hours. Then ethyl 2-diazoacetate (1.11 g, 9.78 mmol) in dichloromethane (DCM) (2.0 mL) was added dropwise, after stirring 2 hours, ethyl 2-diazoacetate (1.11 g, 9.78 mmol) in dichloromethane (DCM) (2.0 mL) was added dropwise, and stirred for 2 hours. The reaction mixture was combined with a previous batch of 2 g (5.93 mmol), and partitioned between DCM 400 mL and water 400 mL, and extracted with DCM (300 mL×3). The organic phase was dried over sodium sulphate and evaporated in vacuo to give the crude product as yellow oil.

The sample was preabsorbed on silica and purified on a silica (Si) 330 g using a 0%-50% ethyl acetate-petroleum solvent gradient over 120 mins, Flow rate: 70 mL/min. The appropriate fractions were combined and evaporated in vacuo to give desired product ethyl (S)-2-(2-((6-bromo-4-((methylsulfonyl)methyl)pyridin-2-yl)amino)butoxy)acetate (5.3 g, 11.14 mmol, 50% yield) as yellow oil.

LCMS (System A, UV, ESI): Rₜ=1.11 min, [M+H]⁺ 423, 425

Similarly prepared was:

### Intermediate 93 ethyl (S)-2-(2-((6-chloro-4-((methylsulfonyl)methyl)pyridin-2-yl)amino)butoxy) acetate

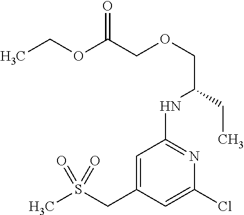 [SMILES: CCOC(=O)COC[C@H](CC)Nc1cc(CS(C)(=O)=O)cc(Cl)n1]

From Intermediate 92

LCMS (System B, UV, ESI): Rₜ=1.00 min, [M+H]⁺ 379.2

### Intermediate 46 (S)-2-(2-((6-bromo-4-((methylsulfonyl)methyl)pyridin-2-yl)amino)butoxy)acetic acid

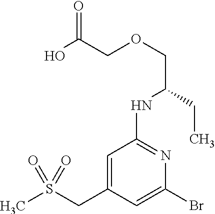 [SMILES: CC[C@@H](COCC(=O)O)Nc1cc(CS(C)(=O)=O)cc(Br)n1]

Ethyl (S)-2-(2-((6-bromo-4-((methylsulfonyl)methyl)pyridin-2-yl)amino) butoxy)acetate (4.3 g, 10.16 mmol) dissolved in Tetrahydrofuran (THF) (15 mL) and NaOH (15 ml, 30.0 mmol). The reaction mixture was stirred at room temperature for 2 hours.

The reaction mixture was combined with a previous batch of 1 g (2.36 mmol), and the combined reaction mixture was partitioned between (DCM:MeOH=10:1) 300 mL and water 300 mL, and extracted with (DCM:MeOH=10:1) (150 mL×5). The organic phase was dried over sodium sulphate and evaporated in vacuo to give (S)-2-(2-((6-bromo-4-((methylsulfonyl) methyl)pyridin-2-yl)amino)butoxy)acetic acid (4.6 g, 10.82 mmol, 86% yield) as yellow oil.

LCMS (System A, UV, ESI): Rₜ=0.69 min, [M+H]⁺ 395, 397

Similarly prepared was:

### Intermediate 94 (S)-2-(2-((6-chloro-4-((methylsulfonyl)methyl)pyridin-2-yl)amino)butoxy)acetic acid

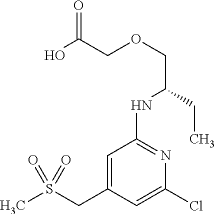 [SMILES: CC[C@@H](COCC(=O)O)Nc1cc(CS(C)(=O)=O)cc(Cl)n1]

From Intermediate 93

LCMS (SystemB, UV, ESI): Rₜ=0.80 min, [M+H]⁺ 351.1

### Intermediate 47 (S)-4-(6-bromo-4-((methylsulfonyl)methyl)pyridin-2-yl)-5-ethylmorpholin-3-one

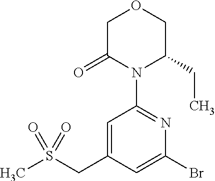 [SMILES: CC[C@H]1COCC(=O)N1c1cc(CS(C)(=O)=O)cc(Br)n1]

(S)-2-(2-((6-Bromo-4-((methylsulfonyl)methyl)pyridin-2-yl)amino)butoxy)acetic acid (3.6 g, 9.11 mmol) and HATU (4.50 g, 11.84 mmol) in N,N-Dimethylformamide (DMF) (20 mL) was added TEA (2.67 mL, 19.13 mmol) stirred at room temperature for 3 hours.

The reaction mixture was combined with a previous batch of 1 g (2.53 mmol) and the combined reaction mixture partitioned between EtOAc 300 mL and water 400 mL, and extracted with EtOAc (150 mL×4). The organic phase was dried over sodium sulphate and evaporated in vacuo to give the crude product as a brown solid.

The sample was preabsorbed on silica and purified on a silica (Si) 330 g using a 0%-50% ethyl acetate-petroleum solvent gradient over 120 mins, Flow rate: 70 mL/min. The appropriate fractions were combined and evaporated in vacuo to give crude product. The crude product was further purified by prep-HPLC [conditions: C18 column (660 g), Mobile Phase A:Water(10 MMOL/L NH4HCO3), Mobile Phase B: ACN; Flow rate: 100 mL/min; Gradient: 40% B to 45% B in 20 min;

254 nm; Rₜ: 21 min]. The appropriate fractions were combined to give about 800 mL solvent. The ACN was removed under vacuum, aqueous phase was extracted with DCM (150 mL×4). The organic phase was dried over sodium sulphate and evaporated in vacuo to give the desired product (S)-4-(6-bromo-4-((methylsulfonyl)methyl)pyridin-2-yl)-5-ethylmorpholin-3-one (2.4798 g, 6.40 mmol, 55% yield) as a off-white solid.

1H-NMR (HNMR-N67412-16-A1) (400 MHz, DMSO-d6): δ [ppm] 8.05 (s, 1H), 7.54 (s, 1H), 4.65 (s, 2H), 4.43-4.40 (m, 1H), 4.37-4.32 (s, 1H), 4.24-4.20 (d, J=17.2 Hz, 1H),

4.05-4.02 (m, 1H), 3.93-3.89 (m, 1H), 3.00 (s, 3H), 1.74-1.64 (m, 1H), 1.60-1.51 (m, 1H), 0.87-0.84 (m, 3H).

ANAL_SFC: RT=1.40 min, 99% ee.

LCMS (System A, UV, ESI): Rₜ=1.29 min, [M+H]⁺ 377, 379

Similarly prepared was:

### Intermediate 95 (S)-4-(6-chloro-4-((methylsulfonyl)methyl)pyridin-2-yl)-5-ethylmorpholin-3-one

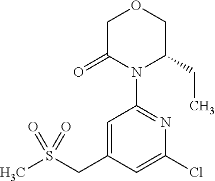 [SMILES: CC[C@H]1COCC(=O)N1c1cc(CS(C)(=O)=O)cc(Cl)n1]

From Intermediate 94

LCMS (System A, UV, ESI): Rₜ=1.29 min, [M+H]⁺ 333.1

### Intermediate 48 (S)-4-(6-bromo-4-(4-(methylsulfonyl)tetrahydro-2H-pyran-4-yl)pyridin-2-yl)-5-ethylmorpholin-3-one

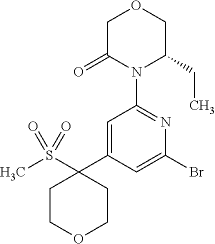 [SMILES: CC[C@H]1COCC(=O)N1c1cc(C2(S(C)(=O)=O)CCOCC2)cc(Br)n1]

(S)-4-(6-bromo-4-((methylsulfonyl)methyl)pyridin-2-yl)-5-ethylmorpholin-3-one (200 mg, 0.530 mmol), potassium carbonate (440 mg, 3.18 mmol) and 18-crown-6 (84 mg, 0.318 mmol) were sealed in a microwave vial. The vial was then vacuumed and purged with nitrogen. 1-bromo-2-(2-bromoethoxy)ethane (200 μl, 1.590 mmol) in dry N,Ndimethylformamide (DMF) (2500 μl) was then added to the vial. The reaction mixture was then stirred at 80° C. for 4 hours, cooled to room temperature and stood for 24 h.

The reaction mixture was partitioned between EtOAc (20 mL) and water (20 mL) (pH=11). The aqueous layer was back extracted with EtOAc (20 mL). The organic layers were combined, washed with brine (40 mL) then passed through a hydrophobic frit. The solvent was removed under vacuum.

The residue was then purified by column chromatography (24 g silica, EtOAc:Cy 40%-80%, 26 column volumes. The product containing fractions were combined and the

solvent removed under vacuum to yield (S)-4-(6-bromo-4-(4-methylsulfonyl) tetrahydro-2H-pyran-4-yl)pyridin-2-yl)-5-ethylmorpholin-3-one (175 mg, 0.352 mmol, 66.4%

yield) as a colourless solid.

LCMS (System A, UV, ESI): Rₜ=0.90 min, [M+H]⁺ 447

Alternatively

1-bromo-2-(2-bromoethoxy)ethane (27 μl, 0.215 mmol) was added to a solution of (S)-4-(6-bromo-4-((methylsulfonyl)methyl)pyridin-2-yl)-5-ethylmorpholin-3-one (50 mg, 0.133 mmol) and NaH (60% in mineral oil) (17.0 mg, 0.425 mmol) in N,N-Dimethylformamide (DMF) (1000 μl) under an atmosphere of nitrogen at 0° C. The reaction was stirred in a melting ice bath for 43 h.

The mixture was quenched by careful addition of sat. ammonium chloride solution (5 mL). The mixture was left to stir for 5 min. The mixture was then diluted with EtOAc (10 mL) and water (10 mL). The pH of the aqueous layer was checked and was found to be pH 7, ensuring carboxylic acid by product remained in the aqueous layer. The organic layer was washed with brine (10 mL). The organic was separated and passed through a hydrophobic frit and the solvents removed under vacuum.

The crude product was then purified by MDAP (HPH, Method B). The product containing fractions were combined and the solvent removed under vacuum to yield (S)-4-(6-bromo-4-(4-(methylsulfonyl) tetrahydro-2H-pyran-4-yl)pyridin-2-yl)-5-ethylmorpholin-3-one (17.4 mg, 0.039 mmol, 29.3% yield) as a colourless gum.

LCMS (System A, UV, ESI): Rₜ=0.92 min, [M+H]⁺ 447

### Intermediate 49 1-(2,6-dibromopyrimidin-4-yl)-N,N-dimethylmethanesulfonamide

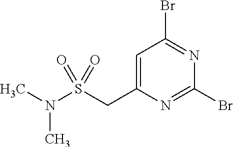

To a solution of N,N-dimethylmethanesulfonamide (99 mg, 0.805 mmol) in Tetrahydrofuran (THF) (1.5 mL) under nitrogen at 0° C. was added n-butyllithium (0.4 mL, 0.840 mmol). This solution was then added dropwise to a solution of 2,4,6-tribromopyrimidine (102 mg, 0.322 mmol) in 1.5 mL THF. The reaction mixture was allowed to warm to rt and stirred for 10 min.

The reaction mixture was quenched with 2 mL sat. NH₄Cl solution, then partitioned between 4 more mL sat. NH₄Cl solution and 6 mL EtOAc. The organic layer was separated, dried over a hydrophobic frit, and concentrated under a stream of nitrogen. The residue was dissolved in 1 mL DCM and eluted on a 12 g silica gel column in cyclohexane with a gradient of 0-50% EtOAc over 20 cv. The desired fractions were concentrated under a stream of nitrogen to give 1-(2,6-dibromopyrimidin-4-yl)-N,N dimethylmethanesulfonamide (30 mg, 0.084 mmol, 26.0% yield) as a white solid.

LCMS (System A, UV, ESI): Rₜ=0.92 min, [M+H]⁺ 356.1, 358.1, 360.1

### Intermediate 50 1-{2-bromo-6-[(3S)-3-ethylmorpholin-4-yl]pyrimidin-4-yl}-N,N-dimethylmethanesulfonamide

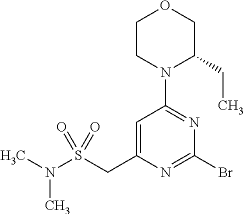 [SMILES: CC[C@H]1COCCN1c1cc(CS(=O)(=O)N(C)C)nc(Br)n1]

A solution of (S)-3-ethylmorpholine hydrochloride (75 mg, 0.495 mmol) and DIPEA (200 μl, 1.145 mmol) in 1 mL DMSO was added to a solution of 1-(2,6-dibromopyrimidin-4-yl)-N,Ndimethylmethanesulfonamide (160 mg, 0.446 mmol) in 1 mL DMSO. The reaction mixture was stirred at rt for 2 h. (S)-3-ethylmorpholine hydrochloride (9 mg, 0.059 mmol) was added and the reaction mixture was heated to 50° C. and stirred for 30 min.

The reaction mixture was diluted with 2 mL water and the resultant mixture was partitioned between 8 mL DCM and 6 more mL water. The organic layer was taken, dried over a hydrophobic frit, and concentrated under a stream of nitrogen. The residue was redissolved in 8 mL DCM and washed with 8 mL brine. The organic layer was taken, dried over a hydrophobic frit, and concentrated under a stream of nitrogen and reduced pressure to give (S)-1-(2-bromo-6-(3-ethylmorpholino)pyrimidin-4-yl)-N,N-dimethylmethanesulfonamide (188 mg, 0.478 mmol, 107% yield) as a brown gummy oil.

LCMS (System A, UV, ESI): Rₜ=1.01 min, [M+H]⁺ 393.0, 394.9

### Intermediate 51 2,6-dibromo-4-(((tert-butyldiphenylsilyl)oxy)methyl)pyridine

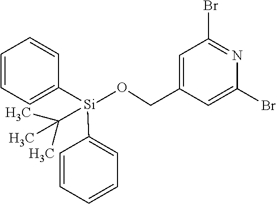 [SMILES: CC(C)(C)[Si](OCc1cc(Br)nc(Br)c1)(c1ccccc1)c1ccccc1]

Tert-butylchlorodiphenylsilane (1.508 mL, 5.80 mmol) and (2,6-dibromopyridin-4-yl)methanol (909 mg, 3.41 mmol) were dissolved in Dichloromethane (DCM) (30 mL) and imidazole (0.515 g, 7.56 mmol) was added slowly. The reaction mixture was stirred under Nitrogen flow for 1 h 30 mins.

The mixture was then partitioned with 40 mL brine. The organic layer was dried over an hydrophobic frit and concentrated. The residue was dissolved in 3 mL DCM and eluted on 120 g silica column with cyclohexane 0-20% ethyl acetate over 10CV. The desired fractions were concentrated to give 2,6-dibromo-4-(((tert-butyldiphenylsilyl)oxy)methyl) pyridine (1.4149 g, 2.66 mmol, 78% yield) as a colourless oil.

LCMS (System A, UV, ESI): Rₜ=1.77 min, [M+H]⁺ 504.0, 506.1, 507.9.

### Intermediate 52 (S)-2-((6-bromo-4-(((tert-butyldiphenylsilyloxy)methyl)pyridin-2-yl)amino)butan-1-ol

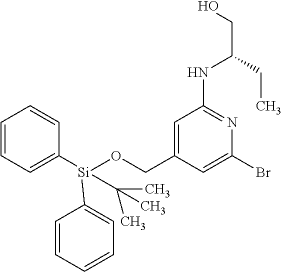 [SMILES: CC[C@@H](CO)Nc1cc(CO[Si](c2ccccc2)(c2ccccc2)C(C)(C)C)cc(Br)n1]

To a solution of 2,6-dibromo-4-(((tert-butyldiphenylsilyl)oxy)methyl)pyridine (1.3277 g, 2.63 mmol) in 2,2,6,6-tetramethylpiperidine (3.6 ml, 21.33 mmol) was added (S)-2-aminobutan-1-ol (0.300 ml, 3.18 mmol). Vial was sealed and stirred at 120° C. for 2 hr. (S)-2-aminobutan-1-ol (0.250 ml, 2.65 mmol) was added after cooling down of the vial. Microwave vial was sealed and the mixture was stirred at 150 degrees for 19 h.

(S)-2-aminobutan-1-ol (0.300 ml, 3.18 mmol) was added and mixture stirred at 150 degrees for 70 h.

The reaction mixture was partitioned between 10 mL ethyl acetate, 10 mL of water. The organic layer was dried on a hydrophobic frit and concentrated under reduced pressure. The residue was eluted on 80 g normal phase silica with cyclohexane and 0-40% Ethyl acetate over 12CV. The desired fractions were concentrated to give (S)-2-((6-bromo-4-(((tert-butyldiphenylsilyl)oxy)methyl)pyridin-2-yl)amino)butan-1-ol (1.0949 g, 2.132 mmol, 81% yield) as a colourless oil.

LCMS (System A, UV, ESI): Rₜ=1.64 min, [M+H]⁺ 513.2, 515.2

### Intermediate 53 ethyl (S)-2-(2-((6-bromo-4-(((tert-butyldiphenylsilyl)oxy)methyl)pyridin-2-yl)amino)butoxy)acetate

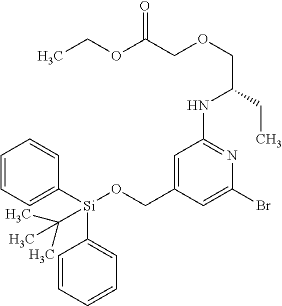 [SMILES: CCOC(=O)COC[C@H](CC)Nc1cc(CO[Si](c2ccccc2)(c2ccccc2)C(C)(C)C)cc(Br)n1]

Ethyl 2-diazoacetate (0.250 mL, 2.068 mmol) in solution in Dichloromethane (DCM) (10 mL) was added over 2 h using an addition funnel to a solution of (S)-2-((6-bromo-4-(((tertbutyldiphenylsilyl)oxy)methyl)pyridin-2-yl)amino)butan-1-ol (662.5 mg, 1.290 mmol) and diacetoxyrhodium (45.7 mg, 0.103 mmol) in Dichloromethane (DCM) (1.2 mL) at reflux under an atmosphere of nitrogen. The resulting mixture was stirred at reflux for 30 mins.

The mixture was concentrated under reduced pressure and partitioned between 15 mL DCM and 2×15 mL saturated ammonium chloride aqueous solution. Organic layer was dried over an hydrophobic frit and concentrated under reduce pressure. The residue was dissolved in 4 mL DCM and eluted on 60 g silica gel NP with gradient cyclohexane 0-70% Ethyl acetate over 12CV. The desired fraction were concentrated under reduced pressure to give ethyl (S)-2-(2-((6-bromo-4-(((tert-butyldiphenylsilyl)oxy)methyl)pyridin-2-yl)amino)butoxy)acetate (833 mg, 1.389 mmol, 108% yield).

LCMS (System A, UV, ESI): Rₜ=1.76 min, [M+H]⁺ 475.3, 476.3

### Intermediate 54 (S)-2-(2-((6-bromo-4-(((tert-butyldiphenylsilyl)oxy)methyl)pyridin-2-yl)amino)butoxy)acetic acid

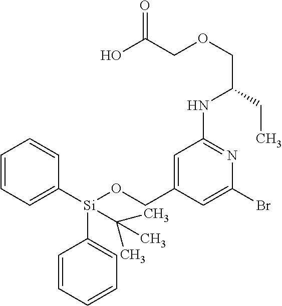 [SMILES: CC[C@@H](COCC(=O)O)Nc1cc(CO[Si](c2ccccc2)(c2ccccc2)C(C)(C)C)cc(Br)n1]

To a solution of ethyl (S)-2-(2-((6-bromo-4-(((tert-butyldiphenylsilyl)oxy) methyl)pyridin-2-yl)amino)butoxy)acetate (830 mg, 1.384 mmol) in Tetrahydrofuran (THF) (10 mL) was added aqueous sodium hydroxide solution 2M (3 mL, 3.00 mmol), the reaction mixture was stirred for 2.5 hr at RT.

55 Drops of HCl 1M were added to the reaction mixture (until pH 1-2) that was then partitioned with 25 mL saturated aqueous ammonium chloride solution. The organic layer was dried on an hydrophobic frit and concentrated under nitrogen flow to give (S)-2-(2-((6-bromo-4-(((tert-butyldiphenylsilyl) oxy)methyl)pyridin-2-yl)amino)butoxy)acetic acid (523 mg, 0.915 mmol, 66.1% yield) as a yellow gum.

LCMS (System A, UV, ESI): Rₜ=1.22 min, [M+H]⁺ 571.2, 573.2

### Intermediate 55 (S)-4-(6-bromo-4-(((tert-butyldiphenylsilyloxy)methylpyridin-2-yl)-5-ethylmorpholin-3-one

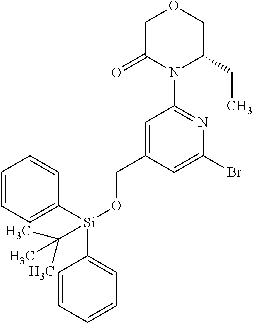 [SMILES: CC[C@H]1COCC(=O)N1c1cc(CO[Si](c2ccccc2)(c2ccccc2)C(C)(C)C)cc(Br)n1]

Triethylamine (0.254 mL, 1.819 mmol) was added to a mixture of (S)-2-(2-((6-bromo-4-(((tert-butyldiphenylsilyl)oxy)methyl)pyridin-2-yl)amino)butoxy)acetic acid (520 mg, 0.910 mmol) and HATU (713 mg, 1.875 mmol) in dry N,N-Dimethylformamide (DMF) (9 mL) under a nitrogen atmosphere at RT in a sealed microwave vial. This mixture was stirred at RT for 1.5 hr.

DMF was azeotroped with 2×10 mL of toluene and concentrated under reduced pressure.

The product was dry loaded on 40 g silica column with cyclohexane 0-50% Ethyl Acetate over 12CV. The desired fractions were concentrated to give (S)-4-(6-bromo-4-(((tertbutyldiphenylsilyl)oxy)methyl)pyridin-2-yl)-5-ethylmorpholin-3-one (347 mg, 0.627 mmol, 68.9% yield) as a transparent oil.

LCMS (System A, UV, ESI): Rₜ=1.73 min, [M+H]⁺ 553.2, 555.2

### Intermediate 56 (S)-4-(6-bromo-4-(hydroxymethyl)pyridin-2-yl)-5-ethylmorpholin-3-one

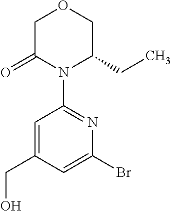 [SMILES: CC[C@H]1COCC(=O)N1c1cc(CO)cc(Br)n1]

TBAF 1.0M in THF (0.752 mL, 0.752 mmol) was added to a solution of (S)-4-(6-bromo-4-(((tert-butyldiphenylsilyl)oxy)methyl)pyridin-2-yl)-5-ethylmorpholin-3-one (347 mg, 0.627 mmol) in dry Tetrahydrofuran (THF) (3 mL) and stirred 30 min in a sealed microwave vial at RT.

Reaction mixture was partitioned with 10 mL saturated aqueous sodium bicarbonate solution and aqueous layer was back extracted with 2×10 mL Ethyl acetate. Organic layer was dried over an hydrophobic frit and concentrated under reduce pressure. The residue was eluted on 40 g silica gel gradient cyclohexane 0-100% Ethyl acetate over 12 CV. The desired fractions were concentrated under reduce pressure to give (S)-4-(6-bromo-4-(hydroxymethyl)pyridin-2-yl)-5-ethylmorpholin-3-one (191 mg, 0.606 mmol, 97% yield) as a colourless oil.

LCMS (System A, UV, ESI): Rₜ=0.82 min, [M+H]⁺ 315.0, 317.0

### Intermediate 57 (S)-4-(6-bromo-4-(bromomethyl)pyridin-2-yl)-5-ethylmorpholin-3-one

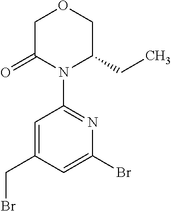 [SMILES: CC[C@H]1COCC(=O)N1c1cc(CBr)cc(Br)n1]

To a solution of (S)-4-(6-bromo-4-(hydroxymethyl)pyridin-2-yl)-5-ethylmorpholin-3-one (230 mg, 0.730 mmol) in Tetrahydrofuran (THF) (5 mL), triphenylphosphine (232 mg, 0.885 mmol)

and then 1-bromopyrrolidine-2,5-dione (154 mg, 0.865 mmol) were added at room temperature, and the mixture was stirred at RT for 1 hr.

The reaction mixture was then partitioned between 15 mL saturated aqueous sodium bicarbonate solution and 15 mL ethyl acetate. The organic layer was taken, dried over a hydrophobic frit, and concentrated under reduced pressure. The residue was dissolved in 1.5 mL DCM, and eluted on a 24 g silica gel column in cyclohexane with a gradient of 0-50% EtOAc over 12CV. The desired fractions were concentrated under reduced pressure to give (S)-4-(6-bromo-4-(bromomethyl)pyridin-2-yl)-5-ethylmorpholin-3-one (147 mg, 0.389 mmol, 53.3% yield) as a colourless oil.

LCMS (System A, UV, ESI): Rₜ=1.17 min, [M+H]⁺ 377.0, 379.0, 381.0

### Intermediate 58 methyl 3-({2-chloro-6-[(3S)-3-ethylmorpholin-4-yl]pyrimidin-4-yl}methanesulfonyl)propanoate

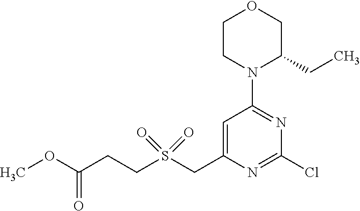 [SMILES: CC[C@H]1COCCN1c1cc(CS(=O)(=O)CCC(=O)OC)nc(Cl)n1]

(S)-4-(6-(bromomethyl)-2-chloropyrimidin-4-yl)-3-ethylmorpholine (2447 mg, 7.63 mmol), sodium 3-methoxy-3-oxopropane-1-sulfinate (1500 mg, 8.61 mmol), and 30 mL MeCN were split equally across 2 microwave vials which were sealed and heated at 90° C. for 2 h.

The reaction mixture was then partitioned between 70 mL water and 70 mL EtOAc. The organic layer was removed, and the aqueous layer was back-extracted with 70 mL EtOAc. The organic layers were combined, dried over a hydrophobic frit, and concentrated under reduced pressure to give methyl (S)-3-(((2-chloro-6-(3-ethylmorpholino)pyrimidin-4-yl)methyl)sulfonyl)propanoate (2087 mg, 5.33 mmol, 69.8% yield) as a yellow gum.

LCMS (System A, UV, ESI): Rₜ=0.95 min, [M+H]⁺ 392, 394

Similarly prepared was:

### Intermediate 59 methyl (S)-3-(((2-bromo-6-(3-ethylmorpholino)pyridin-4-yl)methyl)sulfonyl)propanoate

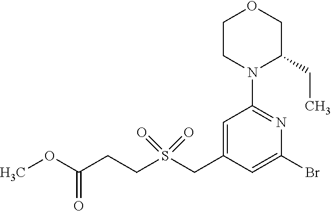 [SMILES: CC[C@H]1COCCN1c1cc(CS(=O)(=O)CCC(=O)OC)cc(Br)n1]

From Intermediate 35

LCMS (System A, UV, ESI): Rₜ=1.10 min, [M+H]⁺ 435.2, 437.2

Similarly prepared was:

### Intermediate 60 methyl (S)-3-(((2-bromo-6-(3-ethyl-5-oxomorpholino)pyridin-4-yl)methyl)sulfonyl)propanoate

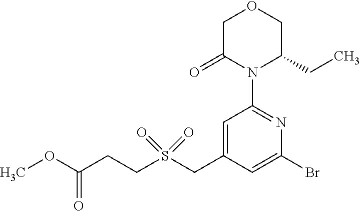 [SMILES: CC[C@H]1COCC(=O)N1c1cc(CS(=O)(=O)CCC(=O)OC)cc(Br)n1]

From Intermediate 57

LCMS (System A, UV, ESI): Rₜ=0.96 min, [M+H]⁺ 449.1, 451.1

### Intermediate 61 sodium {2-chloro-6-[(3S)-3-ethylmorpholin-4-yl]pyrimidin-4-yl}methanesulfinate

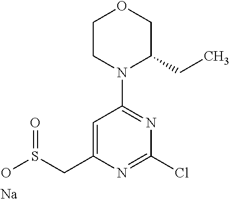

Sodium methoxide (3.01 ml, 1.503 mmol) was added dropwise to methyl (S)-3-(((2-chloro-6-(3-ethylmorpholino)pyrimidin-4-yl)methyl)sulfonyl)propanoate (620 mg, 1.582 mmol) in Tetrahydrofuran (THF) (10 ml). The reaction mixture was stood at RT for 5 min.

The reaction mixture was concentrated under reduced pressure to give (S)-(2-chloro-6-(3-ethylmorpholino) pyrimidin-4-yl)methanesulfinate, Sodium salt (161 mg, 0.442 mmol, 98% yield) as a light brown solid.

LCMS (System A, UV, ESI): Rₜ=0.57 min, [M+H]⁺ 304, 306.

Similarly prepared was:

### Intermediate 62 (S)-(2-bromo-6-(3-ethylmorpholino)pyridin-4-yl)methanesulfinate, Sodium salt

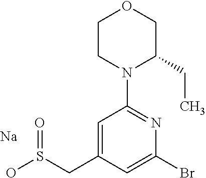

From Intermediate 59

LCMS (System A, UV, ESI): Rₜ=0.68 min, [M+H]⁺ 349.0, 351.0

### Intermediate 63 (S)-(2-bromo-6-(3-ethyl-5-oxomorpholino)pyridin-4-yl)methanesulfinate, Sodium salt

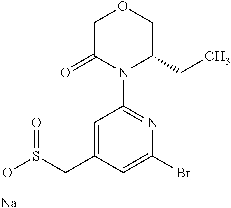

From Intermediate 60

LCMS (System A, UV, ESI): Rₜ=0.56 min, [M+H]⁺ 299.1, 301.0

### Intermediate 64 1-{2-chloro-6-[(3S)-3-ethylmorpholin-4-yl]pyrimidin-4-yl}-N,N-dimethylmethanesulfonamide

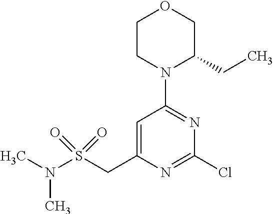 [SMILES: CC[C@H]1COCCN1c1cc(CS(=O)(=O)N(C)C)nc(Cl)n1]

A solution of sodium (S)-(2-chloro-6-(3-ethylmorpholino)pyrimidin-4-yl)methanesulfinate (510 mg, 1.556 mmol) and dimethylamine 2M in THF (1.167 mL, 2.334 mmol) in dry Tetrahydrofuran (THF) (2.000 mL) and Dimethyl Sulfoxide (DMSO) (0.3 mL) was added dropwise to a solution of iodine (434 mg, 1.712 mmol) in dry Tetrahydrofuran (THF) (2 mL). The reaction mixture was stirred at 21° C. for 45 min. Further dimethylamine 2M in THF (0.195 mL, 0.389 mmol) was added and the reaction mixture stirred for a further 30 minutes.

The reaction mixture was quenched with 0.5 mL 28% sodium thiosulfate solution then partitioned between 10 mL water and 10 mL EtOAc. The organic phase was filtered through a hydrophobic frit, and concentrated in vacuo. The sample was loaded in dichloromethane and purified on silica (Si) 50 g and eluted using a 0-100% ethyl acetate-cyclohexane gradient over 30 mins. The appropriate fractions were combined and evaporated in vacuo to give the required product 1-{2-chloro-6-[(3S)-3-ethylmorpholin-4-yl]pyrimidin-4-yl}-N,N-dimethylmethanesulfonamide, 273 mg as a yellow gum.

LCMS (System A, UV, ESI): Rₜ=0.96 min, [M+H]⁺ 349.

Similarly prepared was:

### Intermediate 96 (S)-1-(2-bromo-6-(3-ethyl-5-oxomorpholino)pyridin-4-yl)-N,Ndimethyl-methanesulfonamide

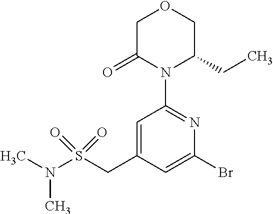 [SMILES: CC[C@H]1COCC(=O)N1c1cc(CS(=O)(=O)N(C)C)cc(Br)n1]

From Intermediate 63

LCMS (System A, UV, ESI): Rₜ=1.00 min, [M+H]⁺ 406., 408.1

### Intermediate 65 1-{2-chloro-6-[(3S)-3-ethylmorpholin-4-yl]pyrimidin-4-yl}-N-[(4-methoxyphenyl)methyl]methanesulfonamide

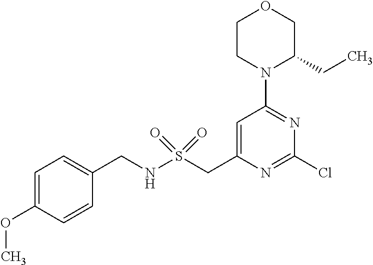 [SMILES: CC[C@H]1COCCN1c1cc(CS(=O)(=O)NCc2ccc(OC)cc2)nc(Cl)n1]

A solution of iodine (413 mg, 1.627 mmol) in 1 mL THF was added to a solution of (4-methoxyphenyl)methanamine (950 mg, 6.93 mmol) and sodium (S)-(2-chloro-6-(3-ethylmorpholino)pyrimidin-4-yl)methanesulfinate (447 mg, 1.364 mmol) in 7 mL THF. The reaction mixture was stood at rt for 5 min.

The reaction mixture was quenched with 3 mL 5% sodium metabisulfite solution then partitioned between 20 mL brine and 20 mL EtOAc. The organic layer was taken, dried over a hydrophobic frit, and concentrated under reduced pressure. The residue was dissolved in 2 mL DCM and eluted on a 40 g silica gel column in cyclohexane with a gradient of 0-60% EtOAc over 16 cv. The desired fractions were concentrated under reduced pressure to give (S)-1-(2-chloro-6-(3-ethylmorpholino)pyrimidin-4-yl)-N-(4-methoxybenzyl)methanesulfonamide (418 mg, 0.948 mmol, 69.5% yield) as a light pink foam.

LCMS (System A, UV, ESI): Rₜ=1.13 min, [M+H]⁺ 441.2, 443.2.

Similarly prepared was:

### Intermediate 66 (S)-1-(2-bromo-6-(3-ethylmorpholino)pyridin-4-yl)-N-(4-methoxybenzyl) methanesulfonamide

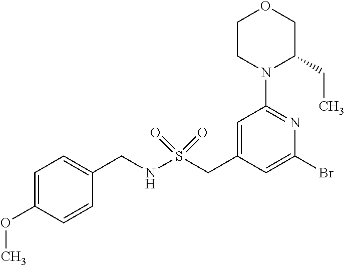 [SMILES: CC[C@H]1COCCN1c1cc(CS(=O)(=O)NCc2ccc(OC)cc2)cc(Br)n1]

From (4-methoxyphenyl) methanamine (656 mg, 4.78 mmol) and Intermediate 62

LCMS (System A, UV, ESI): Rₜ=1.24 min, [M+H]⁺ 484.1, 486.0

### Intermediate 67 N-[(4-methoxyphenyl)methyl]-N-methylmethanesulfonamide

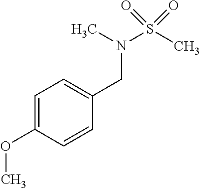

To a solution of 1-(4-methoxyphenyl)-N-methylmethanamine (1008 mg, 6.67 mmol) and pyridine (0.6 mL, 7.42 mmol) in Tetrahydrofuran (THF) (30 mL) at 0° C. was added methanesulfonyl chloride (0.460 mL, 5.94 mmol) dropwise. The reaction mixture was stirred at 0° C. for 1.5 h.

The reaction mixture was partitioned between 70 mL 5% citric acid solution and 100 mL EtOAc. The organic layer was taken, dried over a hydrophobic frit, and concentrated under reduced pressure. The residue was dissolved in methanol, ˜5 g florisil was added, and the reaction mixture was concentrated under reduced pressure. The residue was then dry loaded onto a 80 g silica gel column and eluted with cyclohexane with a gradient of 0-100% EtOAc over 12 cv. The desired fractions were concentrated under reduced pressure to give N-(4-methoxybenzyl)-N-methylmethanesulfonamide (695 mg, 3.03 mmol, 45.5% yield) as a white solid.

¹H NMR (400 MHz, CHLOROFORM-d) δ 7.29-7.32 (m, 2H), 6.88-6.95 (m, 2H), 4.27 (s, 2H), 3.84 (s, 3H), 2.83 (s, 3H), 2.77 (s, 3H), 1.28 (t, J=7.21 Hz, 3H)

### Intermediate 68 1-(2,6-dichloropyrimidin-4-yl)-N-[(4-methoxyphenyl)methyl]-N-methylmethanesulfonamide

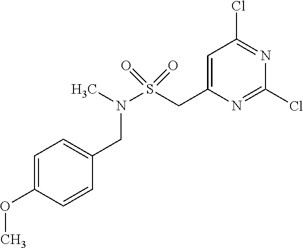 [SMILES: COc1ccc(CN(C)S(=O)(=O)Cc2cc(Cl)nc(Cl)n2)cc1]

To a solution of N-(4-methoxybenzyl)-N-methylmethanesulfonamide (695 mg, 3.03 mmol) in Tetrahydrofuran (THF) (6 mL) in a round bottomed flask under nitrogen at 0° C. was added n-butyllithium (1.5 mL, 3.15 mmol). This solution was then added dropwise to a solution of 2,4,6-trichloropyrimidine (220 mg, 1.199 mmol) in 6 mL THF. The reaction mixture was allowed to warm to rt and stirred at 0° C. for 5 min, then quenched with 6 mL sat. NH₄Cl solution and stirred vigorously for 5 min.

The reaction mixture was partitioned between 50 more mL sat. NH₄Cl solution and 50 mL EtOAc. The organic layer was separated, dried over a hydrophobic frit, and concentrated under reduced pressure. The residue was dissolved in 3 mL DCM and eluted on a 40 g silica gel column in cyclohexane with a gradient of 0-50% EtOAc over 20 cv. Collected fractions were concentrated under reduced pressure to give 1-(2,6-dichloropyrimidin-4-yl)-N-(4-methoxybenzyl)-N-methylmethanesulfonamide (283 mg, 0.752 mmol, 62.7% yield) as an off-white gum.

LCMS (System A, UV, ESI): Rₜ=1.17 min, [M+H]⁺ 374.1

### Intermediate 69 1-{2-chloro-6-[(3S)-3-ethylmorpholin-4-yl]pyrimidin-4-yl}-N-[(4-methoxyphenyl)methyl]-N-methylmethanesulfonamide

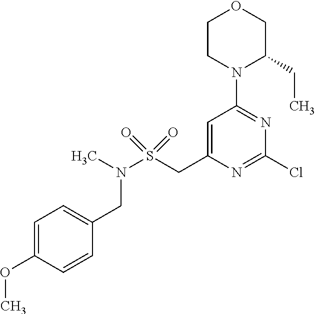 [SMILES: CC[C@H]1COCCN1c1cc(CS(=O)(=O)N(C)Cc2ccc(OC)cc2)nc(Cl)n1]

DIPEA (175 μl, 1.002 mmol) was added to a solution of 1-(2,6-dichloropyrimidin-4-yl)-N-(4-methoxybenzyl)-N-methylmethanesulfonamide (283 mg, 0.752 mmol) and (S)-3-ethylmorpholine hydrochloride (135 mg, 0.890 mmol) in 3 mL DMSO. The reaction mixture was stirred at rt for 2 h, then (S)-3-ethylmorpholine hydrochloride (45 mg, 0.297 mmol) and DIPEA (70 μl, 0.401 mmol) were added, the reaction mixture was heated to 50° C., and stirred for 2.5 h, then cooled to rt and stood for 16 h.

The reaction mixture was partitioned between 20 mL EtOAc and 20 mL brine. The organic layer was taken, dried over a hydrophobic frit, and concentrated under reduced pressure. The residue was dissolved in 2 mL DCM and eluted on a 40 g silica gel column in cyclohexane with a gradient of 0-50% EtOAc over 30 cv. This failed to separate the two regioisomers so the desired fractions were combined, diluted with 1 mL DMSO, and concentrated under reduced pressure. The residue was taken, diluted with another 1 mL DMSO, and eluted on an Xselect column in 10 mM ammonium bicarbonate with a gradient of 50-99% MeCN over 50 minutes. This also failed to separate the regioisomers. Collected fractions were submitted to LCMS and the desired fractions were concentrated under reduced pressure to give 1-{2-chloro-6-[(3S)-3-ethylmorpholin-4-yl]pyrimidin-4-yl}-N-[(4-methoxyphenyl)methyl]-N-methylmethanesulfonamide as a yellow gum (mixture of regioisomers)

LCMS (System A, UV, ESI): Rₜ=1.22 min, [M+H]⁺ 455.1

### Intermediate 70 (S)-4-(6-bromo-4-(4-(methylsulfonylpiperidin-4-yl)pyridin-2-yl)-3-ethylmorpholine

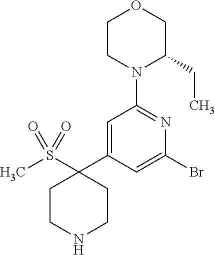 [SMILES: CC[C@H]1COCCN1c1cc(C2(S(C)(=O)=O)CCNCC2)cc(Br)n1]

Tert-butyl (S)-4-(2-bromo-6-(3-ethylmorpholino)pyridin-4-yl)-4-(methylsulfonyl)piperidine-1-carboxylate (741 mg, 1.392 mmol) was suspended in HCl 4M in Dioxane (3479 μl, 13.92 mmol) and sonicated for 2 mins to aid dissolution. The reaction mixture was stirred for 30 mins at 21° C. and concentrated by blowdown. The residue was suspended in MeOH (500 uL) and applied to a preconditioned 5 g NH₂ column. The product was eluted with MeOH (2 column volumes) and concentrated by blowdown to afford (S)-4-(6-bromo-4-(4-(methylsulfonyl)piperidin-4-yl)pyridin-2-yl)-3-ethylmorpholine.

LCMS (SystemB, UV, ESI): Rₜ=0.61 min, [M+H]⁺ 432

### Intermediate 71 (S)-4-(6-bromo-4-(1-methyl-4-(methylsulfonyl)piperidin-4-yl)pyridin-2-yl)-3-ethylmorpholine

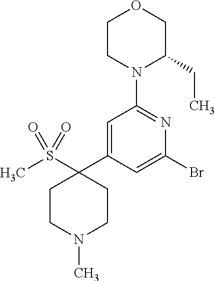 [SMILES: CC[C@H]1COCCN1c1cc(C2(S(C)(=O)=O)CCN(C)CC2)cc(Br)n1]

Formaldehyde (393 μl, 5.27 mmol) was added to dropwise to a solution of (S)-4-(6-bromo-4-(4-(methylsulfonyl)piperidin-4-yl)pyridin-2-yl)-3-ethylmorpholine (570 mg, 1.318 mmol) in formic acid (1517 μl, 39.5 mmol). The reaction mixture was heated under nitrogen at 90° C. for 1 h. The reaction mixture was partitioned between EtOAc (10 mL) and saturated Sodium bicarbonate solution (10 mL). The organic phase was washed with Brine (10 mL), passed through a Hydrophobic frit and concentrated in vacuo. The residue was purified on silica (Si) 50 g and eluted using a 0-100% ethyl acetate-cyclohexane and Ethyl acetate to 50% EtOH in EtOAc+1% Et3N gradient over 60 mins. The appropriate fractions were combined and evaporated in vacuo to give (S)-4-(6-bromo-4-(1-methyl-4-(methylsulfonyl) piperidin-4-yl)pyridin-2-yl)-3-ethylmorpholine (233 mg) as a yellow gum.

LCMS (System B, UV, ESI): Rₜ=0.60 min, [M+H]⁺ 446

### Intermediate 72 methyl (S)-3-((6-(3-ethylmorpholino)-4-((methylsulfonyl)methyl) pyridin-2-yl)sulfonyl)propanoate

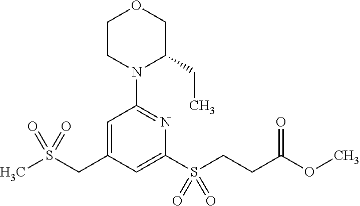

(S)-4-(6-Bromo-4-((methylsulfonyl)methyl)pyridin-2-yl)-3-ethylmorpholine (3.56 g, 9.80 mmol), copper(I) iodide (5.60 g, 29.4 mmol) and 3-methoxy-3-oxopropane-1-sulfinate, Sodium salt (3.5 g, 20.10 mmol) were placed in Dimethyl Sulfoxide (DMSO) (100 mL) and degassed under N2 gas for 20 minutes. The resulting reaction mixture was heated to 110° C. under flow of N2 gas for 1 hour. EtOAc (100 mL) was added to the cooled reaction mixture. The resulting mixture was filtered on Celite (10 g) and washed through with EtOAc (2×100 mL). The filtrate was washed with water, aqueous saturated NaHCO₃, aqueous saturated ammonium chloride (3×500 mL 2:2:1). The aqueous layers were washed separately with EtOAc (500 mL) and the organic layers were collected. The organic layers were combined, filtered through a hydrophobic frit and concentrated in vacuo to give a yellow oily residue.

The residue was purified by column chromatography on Silica (120 g column, wet load in DCM) using the elution gradient Ethyl acetate in cyclohexane 60-100% to give methyl (S)-3-((6-(3-ethylmorpholino)-4-((methylsulfonyl)methyl)pyridin-2-yl)sulfonyl)propanoate (3.69 g, 8.49 mmol, 87% yield) as a colourless oil.

LCMS (System B, UV, ESI): Rₜ=0.80 min, [M+H]⁺ 435.3

### Intermediate 73 (S)-6-(3-ethylmorpholino)-4-((methylsulfonyl)methylpyridine-2-sulfinate, Sodium salt

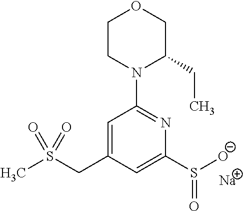 [SMILES: CC[C@H]1COCCN1c1cc(CS(C)(=O)=O)cc(S(=O)[O-])n1 ; [Na+]]

To a solution of methyl (S)-3-((6-(3-ethylmorpholino)-4-((methylsulfonyl)methyl)pyridin-2-yl)sulfonyl)propanoate (3.69 g, 8.49 mmol) in anhydrous Tetrahydrofuran (THF) (60 ml) under N2 was added sodium methoxide (0.5 M in methanol) (17.15 ml, 8.58 mmol) dropwise while stirring at room temperature. The reaction mixture was left to stir for 40 minutes.

MeOH (5 mL) was added to the reaction mixture and the volatiles were removed under reduced pressure to give (S)-6-(3-ethylmorpholino)-4-((methylsulfonyl)methyl)pyridine-2-sulfinate, Sodium salt (3.31 g, 8.94 mmol, 105% yield) as an orange solid.

LCMS (System B, UV, ESI): Rₜ=0.63 min, [M+H]⁺ 349.1.

### Intermediate 74 tert-butyl N-[(5-{4-[(3S)-3-ethylmorpholin-4-yl]-6-(methanesulfonylmethyl)pyrimidin-2-yl}-1H-pyrrolo[3,2-b]pyridin-2-yl)methyl]-N-methylcarbamate

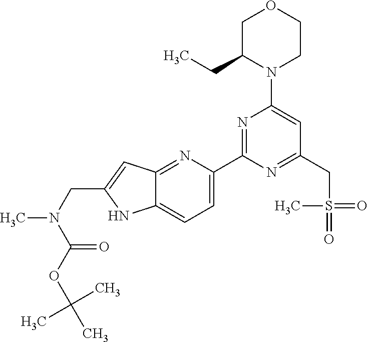 [SMILES: CC[C@H]1COCCN1c1cc(CS(C)(=O)=O)nc(-c2ccc3[nH]c(CN(C)C(=O)OC(C)(C)C)cc3n2)n1]

A solution of (S)-4-(2-chloro-6-((methylsulfonyl)methyl)pyrimidin-4-yl)-3-ethylmorpholine (50.2 mg, 0.157 mmol), sodium 2-(((tert-butoxycarbonyl)(methyl)amino)methyl)-1Hpyrrolo[3,2-b]pyridine-5-sulfinate (61.9 mg, 0.178 mmol), K2CO3 (43.4 mg, 0.314 mmol), palladium(II) acetate (8.5 mg, 0.038 mmol) and tricyclohexylphosphine (14.3 mg, 0.051 mmol) in 1,4-Dioxane (1500 μl) was degassed for 10 minutes. The reaction mixture was then sealed, heated to 150° C. and stirred at 150° C. for 2.5 h.

The reaction mixture was partitioned between DCM (30 ml) and water (30 ml). The aqueous phases were further extracted with DCM (2×30 ml) The crude product was purified by reverse phase chromatography on a XBridge Prep C18 column (80 g) using the elution gradient acetonitrile in 10 mM ammonium bicarbonate 30 to 85% to yield tert-butyl-(S)-((5-(4-(3-ethylmorpholino)-6-((methylsulfonyl)methyl)pyrimidin-2-yl)-1H-pyrrolo[3,2-b]pyridin-2-yl)methyl)(methyl)carbamate (42 mg, 0.077 mmol, 49.1% yield).

LCMS (System B, UV, ESI): Rₜ=0.75 min, [M+H]⁺ 545.3

Similarly prepared were:

### Intermediate 75 (3S)-3-ethyl-4-[6-(methanesulfonylmethyl)-2-[2-({[tris(propan-2-yl)silyl]oxy}methyl)-1H-pyrrolo[3,2-b]pyridin-5-yl]pyrimidin-4-yl]morpholine

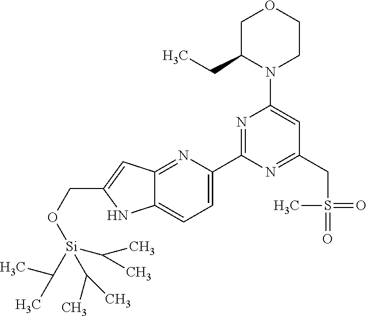 [SMILES: CC[C@H]1COCCN1c1cc(CS(C)(=O)=O)nc(-c2ccc3[nH]c(CO[Si](C(C)C)(C(C)C)C(C)C)cc3n2)n1]

From Intermediate 20 and Intermediate 36

LCMS (System A, UV, ESI): Rₜ=1.45 min, [M+H]⁺ 588.41

### Intermediate 76 tert-butyl N-[(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl]-N-methylcarbamate

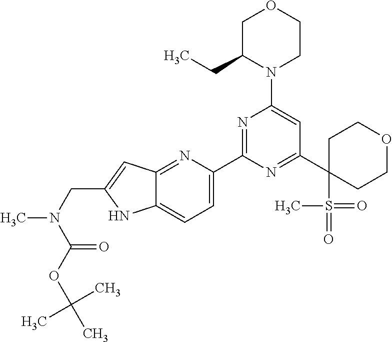 [SMILES: CC[C@H]1COCCN1c1cc(C2(S(C)(=O)=O)CCOCC2)nc(-c2ccc3[nH]c(CN(C)C(=O)OC(C)(C)C)cc3n2)n1]

From Intermediate 19 and Intermediate 39

LCMS (System B, UV, ESI): Rₜ=0.78 min, [M+H]⁺ 615.4

### Intermediate 77 (3S)-3-ethyl-4-[6-(4-methanesulfonyloxan-4-yl)-2-[2-({[tris(propan-2-yl)silyl]oxy}methyl)-1H-pyrrolo[3,2-b]pyridin-5-yl]pyrimidin-4-yl]morpholine

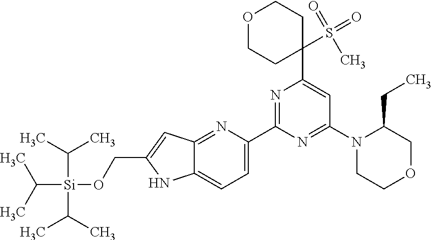 [SMILES: CC[C@H]1COCCN1c1cc(C2(S(C)(=O)=O)CCOCC2)nc(-c2ccc3[nH]c(CO[Si](C(C)C)(C(C)C)C(C)C)cc3n2)n1]

From Intermediate 20 and Intermediate 39

LCMS (System B, UV, ESI): Rₜ=0.78 min, [M+H]⁺ 615.4

### Intermediate 78 (5S)-5-ethyl-4-[6-(methanesulfonylmethyl)-2-[2-({[tris(propan-2-yl)silyl]oxy}methyl)-1H-pyrrolo[3,2-b]pyridin-5-yl]pyrimidin-4-yl]morpholin-3-one

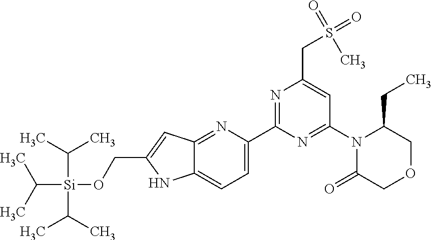 [SMILES: CC[C@H]1COCC(=O)N1c1cc(CS(C)(=O)=O)nc(-c2ccc3[nH]c(CO[Si](C(C)C)(C(C)C)C(C)C)cc3n2)n1]

From Intermediate 20 and Intermediate 43

LCMS (System B, UV, ESI): Rₜ=1.10 min, [M+H]⁺ 602.3

### Intermediate 79 tert-butyl N-[(5-{4-[(3S)-3-ethylmorpholin-4-yl]-6-({[(4-methoxyphenyl)methyl]sulfamoyl}methyl)pyrimidin-2-yl}-1H-pyrrolo[3,2-b]pyridin-2-yl)methyl]-N-methylcarbamate

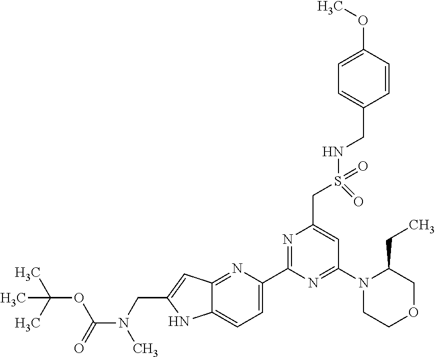 [SMILES: CC[C@H]1COCCN1c1cc(CS(=O)(=O)NCc2ccc(OC)cc2)nc(-c2ccc3[nH]c(CN(C)C(=O)OC(C)(C)C)cc3n2)n1]

From Intermediate 19 and Intermediate 65

LCMS (System A, UV, ESI): Rₜ=1.23 min, [M+H]⁺ 666.5

### Intermediate 80 1-{6-[(3S)-3-ethylmorpholin-4-yl]-2-[2-({[tris(propan-2-yl)silyl]oxy}methyl)-1H-pyrrolo[3,2-b]pyridin-5-yl]pyrimidin-4-yl}-N,N-dimethylmethanesulfonamide

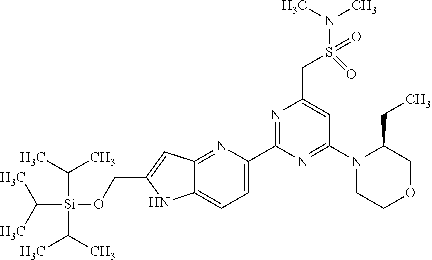 [SMILES: CC[C@H]1COCCN1c1cc(CS(=O)(=O)N(C)C)nc(-c2ccc3[nH]c(CO[Si](C(C)C)(C(C)C)C(C)C)cc3n2)n1]

From Intermediate 20 and Intermediate 64

LCMS (System A, UV, ESI): Rₜ=1.51 min, [M+H]⁺ 617.5

### Intermediate 81 1-{6-[(3S)-3-ethylmorpholin-4-yl]-2-[2-({[tris(propan-2-yl)silyl]oxy}methyl)-1H-pyrrolo[3,2-b]pyridin-5-yl]pyrimidin-4-yl}-N-[(4-methoxyphenyl)methyl]-N-methylmethanesulfonamide

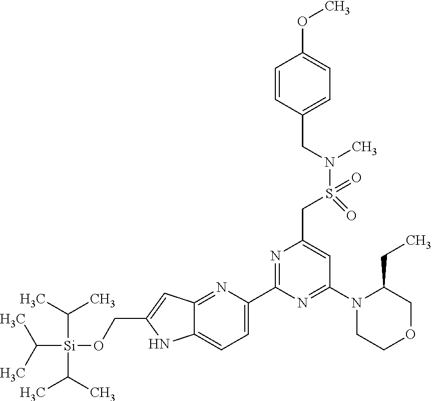 [SMILES: CC[C@H]1COCCN1c1cc(CS(=O)(=O)N(C)Cc2ccc(OC)cc2)nc(-c2ccc3[nH]c(CO[Si](C(C)C)(C(C)C)C(C)C)cc3n2)n1]

From Intermediate 20 and Intermediate 69

LCMS (System A, UV, ESI): Rₜ=1.63 min, [M+H]⁺ 723.4

### Intermediate 82 1-{6-{[(3S)-3-ethylmorpholin-4-yl]-2-(2-methyl-1H-pyrrolo[3,2-b]pyridin-5-yl}pyrimidin-4-yl}-N-[(4-methoxyphenyl)methyl]-N-methylmethanesulfonamide

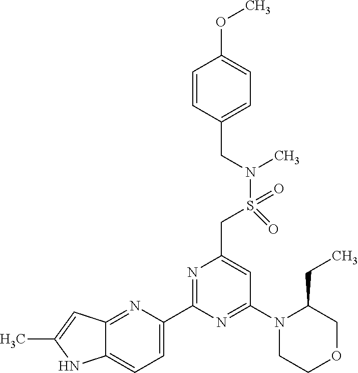 [SMILES: CC[C@H]1COCCN1c1cc(CS(=O)(=O)N(C)Cc2ccc(OC)cc2)nc(-c2ccc3[nH]c(C)cc3n2)n1]

From Intermediate 21 and Intermediate 69

LCMS (System A, UV, ESI): Rₜ=1.11 min [M+H]⁺ 551.2

### Intermediate 83 tert-butyl (S)-4-(2-(3-ethylmorpholino)-6-(2-methyl-1H-pyrrolo[3,2-b]pyridin-5-yl)pyridin-4-yl)-4-(methylsulfonyl)piperidine-1-carboxylate

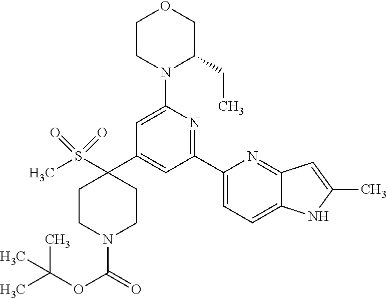 [SMILES: CC[C@H]1COCCN1c1cc(C2(S(C)(=O)=O)CCN(C(=O)OC(C)(C)C)CC2)cc(-c2ccc3[nH]c(C)cc3n2)n1]

A solution of tert-butyl (S)-4-(2-bromo-6-(3-ethylmorpholino)pyridin-4-yl)-4-(methylsulfonyl)piperidine-1-carboxylate (183 mg, 0.344 mmol), 2-methyl-1H-pyrrolo[3,2-b]pyridine-5-sulfinate, Sodium salt (160 mg, 0.733 mmol), K2CO3 (130 mg, 0.941 mmol), tricyclohexylphosphane (42 mg, 0.150 mmol) and palladium(II) acetate (16 mg, 0.071 mmol) in 1,4-Dioxane (3 mL) was placed in a microwave vial and sealed. The mixture was degassed using N2/vacuum 3 times. The mixture was heated to 150° C. for 20 h.

The reaction mixture was diluted with EtOAc (10 mL), filtered on a pre-packed celite cartridge then washed with EtOAc. The mixture was then partitioned with brine (40 mL). The organic layer was taken, passed through a hydrophobic frit, and concentrated under reduced pressure to give the desire product (139 mg).

Product was purified by reverse phase chromatography. The compound was dissolved in a minimum of DMSO/MeOH and eluted using 15-60% CH₃CN+0.1% formic acid/H2O+0.1% formic acid gradient and concentrated under reduced pressure to give tert-butyl (S)-4-(2-(3-ethylmorpholino)-6-(2-methyl-1H-pyrrolo[3,2-b]pyridin-5-yl)pyridin-4-yl)-4-(methylsulfonyl)piperidine-1-carboxylate (49 mg, 0.084 mmol, 24.42% yield) as a yellow solid

LCMS (System B, UV, ESI): Rₜ=0.80 min, [M+H]⁺ 584

### Intermediate 84 (S)-3-ethyl-4-(6-(2-methyl-1-(phenylsulfonyl)-1H-pyrrolo[3,2-b]pyridin-5-yl)-4-((methylsulfonyl)methyl) pyridin-2-yl)morpholine

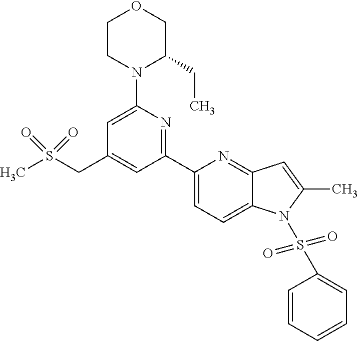 [SMILES: CC[C@H]1COCCN1c1cc(CS(C)(=O)=O)cc(-c2ccc3c(cc(C)n3S(=O)(=O)c3ccccc3)n2)n1]

To a stirred solution of sodium (S)-6-(3-ethylmorpholino)-4-((methyl sulfonyl)methyl) pyridine-2-sulfinate (100 mg, 0.271 mmol), potassium carbonate (57.7 mg, 0.417 mmol), tricyclohexylphosphine (11.70 mg, 0.042 mmol) and palladium(II) acetate (4.68 mg, 0.021 mmol) in anhydrous 1,4-Dioxane (1.2 mL), was added 5-chloro-2-methyl-1-(phenylsulfonyl)-1H-pyrrolo[3,2-b]pyridine (64 mg, 0.209 mmol). The reaction vessel was evacuated and purged with nitrogen (×3), before the mixture was stirred at 150° C. for 20 h. Potassium carbonate (29 mg, 0.21 mmol) and palladium(II) acetate (2 mg, 0.01 mmol) were added. The reaction vessel was evacuated and purged with nitrogen (×3), and the mixture was stirred at 150° C. for 2.5 h. Potassium carbonate (15 mg, 0.10 mmol) and palladium(II) acetate (2 mg, 0.01 mmol) were added. The reaction vessel was evacuated and purged with nitrogen (×3), and the mixture was stirred at 150° C. for 16 h.

The reaction mixture was diluted with ethyl acetate (20 mL) and filtered over celite. The celite cartridge was washed with ethyl acetate (100 mL) and the filtrate washed with water (25 mL). The aqueous phase was extracted with ethyl acetate (2×25 mL), and the combined organic phase was passed through a hydrophobic frit and evaporated in vacuo, to afford a yellow solid.

The solid was dissolved in 1:1 DMSO:MeOH (2.0 mL) and aliquots (2×1.0 mL) were purified by Mass Directed AutoPreparative HPLC (MDAP) on OA MDAP (Xselect CSH column 150 mm×30 mm i.d. 5 μM packing diameter at ambient temperature) eluting with solvents A/B (A: 10 mM ammonium bicarbonate in water adjusted to pH 10 with ammonia solution, B: acetonitrile) using method C runs. The product-containing fractions were combined and evaporated in vacuo to afford (S)-3-ethyl-4-(6-(2-methyl-1-(phenylsulfonyl)-1H-pyrrolo[3,2-b]pyridin-5-yl)-4-((methylsulfonyl)methyl) pyridin-2-yl)morpholine (32 mg, 0.058 mmol, 27.7% yield), as a white solid.

LCMS (SystemA, UV, ESI): Rₜ=1.29 min, [M+H]⁺ 555.

### Intermediate 85 1 (S)-1-(2-(3-ethylmorpholino)-6-(2-methyl-1H-pyrrolo[3,2-b]pyridin-5-yl)pyridin-4-yl)-N-(4-methoxybenzyl)methanesulfonamide

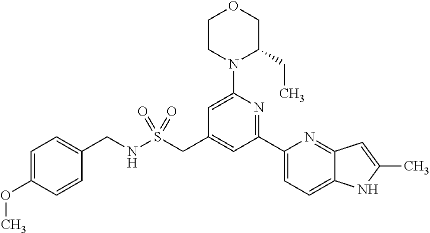 [SMILES: CC[C@H]1COCCN1c1cc(CS(=O)(=O)NCc2ccc(OC)cc2)cc(-c2ccc3[nH]c(C)cc3n2)n1]

A solution of (S)-1-(2-bromo-6-(3-ethylmorpholino)pyridin-4-yl)-N-(4-methoxybenzyl) methanesulfonamide (150 mg, 0.310 mmol), sodium 2-methyl-1H-pyrrolo[3,2-b]pyridine-5-sulfinate (85 mg, 0.390 mmol), K2CO3 (82 mg, 0.593 mmol), palladium(II) acetate (11 mg, 0.049 mmol) and P(tBu)2Me.HBF4 (19 mg, 0.077 mmol) in 1,4-Dioxane (3000 μl) was degassed under a stream of nitrogen for 10 minutes. The reaction mixture was then sealed, heated to 150° C. and stirred at 150° C. for 16 h. LCMS

The reaction mixture was concentrated under a stream of nitrogen, partitioned between 4.5 mL 0.5 M EDTA solution and 4.5 mL EtOAc. The organic layer was combined, dried over a hydrophobic frit, and concentrated under reduced pressure. The residue was dissolved in 1 mL DCM and eluted on a 12 g Silica gel column in cyclohexane with a gradient of 0-100% EtOAc.

The desired fractions were concentrated under reduced pressure to give (S)-1-(2-(3-ethylmorpholino)-6-(2-methyl-1H-pyrrolo[3,2-b]pyridin-5-yl)pyridin-4-yl)-N-(4-methoxybenzyl) methanesulfonamide (107 mg, 0.180 mmol, 58.1% yield) as a yellow gum.

LCMS (System A, UV, ESI): Rₜ=1.17 min, [M+H]⁺ 536.2.

Similarly prepared were:

### Intermediate 86 (S)-5-ethyl-4-(4-(4-(methylsulfonyl)tetrahydro-2H-pyran-4-yl)-6-(2-(((triisopropylsilyloxy)methyl)-1H-pyrrolo[3,2-b]pyridin-5-yl)pyridin-2-yl)morpholin-3-one

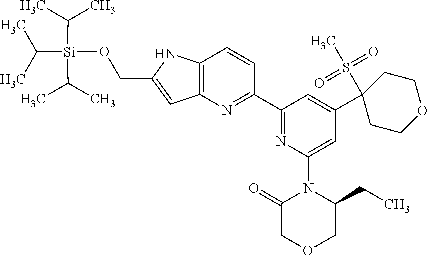 [SMILES: CC[C@H]1COCC(=O)N1c1cc(C2(S(C)(=O)=O)CCOCC2)cc(-c2ccc3[nH]c(CO[Si](C(C)C)(C(C)C)C(C)C)cc3n2)n1]

From Intermediate 48 and Intermediate 20

LCMS (System A, UV, ESI): Rₜ=1.51 min, [M+H]⁺ 671

### Intermediate 87 (S)-5-ethyl-4-(4-((methylsulfonyl)methyl)-6-(2-(((triisopropylsilyloxy)methyl)-1H-pyrrolo[3,2-b]pyridin-5-yl)pyridin-2-yl)morpholin-3-one

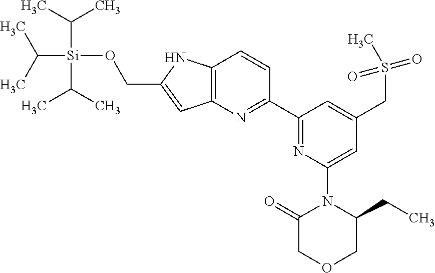 [SMILES: CC[C@H]1COCC(=O)N1c1cc(CS(C)(=O)=O)cc(-c2ccc3[nH]c(CO[Si](C(C)C)(C(C)C)C(C)C)cc3n2)n1]

From Intermediate 47 and Intermediate 20

LCMS (System B, UV, ESI): Rₜ=1.17 min, [M+H]⁺ 601

### Intermediate 88 (S)-1-(2-(3-ethyl-5-oxomorpholino)-6-(2-(((triisopropylsilyl)oxy) methyl)-1H-pyrrolo[3,2-b]pyridin-5-yl)pyridin-4-yl)-N,N-dimethylmethanesulfonamide

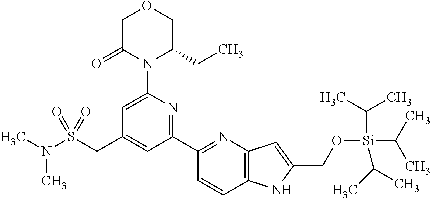 [SMILES: CC[C@H]1COCC(=O)N1c1cc(CS(=O)(=O)N(C)C)cc(-c2ccc3[nH]c(CO[Si](C(C)C)(C(C)C)C(C)C)cc3n2)n1]

From Intermediate 96 and Intermediate 20

LCMS (System A, UV, ESI): Rₜ=1.57 min, [M+H]⁺ 630.4

### Intermediate 89 tert-butyl (S)-((5-(4-((N,N-dimethylsulfamoyl)methyl)-6-(3-ethyl-5-oxomorpholino)pyridin-2-yl)-1H-pyrrolo[3,2-b]pyridin-2-yl)methyl)(methyl)carbamate

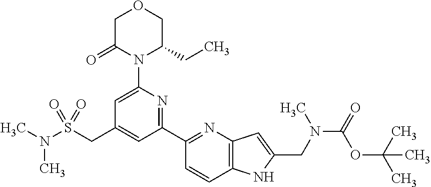 [SMILES: CC[C@H]1COCC(=O)N1c1cc(CS(=O)(=O)N(C)C)cc(-c2ccc3[nH]c(CN(C)C(=O)OC(C)(C)C)cc3n2)n1]

From Intermediate 96 and Intermediate 19

LCMS (System B, UV, ESI): Rₜ=0.78 min, [M+H]⁺ 587.4

### Intermediate 90 (2R,3R)-4-(6-bromo-4-((methylsulfonyl)methyl)pyridin-2-yl)-2,3-dimethylmorpholine

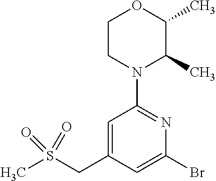 [SMILES: C[C@@H]1[C@@H](C)OCCN1c1cc(CS(C)(=O)=O)cc(Br)n1]

2,2,6,6-Tetramethylpiperidine (1.6 mL, 9.37 mmol) was added to 2,6-dibromo-4-(methylsulfonyl) methyl)pyridine (200 mg, 0.608 mmol) and (2R,3R)-2,3-dimethylmorpholine hydrochloride (80 mg, 0.528 mmol). The reaction mixture was degassed under a flow of N2 gas for 5 minutes, before being sealed and heated to 150° C. for 16 hours.

Further (2R,3R)-2,3-dimethylmorpholine hydrochloride (46.1 mg, 0.304 mmol) was added to the reaction mixture which was left to stir at 150° C. for 24 hours.

The reaction mixture was cooled and quenched with saturated aqueous ammonium chloride (10 mL), water (5 mL) and extracted with EtOAc (2×20 mL). The organic layers were combined and washed with water (10 mL) and brine (10 mL), before being filtered through a hydrophobic frit and concentrated in vacuo. The crude product was purified using a 24 g silica flash column, eluting with EtOAc:Cyclohexane (40-80%) for 20 minutes. The relevant fractions were concentrated in vacuo to give (2R,3R)-4-(6-bromo-4-((methylsulfonyl)methyl) pyridin-2-yl)-2,3-dimethylmorpholine (169 mg, 0.465 mmol, 77% yield) as an orange gum.

LCMS (System A, UV, ESI): Rₜ=0.99 min, [M+H]⁺ 365.1

### Intermediate 91 tert-butyl ((5-(6-((2R,3R)-2,3-dimethylmorpholino)-4-((methylsulfonyl)methyl)pyridin-2-yl)-1Hpyrrolo[3,2-b]pyridin-2-yl)methyl)(methyl)carbamate

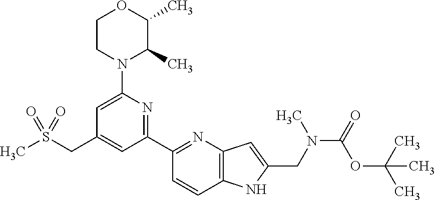 [SMILES: C[C@@H]1[C@@H](C)OCCN1c1cc(CS(C)(=O)=O)cc(-c2ccc3[nH]c(CN(C)C(=O)OC(C)(C)C)cc3n2)n1]

From Intermediate 90 and Intermediate 19

1H NMR (400 MHz, CHLOROFORM-d) δ 9.17 (br. s., 1H), 8.27 (d, J=8.6 Hz, 1H), 7.82 (s, 1H), 7.72 (d, J=8.6 Hz, 1H), 6.72 (s, 1H), 6.65 (s, 1H), 4.51 (br. s., 2H), 4.27 (s, 2H), 4.24-4.11 (m, 2H), 4.09-3.98 (m, 1H), 3.96-3.89 (m, 1H), 3.37 (dt, J=4.4, 12.3 Hz, 1H), 2.95 (br. s., 1H), 2.87 (s, 3H), 1.54 (s, 9H), 1.44 (d, J=6.6 Hz, 3H), 1.35 (d, J=6.6 Hz, 3H)

LCMS (System A, UV, ESI): Rₜ=1.13 min, [M+H]⁺ 544.3

### Intermediate 97 Tert-butyl ((5-chloro-1-((2-(trimethylsilyl)ethoxy)methyl)-1H-pyrrolo[3,2-b]pyridin-2-yl)methyl)(methyl)carbamate

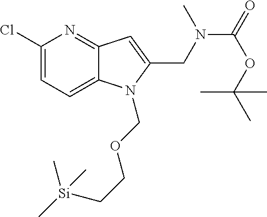 [SMILES: CN(Cc1cc2nc(Cl)ccc2n1COCC[Si](C)(C)C)C(=O)OC(C)(C)C]

Sodium hydride (0.352 g, 8.79 mmol) was added to a solution of tert-butyl ((5-chloro-1H-pyrrolo[3,2-b]pyridin-2-yl)methyl)(methyl)carbamate (2 g, 6.76 mmol) in DMF (10 ml) at 0° C. and the mixture was stirred for 10 min, then SEM-Cl (1.439 mL, 8.11 mmol) was added and the mixture was stirred for a further 2 h. The mixture was diluted with water (40 ml) and extracted with EtOAc (2×40 ml). The combined organics were washed with water and brine (50 ml each), dried and evaporated in vacuo to give a brown gum. This was dissolved in DCM and loaded onto a 50 g silica column, then eluted with 0-50% EtOAc/cyclohexane and product-containing fractions evaporated in vacuo to give tert-butyl ((5-chloro-1-((2-(trimethylsilyl)ethoxy)methyl)-1H-pyrrolo[3,2-b]pyridin-2-yl)methyl)(methyl)-carbamate (2.1 g, 4.93 mmol, 72.9% yield) as a pale yellow gum. LCMS (System A, UV, ESI): Rₜ=1.54 min, [M+H]⁺ 426

### Intermediate 98 tert-butyl ((5-cyano-1-((2-(trimethylsilyl)ethoxy)methyl)-1H-pyrrolo[3,2-b]pyridin-2-yl)methyl)(methyl)carbamate

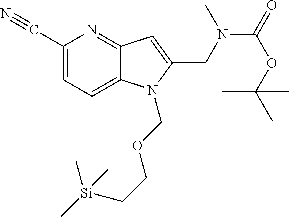 [SMILES: CN(Cc1cc2nc(C#N)ccc2n1COCC[Si](C)(C)C)C(=O)OC(C)(C)C]

A round bottom flask was charged with potassium acetate (0.059 g, 0.601 mmol), potassium hexacyanoferrate(II) (1.016 g, 2.406 mmol), tert-butyl ((5-chloro-1-((2-(trimethylsilyl)ethoxy)methyl)-1H-pyrrolo[3,2-b]pyridin-2-yl)methyl)(methyl)carbamate (2.05 g, 4.81 mmol), 2G [Pd] XPhos (0.189 g, 0.241 mmol), and XPhos (0.115 g, 0.241 mmol) before the flask was fitted with a reflux condenser and flushed with nitrogen. Water (20.00 ml) and 1,4-Dioxane (20 ml) were added to the reaction and the resulting suspension degassed under a strong pressure of nitrogen with vigorous stirring. The reaction was then heated to 100° C. for 24 hours before cooling to room temperature.

The mixture was diluted with water (50 ml) and extracted with EtOAc (2×50 ml), then the organics dried and evaporated in vacuo to give a brown gum. This was purified by chromatography on a 50 g silica column eluting with 0-50% EtOAc/cyclohexane and product-containing fractions evaporated in vacuo to give tert-butyl ((5-cyano-1-((2-(trimethylsilyl)-ethoxy)methyl)-1H-pyrrolo[3,2-b]pyridin-2-yl)methyl)(methyl)carbamate (1.91 g, 4.58 mmol, 95% yield) as a colourless gum. LCMS (System A, UV, ESI): Rₜ=1.46 min, [M+H]⁺ 417

Similarly prepared was:

### Intermediate 99 tert-butyl ((5-cyano-1H-pyrrolo[3,2-b]pyridin-2-yl)methyl)(methyl)carbamate

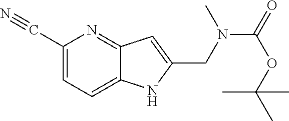 [SMILES: CN(Cc1cc2nc(C#N)ccc2[nH]1)C(=O)OC(C)(C)C]

LCMS (System A, UV, ESI): Rₜ=1.00 min, [M+H]⁺ 287

### Intermediate 100 tert-butyl ((5-(N-hydroxycarbamimidoyl)-1-((2-(trimethylsilylethoxy)methyl)-1H-pyrrolo[3,2-b]pyridin-2-yl)methyl)(methyl)carbamate

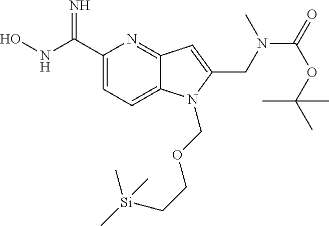 [SMILES: CN(Cc1cc2nc(C(=N)NO)ccc2n1COCC[Si](C)(C)C)C(=O)OC(C)(C)C]

tert-butyl ((5-cyano-1-((2-(trimethylsilyl)ethoxy)methyl)-1H-pyrrolo[3,2-b]pyridin-2-yl)methyl)(methyl)carbamate (1900 mg, 4.56 mmol), hydroxylamine hydrochloride (333 mg, 4.79 mmol) and triethylamine (1271 μl, 9.12 mmol) were heated at 70° C. for 2 h in Ethanol (10000 μl) then the mixture was evaporated in vacuo and partitioned between water (30 ml) and EtOAc (50 ml). The organic layer was dried and evaporated in vacuo to give tert-butyl ((5-(N-hydroxycarbamimidoyl)-1-((2-(trimethylsilyl)-ethoxy)methyl)-1H-pyrrolo[3,2-b]pyridin-2-yl)methyl)(methyl)carbamate (1.71 g, 3.80 mmol, 83% yield) as a colourless solid. LCMS (System A, UV, ESI): Rₜ=1.31 min, [M+H]⁺ 450

### Intermediate 101 tert-butyl ((5-carbamimidoyl-1-((2-(trimethylsilylethoxy)methyl)-1H-pyrrolo[3,2-b]pyridin-2-yl)methyl)(methyl)carbamate

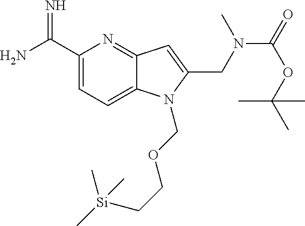 [SMILES: CN(Cc1cc2nc(C(=N)N)ccc2n1COCC[Si](C)(C)C)C(=O)OC(C)(C)C]

tert-butyl ((5-(N-hydroxycarbamimidoyl)-1-((2-(trimethylsilyl)ethoxy)methyl)-1H-pyrrolo[3,2-b]pyridin-2-yl)methyl)(methyl)carbamate (1.65 g, 3.67 mmol) was suspended in Methanol (18.35 ml) and acetic anhydride (0.7 ml, 7.42 mmol) was added. The flask was purged with nitrogen then Pd—C (0.391 g, 0.367 mmol) and ammonium formate (1.2 g, 19.03 mmol) were added and the mixture heated at reflux for 2 h.

The mixture was filtered through Celite and evaporated in vacuo to give a colourless solid, containing residual ammonium formate. The crude was partitioned between water and DCM and the organic layer dried and evaporated in vacuo to give tert-butyl ((5-carbamimidoyl-1-((2-(trimethylsilyl)ethoxy)methyl)-1H-pyrrolo[3,2-b]pyridin-2-yl)methyl)(methyl)carbamate (1.52 g, 3.51 mmol, 96% yield) as a colourless solid. LCMS (System B, UV, ESI): Rₜ=0.88 min, [M+H]⁺ 434 Similarly prepared was:

### Intermediate 102 tert-butyl ((5-carbamimidoyl-1H-pyrrolo[3,2-b]pyridin-2-yl)methyl)-(methyl)carbamate acetate

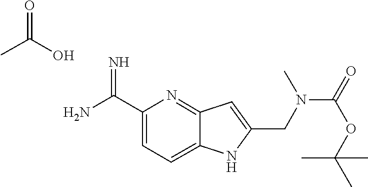

To a solution of tert-butyl ((5-cyano-1H-pyrrolo[3,2-b]pyridin-2-yl)methyl)(methyl)carbamate (7.68 g, 26.8 mmol) and hydroxylamine hydrochloride (1.957 g, 28.2 mmol) and Methanol (60 mL) in a sealed oven-dried microwave vial was added triethylamine (7.48 mL, 53.6 mmol) and the reaction mixture heated to 70° C. for 2 hours. The solvent was removed under a flow of nitrogen and sat NH₄Cl solution (100 ml) and EtOAc (100 ml) was added. The organic layer was separated and the aqueous layer extracted with EtOAc (3×100 ml) before the combined organic layers were passed through a hydrophobic frit and concentrated in vacuo.

The crude hydroxyamidine intermediate was then taken up in Methanol (60 mL) and cooled to 0° C. before the addition of acetic anhydride (5.06 mL, 53.6 mmol). The reaction was then allowed to warm to room temperature where it was stirred for 30 mins. The solution was transferred to a hydrogenation flask charged with Pd—C(2.85 g, 2.68 mmol) and placed under a hydrogen atmosphere where it was stirred for 2 hours at room temperature. After this time the hydrogen atmosphere was removed and the solvent removed in vacuo and the resulting crude product was washed with TBME to give tert-butyl ((5-carbamimidoyl-1H-pyrrolo[3,2-b]pyridin-2-yl)methyl)(methyl)carbamate acetate (8.40 g, 23.11 mmol, 86% yield) as a white solid. LCMS (System A, UV, ESI): Rₜ=0.56 min, [M+H]⁺ 304

### Intermediate 103 tert-butyl ((5-(4-(chloromethyl)-6-hydroxypyrimidin-2-yl)-1-((2-(trimethylsilylethoxy)methyl)-1H-pyrrolo[3,2-b]pyridin-2-yl)methyl)(methyl)carbamate

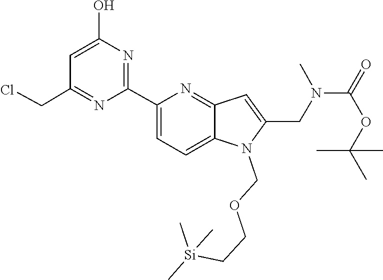 [SMILES: CN(Cc1cc2nc(-c3nc(O)cc(CCl)n3)ccc2n1COCC[Si](C)(C)C)C(=O)OC(C)(C)C]

To a solution of ethyl 4-chloro-3-oxobutanoate (0.623 ml, 4.61 mmol) and tert-butyl ((5-carbamimidoyl-1-((2-(trimethylsilyl)ethoxy)methyl)-1H-pyrrolo[3,2-b]pyridin-2-yl)methyl)-(methyl)carbamate (1000 mg, 2.306 mmol) in methanol (5 ml) under a nitrogen atmosphere was added DBU (0.869 ml, 5.77 mmol) and the reaction stirred at 65° C. for 1 hour. The reaction was concentrated in vacuo and the residue partitioned between EtOAc (20 ml) and ammonium chloride solution (20 ml). The organic phase was dried and evaporated in vacuo to give a pale yellow gum. The crude product was purified by flash column chromatography on a 50 g silica column (0-100% EtOAc (1% AcOH) in Cyclohexane) to give tert-butyl ((5-(4-(chloromethyl)-6-hydroxypyrimidin-2-yl)-1-((2-trimethylsilyl)ethoxy)-methyl)-1H-pyrrolo[3,2-b]pyridin-2-yl)methyl)(methyl)carbamate (1.2 g, 2.247 mmol, 97% yield) as a pale yellow solid. LCMS (System B, UV, ESI): Rₜ=1.48 min, [M+H]⁺ 534

Similarly prepared were:

### Intermediate 104 tert-butyl ((5-(4-(chloromethyl)-6-hydroxypyrimidin-2-yl)-1H-pyrrolo[3,2-b]pyridin-2-ylmethyl(methylcarbamate

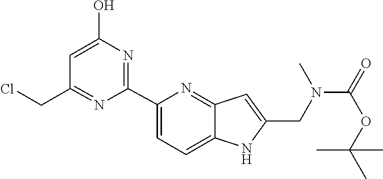 [SMILES: CN(Cc1cc2nc(-c3nc(O)cc(CCl)n3)ccc2[nH]1)C(=O)OC(C)(C)C]

LCMS (SystemA, UV, ESI): Rₜ=1.04 min, [M+H]⁺ 404

### Intermediate 105 2-(2-(((tert-butoxycarbonyl)(methyl)amino)methyl)-1-((2-(trimethylsilylethoxy)methyl)-1H-pyrrolo[3,2-b]pyridin-5-yl)-6-(chloromethyl)pyrimidin-4-yl trifluoromethanesulfonate

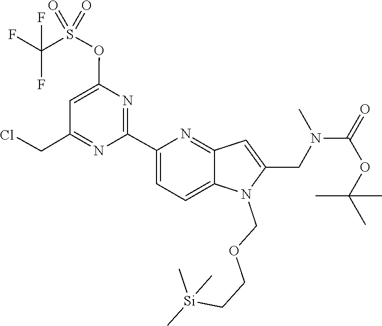 [SMILES: CN(Cc1cc2nc(-c3nc(CCl)cc(OS(=O)(=O)C(F)(F)F)n3)ccc2n1COCC[Si](C)(C)C)C(=O)OC(C)(C)C]

Trifluoromethanesulfonic anhydride (45.5 μl, 0.270 mmol) was added to a solution of tert-butyl ((5-(4-(chloromethyl)-6-hydroxypyrimidin-2-yl)-1-((2-(trimethylsilyl)ethoxy)methyl)-1H-pyrrolo[3,2-b]pyridin-2-yl)methyl)(methyl)carbamate (120 mg, 0.225 mmol) and N-ethyl-N-isopropylpropan-2-amine (500 μl, 2.86 mmol) in DCM (5 ml) at 0° C. and the mixture was stirred for 30 minutes. The mixture was washed with water, then the solvent dried and evaporated in vacuo to give 2-(2-(((tert-butoxycarbonyl)(methyl)amino)-methyl)-1-((2-(trimethylsilyl)ethoxy)methyl)-1H-pyrrolo[3,2-b]pyridin-5-yl)-6-(chloromethyl)-pyrimidin-4-yl trifluoromethanesulfonate (155 mg, 0.233 mmol, 104% yield) as a yellow gum. LCMS (System B, UV, ESI): Rₜ=1.58 min, [M+H]⁺ 666

### Intermediate 106 tert-butyl (S)-((5-(4-(chloromethyl)-6-(3-ethylmorpholino)pyrimidin-2-yl)-1-((2-(trimethylsilyl)ethoxy)methyl)-1H-pyrrolo[3,2-b]pyridin-2-yl)methyl)(methyl)carbamate

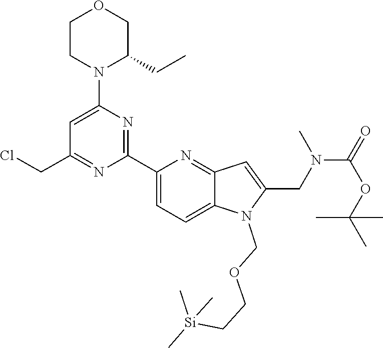 [SMILES: CC[C@H]1COCCN1c1cc(CCl)nc(-c2ccc3c(cc(CN(C)C(=O)OC(C)(C)C)n3COCC[Si](C)(C)C)n2)n1]

2-(2-(((tert-butoxycarbonyl)(methyl)amino)methyl)-1-((2-(trimethylsilyl)ethoxy)methyl)-1H-pyrrolo[3,2-b]pyridin-5-yl)-6-(chloromethyl)pyrimidin-4-yl trifluoromethanesulfonate (150 mg, 0.225 mmol), (S)-3-ethylmorpholine, Hydrochloride (41.0 mg, 0.270 mmol) and DIPEA (0.079 ml, 0.450 mmol) were heated in Acetonitrile (10 ml) at 50° C. for 2 h, then allowed to stand at room temperature over the weekend. The mixture was diluted with water and extracted with EtOAc and the solvent dried and evaporated in vacuo to give a pale yellow gum. The crude was dissolved in DCM and loaded onto a 25 g silica column, then eluted with 0-100% (25% EtOH/EtOAc 1% NH₄₀H)/cyclohexane and product-containing fractions evaporated in vacuo to give tert-butyl (S)-((5-(4-(chloromethyl)-6-(3-ethylmorpholino)-pyrimidin-2-yl)-1-((2-(trimethylsilyl)ethoxy)methyl)-1H-pyrrolo[3,2-b]pyridin-2-yl)methyl)-(methyl)carbamate (94 mg, 0.149 mmol, 66.1% yield) as a pale yellow gum. LCMS (System B, UV, ESI): Rₜ=1.37 min, [M+H]⁺ 631

### Intermediate 107 sodium propane-1-sulfinate

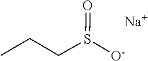

To a solution of sodium sulfite (2.395 g, 19.00 mmol) in Water (10 mL) was added (portionwise) propane-1-sulfonyl chloride (0.789 mL, 7.01 mmol) and solid sodium carbonate (1.591 g, 15.01 mmol). The reaction mixture was heated to reflux for 1 hr then cooled to room temperature. The mixture was concentrated to dryness, then slurried in absolute ethanol. The slurry was heated to reflux for 1 hr, after which it was cooled to room temperature, filtered and concentrated to afford sodium propane-1-sulfinate (885 mg, 6.80 mmol, 97% yield). ¹H NMR (400 MHz, METHANOL-d₄) δ ppm 1.03 (t, J=7.58 Hz, 3H) 1.59-1.70 (m, 2H) 2.23-2.30 (m, 2H)

### Intermediate 108 tert-butyl (S)-((5-(4-(3-ethylmorpholino)-6-((ethylsulfonyl)methyl)-pyrimidin-2-yl)-1-((2-(trimethylsilyl)ethoxy)methyl)-1H-pyrrolo[3,2-b]pyridin-2-yl)methyl)-(methyl)carbamate

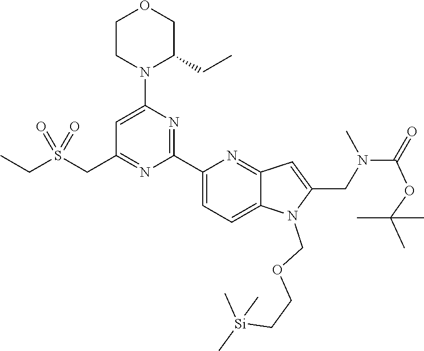 [SMILES: CC[C@H]1COCCN1c1cc(CS(=O)(=O)CC)nc(-c2ccc3c(cc(CN(C)C(=O)OC(C)(C)C)n3COCC[Si](C)(C)C)n2)n1]

Sodium ethanesulfinate (24.83 mg, 0.214 mmol) was added to a solution of tert-butyl (S)-((5-(4-(chloromethyl)-6-(3-ethylmorpholino)pyrimidin-2-yl)-1-((2-(trimethylsilyl)ethoxy)methyl)-1H-pyrrolo[3,2-b]pyridin-2-yl)methyl)(methyl)carbamate (90 mg, 0.143 mmol) and potassium iodide (5 mg, 0.030 mmol) in Methanol (5 mL) at room temperature and the mixture was heated at 60 C for 2 h, then cooled, evaporated in vacuo and the residue partitioned between DCM (10 ml) and water (10 ml). The organic layer was dried and evaporated in vacuo to give tert-butyl (S)-((5-(4-(3-ethylmorpholino)-6-((ethylsulfonyl)methyl)pyrimidin-2-yl)-1-((2-(trimethylsilyl)ethoxy)methyl)-1H-pyrrolo[3,2-b]pyridin-2-yl) methyl)(methyl)carbamate (62 mg, 0.090 mmol, 63.1% yield) as a brown solid which was used crude in the next step.

LCMS (System B, UV, ESI): Rₜ=1.05 min, [M+H]⁺ 689

Similarly prepared were:

### Intermediate 109 tert-butyl (S)-((5-(4-(3-ethylmorpholino)-6-((isopropylsulfonyl)methyl)-pyrimidin-2-yl)-1-((2-(trimethylsilyl)ethoxy)methyl)-1H-pyrrolo[3,2-b]pyridin-2-yl)methyl)-(methyl)carbamate

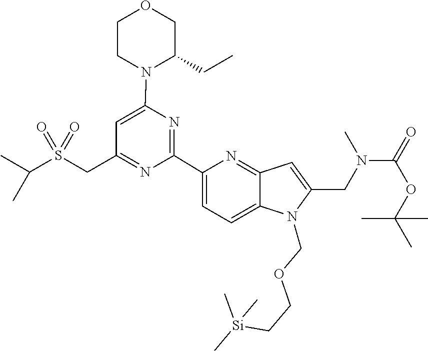 [SMILES: CC[C@H]1COCCN1c1cc(CS(=O)(=O)C(C)C)nc(-c2ccc3c(cc(CN(C)C(=O)OC(C)(C)C)n3COCC[Si](C)(C)C)n2)n1]

LCMS (System B, UV, ESI): Rₜ=1.12 min, [M+H]⁺ 703 Intermediate 110 tert-butyl (S)-((5-(4-(3-ethylmorpholino)-6-((propylsulfonyl)-methyl)pyrimidin-2-yl)-1-((2-(trimethylsilyl)ethoxy)methyl)-1H-pyrrolo[3,2-b]pyridin-2-yl)methyl)(methyl)-carbamate

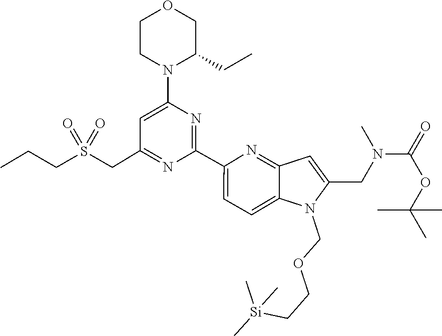 [SMILES: CCCS(=O)(=O)Cc1cc(N2CCOC[C@@H]2CC)nc(-c2ccc3c(cc(CN(C)C(=O)OC(C)(C)C)n3COCC[Si](C)(C)C)n2)n1]

From Intermediate 98 and Intermediate 100

LCMS (System B, UV, ESI): Rₜ=1.13 min, [M+H]⁺ 703

### Intermediate 111 sodium (2-(2-(((tert-butoxycarbonyl)(methyl)amino)methyl)-1H-pyrrolo[3,2-b]pyridin-5-yl)-6-hydroxypyrimidin-4-yl)methanesulfinate

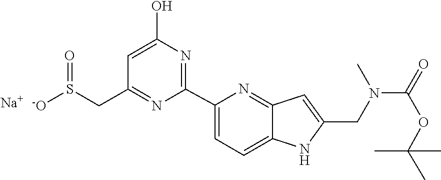

To a solution of tert-butyl ((5-(4-(chloromethyl)-6-hydroxypyrimidin-2-yl)-1H-pyrrolo[3,2-b]pyridin-2-yl)methyl)(methyl)carbamate (1350 mg, 3.34 mmol) in Dimethyl Sulfoxide (DMSO) (16000 μl) at room temperature was added sodium 3-methoxy-3-oxopropane-1-sulfinate (640 mg, 3.68 mmol) and the reaction was stirred at room temperature for 24 hours. Water (100 ml) was added to the reaction and the precipitate formed was filtered off and dried in vacuo to give sodium (2-(2-(((tert-butoxycarbonyl)(methyl)amino)methyl)-1H-pyrrolo[3,2-b]pyridin-5-yl)-6-hydroxypyrimidin-4-yl)methanesulfinate (1.13 g, 2.233 mmol, 66.8% yield) a pale brown solid.

The crude mixture was then dissolved in Tetrahydrofuran (THF) (12000 μl) and Methanol (4000 μl) before the dropwise addition of sodium methoxide (1055 μl, 6.69 mmol) solution. The resulting reaction mixture was left to stir at room temperature for 2 hours. The reaction solvent was removed in vacuo to give sodium (2-(2-(((tert-butoxycarbonyl)(methyl)amino)methyl)-1H-pyrrolo[3,2-b]pyridin-5-yl)-6-hydroxypyrimidin-4-yl)methanesulfinate (1.13 g, 2.233 mmol, 66.8% yield) as a brown solid. ¹H NMR (400 MHz, DMSO-d₆) δ ppm 1.38-1.54 (m, 9H) 2.88 (s, 2H) 4.57 (s, 2H) 5.61 (s, 1H) 6.64 (s, 1H) 7.73 (br d, J=8.56 Hz, 1H) 8.19 (d, J=8.56 Hz, 1H) 11.19-11.82 (m, 1H).

### Intermediate 112 tert-butyl ((5-(4-((N,N-dimethylsulfamoyl)methyl)-6-hydroxypyrimidin-2-yl)-1H-pyrrolo[3,2-b]pyridin-2-yl)methyl)(methyl)carbamate

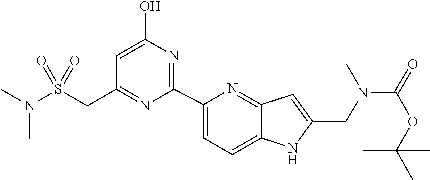 [SMILES: CN(Cc1cc2nc(-c3nc(O)cc(CS(=O)(=O)N(C)C)n3)ccc2[nH]1)C(=O)OC(C)(C)C]

To a solution of NCS (40.9 mg, 0.306 mmol) in solvent at room temperature was added dimethylamine (345 μl, 0.689 mmol) solution dropwise and the reaction allowed to stir for 15 mins. After this time the solution was added to a separate vial containing sodium (2-(2-(((tert-butoxycarbonyl)(methyl)amino)methyl)-1H-pyrrolo[3,2-b]pyridin-5-yl)-6-hydroxypyrimidin-4-yl)methanesulfinate (75 mg, 0.153 mmol) and the reaction stirred for a further 2 hours at room temperature.

The reaction was quenched by the addition of sat NH₄Cl solution (20 ml, aq) and EtOAc. The organic layer was separated and the aqueous layer extracted with EtOAc (3×20 ml) before the combined layers were washed with water (20 ml), 5% LiCl solution (20 ml), passed through a hydrophobic frit before concentrating in vaccuo to give tert-butyl ((5-(4-((N,N-dimethylsulfamoyl) methyl)-6-hydroxypyrimidin-2-yl)-1H-pyrrolo[3,2-b]pyridin-2-yl)methyl)(methyl)carbamate (49 mg, 0.090 mmol, 59.1% yield) as a brown solid. LCMS (System A, UV, ESI): Rₜ=0.91 min, [M+H]⁺ 477.

Similarly prepared was:

### Intermediate 113 tert-butyl (S)-((5-(4-((N,N-diethylsulfamoyl)methyl)-6-(3-ethylmorpholino)pyrimidin-2-yl)-1H-pyrrolo[3,2-b]pyridin-2-yl)methyl)(methyl)carbamate

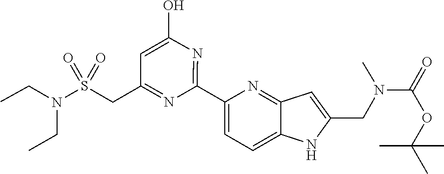 [SMILES: CCN(CC)S(=O)(=O)Cc1cc(O)nc(-c2ccc3[nH]c(CN(C)C(=O)OC(C)(C)C)cc3n2)n1]

LCMS (System A, UV, ESI): Rₜ=1.04 min, [M+H]⁺ 505.

### Intermediate 114 tert-butyl (S)-((5-(4-((N,N-dimethylsulfamoyl)methyl)-6-(3-ethylmorpholino)pyrimidin-2-yl)-1H-pyrrolo[3,2-b]pyridin-2-yl)methyl)(methyl)carbamate

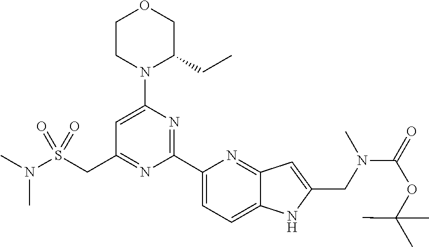 [SMILES: CC[C@H]1COCCN1c1cc(CS(=O)(=O)N(C)C)nc(-c2ccc3[nH]c(CN(C)C(=O)OC(C)(C)C)cc3n2)n1]

To a solution of tert-butyl ((5-(4-((N,N-dimethylsulfamoyl)methyl)-6-hydroxypyrimidin-2-yl)-1H-pyrrolo[3,2-b]pyridin-2-yl)methyl)(methyl)carbamate (49 mg, 0.090 mmol) and BOP (52.0 mg, 0.118 mmol) in Dimethyl Sulfoxide (DMSO) (226 μl) and N,N-Dimethylformamide (DMF) (226 μl) at room temperature was added DIPEA (79 μl, 0.452 mmol) and the reaction stirred for 1 hour at this temperature. After this time (S)-3-ethylmorpholine hydrochloride (24.70 mg, 0.163 mmol) was added in one portion and the reaction mixture was heated to 60° C. for 18 hours. After this time the reaction was quenched by the addition of sat aq NH₄Cl and diluted with EtOAc. The organic layer was separated and the aqueous extracted with EtOAc (3×10 ml) before the combined organic phases were washed with water (10 ml), passed through a hydrophobic frit and concentrated in vaccuo.

The crude residue was purified by flash column chromatography (0-80% EtOAc in cyclohexane) to give tert-butyl (S)-((5-(4-((N,N-dimethylsulfamoyl)methyl)-6-(3-ethylmorpholino)pyrimidin-2-yl)-1H-pyrrolo[3,2-b]pyridin-2-yl)methyl)(methyl)carbamate (23 mg, 0.040 mmol, 44.3% yield) as a brown solid. ¹H NMR (400 MHz, CHLOROFORM-d) δ ppm 0.99 (br t, J=7.46 Hz, 3H) 1.53 (s, 9H) 1.78-1.99 (m, 2H) 2.86 (s, 6H) 2.92 (s, 3H) 3.36 (td, J=12.90, 3.79 Hz, 1H) 3.56-3.72 (m, 2H) 3.94-4.07 (m, 2H) 4.46 (s, 6H) 6.65-6.78 (m, 2H) 7.72 (dd, J=8.56, 0.73 Hz, 1H) 8.31 (d, J=8.56 Hz, 1H) 9.24 (br s, 1H)

Similarly prepared was:

### Intermediate 115 tert-butyl (S)-((5-(4-((N,N-diethylsulfamoyl)methyl)-6-(3-ethyl-morpholino)pyrimidin-2-yl)-1H-pyrrolo[3,2-b]pyridin-2-yl)methyl)(methyl)carbamate

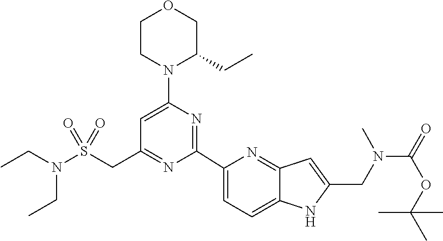 [SMILES: CC[C@H]1COCCN1c1cc(CS(=O)(=O)N(CC)CC)nc(-c2ccc3[nH]c(CN(C)C(=O)OC(C)(C)C)cc3n2)n1]

LCMS (System A, UV, ESI): Rₜ=1.20 min, [M+H]⁺ 602. ¹H NMR (400 MHz, CHLOROFORM-d) δ ppm 0.91-1.04 (m, 3H) 1.14 (t, J=7.09 Hz, 6H) 1.53 (s, 9H) 1.75-2.00 (m, 4H) 2.92 (s, 3H) 3.19-3.27 (m, 4H) 3.35 (td, J=12.90, 3.79 Hz, 1H) 3.55-3.78 (m, 2H) 3.91-4.09 (m, 2H) 4.39-4.46 (m, 2H) 4.50 (br s, 2H) 6.71 (s, 1H) 6.74 (s, 1H) 7.71 (d, 8.56 Hz, 1H) 8.31 (m, J8.56 Hz, 1H) 8.99-9.37 (m, 1H)

### EXAMPLES

### Example 1 [(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine

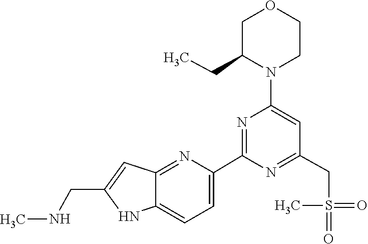 [SMILES: CC[C@H]1COCCN1c1cc(CS(C)(=O)=O)nc(-c2ccc3[nH]c(CNC)cc3n2)n1]

HCl (4M in 1,4-Dioxane) (250 μl, 1.000 mmol) was added to tert-butyl (S)-((5-(4-(3-ethylmorpholino)-6-((methylsulfonyl)methyl)pyrimidin-2-yl)-1H-pyrrolo[3,2-b]pyridin-2-yl)methyl)(methyl)carbamate (42 mg, 0.077 mmol) in 1,4-Dioxane (1000 μl). The reaction mixture was heated to 50° C. and stirred at 50° C. under nitrogen for 2 h.

The reaction mixture was neutralised with 5% aqueous K2CO3 solution (15 ml) and extracted with DCM (4×20 ml). The organic phase was dried over magnesium sulfate and the volatiles removed under vacuum. The crude product was purified by reverse phase chromatography on a XBridge Prep C18 column (80 g) using the elution gradient acetonitrile in 10 mM ammonium bicarbonate 15 to 55% to yield (S)-1-(5-(4-(3-ethylmorpholino)-6-((methylsulfonyl)methyl)pyrimidin-2-yl)-1H-pyrrolo[3,2-b]pyridin-2-yl)-Nmethylmethanamine (21 mg, 0.047 mmol, 61.3% yield).

¹H NMR (400 MHz, CHLOROFORM-d) δ 9.50 (br d, J=1.96 Hz, 1H), 8.24 (d, J=8.56 Hz, 1H), 7.68 (d, J=8.56 Hz, 1H), 6.63 (d, J=11.00 Hz, 2H), 4.36-4.48 (m, 2H), 4.12-4.33 (m, 1H), 3.92-4.05 (m, 4H), 3.53-3.72 (m, 2H), 3.33 (dt, J=3.55, 12.78 Hz, 1H), 3.08 (s, 3H), 2.47 (s, 3H), 1.87-1.99 (m, 1H), 1.81 (td, J=7.00, 14.12 Hz, 1H), 0.98 (t, J=7.46 Hz, 3H)

LCMS (System B, UV, ESI): Rₜ=0.35 min, [M+H]⁺ 445.3

Similarly prepared using the technique of Example 1 were:

### Example 2 (S)-(5-(4-(3-ethylmorpholino)-6-((methylsulfonyl)methyl)pyrimidin-2-yl)-1H-pyrrolo[3,2-b]pyridin-2-yl)methanol

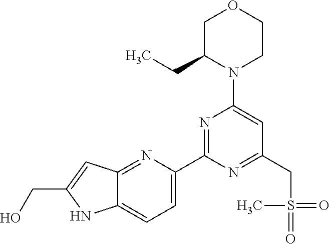 [SMILES: CC[C@H]1COCCN1c1cc(CS(C)(=O)=O)nc(-c2ccc3[nH]c(CO)cc3n2)n1]

From Intermediate 75

¹H NMR (400 MHz, DMSO-d6) δ 11.36 (d, J=1.22 Hz, 1H), 8.12 (d, J=8.56 Hz, 1H), 7.69-7.83 (m, 1H), 6.49 (d, J=0.98 Hz, 1H), 5.75 (s, 1H), 5.41 (t, J=5.62 Hz, 1H), 4.69 (d, J=5.62 Hz, 2H), 4.54 (s, 2H), 3.85-4.03 (m, 2H), 3.44-3.68 (m, 3H), 3.10-3.24 (m, 2H), 1.65-1.90 (m, 2H), 0.91 (t, J=7.46 Hz, 3H)

LCMS (System A, UV, ESI): Rₜ=0.74 min, [M+H]⁺ 432.3

### Example 3 [(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl](methyl)amine

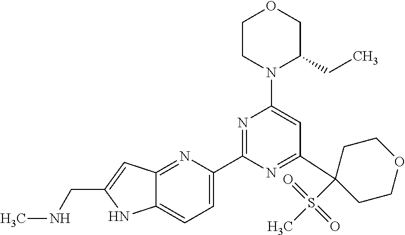 [SMILES: CC[C@H]1COCCN1c1cc(C2(S(C)(=O)=O)CCOCC2)nc(-c2ccc3[nH]c(CNC)cc3n2)n1]

From Intermediate 76

¹H NMR (CHLOROFORM-d, 400 MHz) δ 8.18 (d, 1H, J=8.6 Hz), 7.70 (d, 1H, J=8.3 Hz), 6.76 (s, 1H), 6.67 (s, 1H), 4.5-4.8 (m, 1H), 4.0-4.1 (m, 6H), 3.96 (br d, 1H, J=11.7 Hz), 3.70 (dd, 1H, J=2.9, 11.5 Hz), 3.62 (dt, 1H, J=2.8, 11.8 Hz), 3.4-3.5 (m, 2H), 3.35 (dt, 1H, J=3.8, 12.9 Hz), 2.7-2.8 (m, 5H), 2.5-2.6 (m, 3H), 2.48 (s, 3H), 1.7-2.0 (m, 2H), 0.97 (t, 3H, J=7.5 Hz)LCMS (System A, UV, ESI): Rₜ=0.45 min, [M+H]⁺ 515.3

### Example 4 (5-{4-[(3S)-3-ethylmorpholin-4-yl]-6-(4-methanesulfonyloxan-4-yl)pyrimidin-2-yl}-1H-pyrrolo[3,2-b]pyridin-2-yl)methanol

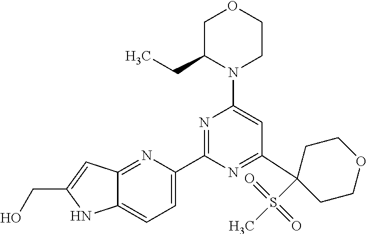 [SMILES: CC[C@H]1COCCN1c1cc(C2(S(C)(=O)=O)CCOCC2)nc(-c2ccc3[nH]c(CO)cc3n2)n1]

From Intermediate 77

¹H NMR (400 MHz, DMSO-d6) δ 11.22-11.43 (m, 1H), 8.12 (d, J=8.56 Hz, 1H), 7.76 (dd, J=0.73, 8.56 Hz, 1H), 6.95 (s, 1H), 6.49 (d, J=0.98 Hz, 1H), 5.41 (t, J=5.62 Hz, 1H), 4.69 (d, J=5.62 Hz, 2H), 3.82-4.09 (m, 4H), 3.46-3.66 (m, 2H), 3.13-3.34 (m, 8H), 2.77-2.88 (m, 2H), 2.14-2.32 (m, 2H), 1.78 (t, J=7.46 Hz, 2H), 0.88 (t, J=7.46 Hz, 3H)

LCMS (System A, UV, ESI): Rₜ=0.77 min, [M+H]⁺ 502.27

### Example 5 (5S)-5-ethyl-4-{2-[2-(hydroxymethyl)-1H-pyrrolo[3,2-b]pyridin-5-yl]-6-(methanesulfonylmethyl)pyrimidin-4-yl}morpholin-3-one

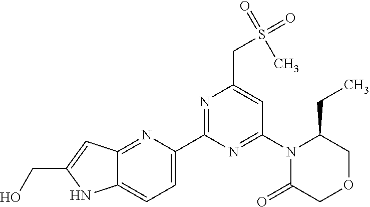 [SMILES: CC[C@H]1COCC(=O)N1c1cc(CS(C)(=O)=O)nc(-c2ccc3[nH]c(CO)cc3n2)n1]

From Intermediate 78

1H NMR (400 MHz, DMSO-d6) δ ppm 1.01 (t, J=7.46 Hz, 3H) 1.70-1.92 (m, 2H) 3.22-3.28 (m, 3H) 3.96 (dd, J=12.23, 1.71 Hz, 1H) 4.14 (d, J=11.98 Hz, 1H) 4.31 (d, J=17.36 Hz, 1H) 4.41 (d, J=17.36 Hz, 1H) 4.70 (d, J=5.38 Hz, 2H) 4.83 (s, 2H) 4.88 (br d, J=9.29 Hz, 1H) 5.44 (t, J=5.62 Hz, 1H) 6.52 (dd, J=1.83, 0.86 Hz, 1H) 7.83 (dd, J=8.56, 0.98 Hz, 1H) 8.18 (d, J=8.56 Hz, 1H) 8.32 (s, 1H) 11.47 (s, 1H)

LCMS (System B, UV, ESI): Rₜ=0.50 min, [M+H]⁺ 446.1

### Example 6 {6-[(3S)-3-ethylmorpholin-4-yl]-2-{2-[(methylamino)methyl]-1H-pyrrolo[3,2-b]pyridin-5-yl}pyrimidin-4-yl}methanesulfonamide

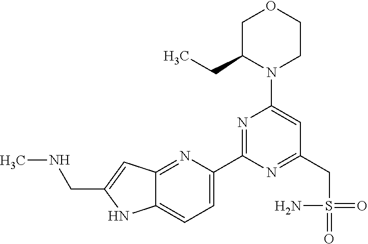 [SMILES: CC[C@H]1COCCN1c1cc(CS(N)(=O)=O)nc(-c2ccc3[nH]c(CNC)cc3n2)n1]

From Intermediate 79

¹H NMR (400 MHz, METHANOL-d4) δ 8.24 (d, J=8.56 Hz, 1H), 7.85 (dd, J=0.98, 8.56 Hz, 1H), 6.85 (s, 1H), 6.69 (d, J=0.98 Hz, 1H), 4.34-4.57 (m, 3H), 3.91-4.05 (m, 4H), 3.49-3.74 (m, 2H), 3.35-3.39 (m, 3H), 2.46 (s, 2H), 1.91 (dt, J=4.52, 7.52 Hz, 2H), 0.99 (t, J=7.46 Hz, 3H)

LCMS (System A, UV, ESI): Rₜ=0.74 min, [M+H]⁺ 446.3

### Example 7 (S)-1-(2-(3-ethyl-5-oxomorpholino)-6-(2-((methylamino)methyl)-1H-pyrrolo[3,2-b]pyridin-5-yl)pyridin-4-yl)-N,N-dimethylmethanesulfonamide

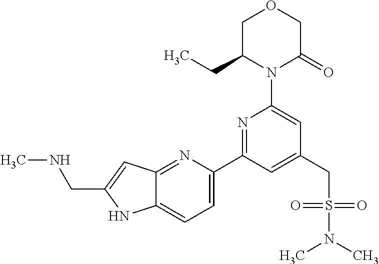 [SMILES: CC[C@H]1COCC(=O)N1c1cc(CS(=O)(=O)N(C)C)cc(-c2ccc3[nH]c(CNC)cc3n2)n1]

From Intermediate 89

¹H NMR (400 MHz, DMSO-d6) δ 11.3 (br s, 1H), 8.31-8.43 (m, 1H), 8.12 (d, J=8.56 Hz, 1H), 7.91 (d, J=1.22 Hz, 1H), 7.81 (dd, J=0.86, 8.44 Hz, 1H), 6.51 (d, J=0.73 Hz, 1H), 4.73-4.81 (m, 1H), 4.65 (d, J=4.89 Hz, 2H), 4.20-4.44 (m, 2H), 3.96-4.17 (m, 2H), 3.86 (s, 2H), 2.81 (s, 6H), 2.34 (s, 3H), 1.54-1.82 (m, 2H), 0.91 (t, J=7.46 Hz, 3H)

LCMS (System B, UV, ESI): Rₜ=0.45 min, [M+H]⁺ 487.3

### Example 8 1-(5-(6-((2R,3R)-2,3-dimethylmorpholino)-4-((methylsulfonyl)methyl)pyridin-2-yl)-1Hpyrrolo[3,2-b]pyridin-2-yl)-Nmethylmethanamine

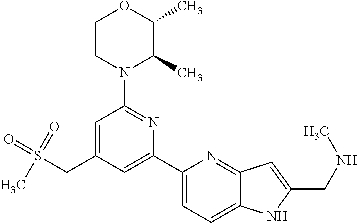 [SMILES: CNCc1cc2nc(-c3cc(CS(C)(=O)=O)cc(N4CCO[C@H](C)[C@H]4C)n3)ccc2[nH]1]

From Intermediate 91

1H NMR (400 MHz, METHANOL-d₄) δ 8.17 (d, J=8.6 Hz, 1H), 7.83 (d, J=8.6 Hz, 1H), 7.69 (d, J=0.7 Hz, 1H), 6.84 (s, 1H), 6.64 (s, 1H), 4.48 (s, 2H), 4.31 (dd, J=2.0, 6.6 Hz, 1H), 4.19-4.00 (m, 2H), 3.98-3.87 (m, 3H), 3.78 (dd, J=2.8, 11.4 Hz, 1H), 3.42-3.35 (m, 1H), 3.00 (s, 3H), 2.51-2.45 (m, 3H), 1.43 (d, J=6.6 Hz, 3H), 1.33 (d, J=6.8 Hz, 4H)

LCMS (System A, UV, ESI): Rₜ=0.86 min, [M+H]⁺ 444.3

### Example 9 1-{6-[(3S)-3-ethylmorpholin-4-yl]-2-[2-(hydroxymethyl)-1H-pyrrolo[3,2-b]pyridin-5-yl]pyrimidin-4-yl}-N,N-dimethylmethanesulfonamide

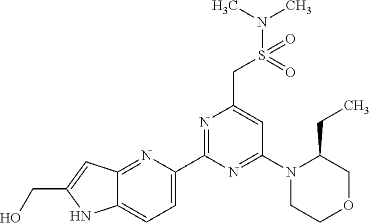 [SMILES: CC[C@H]1COCCN1c1cc(CS(=O)(=O)N(C)C)nc(-c2ccc3[nH]c(CO)cc3n2)n1]

From Intermediate 80

¹H NMR (400 MHz, CHLOROFORM-d) δ 9.90 (br s, 1H), 8.13 (d, J=8.56 Hz, 1H), 7.53 (d, J=8.56 Hz, 1H), 6.68 (s, 1H), 6.38 (s, 1H), 4.81 (s, 2H), 4.34 (s, 3H), 3.90-4.17 (m, 3H), 3.49-3.69 (m, 2H), 3.18-3.44 (m, 1H), 2.79 (s, 6H), 1.74-1.98 (m, 2H), 0.97 (t, J=7.46 Hz, 3H)

LCMS (System A, UV, ESI): Rₜ=0.81 min, [M+H+] 461.3

### Example 10 1-{6-[(3S)-3-ethylmorpholin-4-yl]-2-[2-(hydroxymethyl)-1H-pyrrolo[3,2-b]pyridin-5-yl]pyrimidin-4-yl}-N-methylmethanesulfonamide

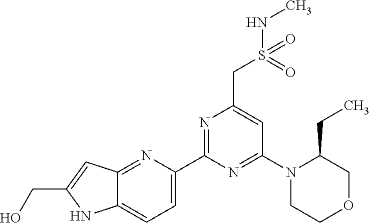 [SMILES: CC[C@H]1COCCN1c1cc(CS(=O)(=O)NC)nc(-c2ccc3[nH]c(CO)cc3n2)n1]

From Intermediate 81

¹H NMR (400 MHz, CHLOROFORM-d) δ 10.05 (br s, 1H), 7.99 (d, J=8.31 Hz, 1H), 7.49 (d, J=8.56 Hz, 1H), 6.52 (s, 1H), 6.19 (s, 1H), 4.68 (s, 2H), 4.14-4.41 (m, 3H), 3.87-4.14 (m, 3H), 3.47-3.66 (m, 3H), 3.12-3.38 (m, 1H), 2.73 (s, 3H), 1.89 (br dd, J=6.97, 14.55 Hz, 1H), 1.64-1.81 (m, 1H), 0.94 (t, J=7.46 Hz, 3H)

LCMS (System A, UV, ESI): Rₜ=0.76 min, [M+H]⁺ 447.2

### Example 11 1-{6-[(3S)-3-ethylmorpholin-4-yl]-2-{2-methyl-1H-pyrrolo[3,2-b]pyridin-5-yl}pyrimidin-4-yl}-N-methylmethanesulfonamide

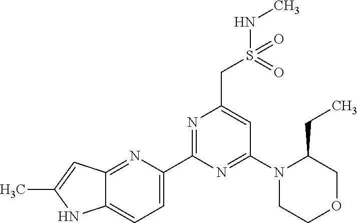 [SMILES: CC[C@H]1COCCN1c1cc(CS(=O)(=O)NC)nc(-c2ccc3[nH]c(C)cc3n2)n1]

From Intermediate 82

¹H NMR (400 MHz, CHLOROFORM-d) δ 8.17 (br d, J=8.07 Hz, 1H), 7.63 (br d, J=7.58 Hz, 1H), 6.53 (s, 1H), 6.47 (br s, 1H), 4.19-4.46 (m, 4H), 3.93-4.06 (m, 2H), 3.54-3.81 (m, 2H), 3.35 (br t, J=12.84 Hz, 1H), 2.95 (s, 3H), 2.50 (br s, 3H), 1.88-2.14 (m, 1H), 1.60-1.88 (m, 1H), 1.00 (br t, J=6.97 Hz, 3H)

LCMS (System A, UV, ESI): Rₜ=0.87 min, [M+H]⁺ 431.2

### Example 12 (3S)-3-ethyl-4-[6-(methanesulfonylmethyl)-2-{2-methyl-1H-pyrrolo[3,2-b]pyridin-5-yl}pyrimidin-4-yl]morpholine

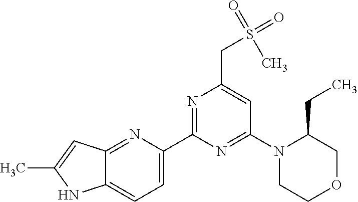 [SMILES: CC[C@H]1COCCN1c1cc(CS(C)(=O)=O)nc(-c2ccc3[nH]c(C)cc3n2)n1]

To a solution of (S)-4-(2-chloro-6-((methylsulfonyl)methyl)pyrimidin-4-yl)-3-ethylmorpholine (60 mg, 0.188 mmol), K2CO3 (51.9 mg, 0.375 mmol), palladium(II) acetate (4.21 mg, 0.019 mmol) and tricyclohexylphosphine (7.4 mg, 0.026 mmol) in 1,4-Dioxane (1.9 ml) sodium 2-methyl-1H-pyrrolo[3,2-b]pyridine-5-sulfinate (40.9 mg, 0.188 mmol) was added. The reaction vial was sealed, purged with nitrogen and left to vacuum (for 1 minute) five times. The reaction mixture was heated to 150° C. and left stirring at 150° C. overnight. palladium(II) acetate (5 mg, 0.022 mmol) was then added and the reaction mixture was sealed, heated to 150° C. and left stirring at 150° C. for 3 hours. The reaction mixture was filtered and purified using MDAP (method A, Formic). Desired fractions were combined, neutralised to ˜ pH 9, extracted with DCM:MeOH ((9:1 ratio) (3×10 ml)) and concentrated under reduced pressure. This was dissolved in MeOH:DMSO 1:1 and purified again using MDAP (HPH, method B). Desired fractions were combined and concentrated under reduced pressure to reveal (3S)-3-ethyl-4-[6-(methanesulfonylmethyl)-2-{2-methyl-1H-pyrrolo[3,2-b]pyridin-5-yl}pyrimidin-4-yl]morpholine

¹H NMR (CHLOROFORM-d, 400 MHz) δ 8.28 (br s, 1H), 8.23 (d, 1H, J=8.3 Hz), 7.68 (d, 1H, J=8.3 Hz), 6.64 (s, 1H), 6.60 (s, 1H), 4.4-4.5 (m, 2H), 4.1-4.4 (m, 2H), 4.0-4.1 (m, 1H), 3.99 (d, 1H, J=11.7 Hz), 3.7-3.8 (m, 1H), 3.6-3.7 (m, 1H), 3.3-3.4 (m, 1H), 3.10 (s, 3H), 2.54 (s, 3H), 1.9-2.0 (m, 1H), 1.8-1.9 (m, 1H), 1.00 (t, 3H, J=7.5 Hz)

LCMS (System A, UV, ESI): Rₜ=0.83 min, [M+H]⁺ 416.3

Similarly prepared using the technique of Example 12 was:

### Example 13 (3S)-3-ethyl-4-[6-(4-methanesulfonyloxan-4-yl)-2-{2-methyl-1H-pyrrolo[3,2-b]pyridin-5-yl}pyrimidin-4-yl]morpholine

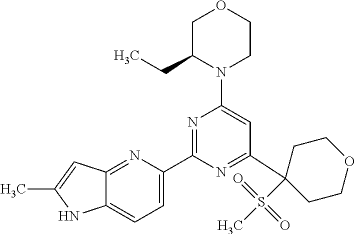 [SMILES: CC[C@H]1COCCN1c1cc(C2(S(C)(=O)=O)CCOCC2)nc(-c2ccc3[nH]c(C)cc3n2)n1]

From Intermediate 21 and Intermediate 39

¹H NMR (400 MHz, CHLOROFORM-d) δ 8.48 (br s, 1H), 8.15 (d, J=8.56 Hz, 1H), 7.64 (dd, J=0.86, 8.44 Hz, 1H), 6.76 (s, 1H), 6.54 (s, 1H), 3.92-4.17 (m, 5H), 3.56-3.79 (m, 2H), 3.30-3.55 (m, 3H), 2.68-2.84 (m, 4H), 2.49-2.62 (m, 4H), 2.03 (s, 1H), 1.71-1.97 (m, 2H), 1.68 (s, 3H), 0.97 (t, J=7.58 Hz, 3H)

LCMS (System B, UV, ESI): Rt=0.62 min, [M+H+] 486.2.

### Example 14 (5S)-5-ethyl-4-[6-(methanesulfonylmethyl)-2-{2-methyl-1H-pyrrolo[3,2-b]pyridin-5-yl}pyrimidin-4-yl]morpholin-3-one

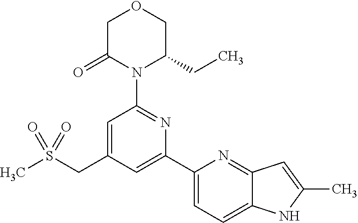

Sodium 2-methyl-1H-pyrrolo[3,2-b]pyridine-5-sulfinate (87 mg, 0.360 mmol), K2CO3 (96 mg, 0.695 mmol), palladium(II) acetate (9 mg, 0.040 mmol) and PMe(tBu)2 HBF4 (13 mg, 0.052 mmol) was added to a solution of (S)-4-(2-chloro-6-((methylsulfonyl)-methyl)pyrimidin-4-yl)-5-ethylmorpholin-3-one (100 mg, 0.300 mmol) in dry, 1,4-Dioxane (2 mL). The resulting mixture was sealed and degassed for 10 mins prior to heating at 150° C. with stirring for 3.5 hr. The reaction mixture was allowed to cool and stood at rt overnight.

Aqueous ETDA solution (0.5M, 10 mL) and DCM (10 mL) were added. The resulting biphasic mixture was filtered through a celite cartridge (2.5 g) eluting with DCM (10 mL) and EDTA (0.5M, 5 mL). The filtrate was collected, diluted with water (5 mL) and the phases separated. The aqueous phase was extracted with additional DCM (2×10 mL). The organic phase was filtered through an hydrophobic frit and the volatiles removed under reduced pressure to give the crude protected product.

The material was taken in MeOH:DMSO (1 mL) and purified by MDAP (HpH modifier, extended method B). Appropriate fractions were concentrated under nitrogen to give (S)-5-ethyl-4-(2-(2-methyl-1H-pyrrolo[3,2-b]pyridin-5-yl)-6-((methylsulfonyl)methyl)pyrimidin-4-yl)morpholin-3-one (17.1 mg, 0.039 mmol, 13.02% yield) as an off white solid. 1H NMR (400 MHz, DMSO-d6) δ ppm 1.01 (t, J=7.46 Hz, 3H) 1.69-1.90 (m, 2H) 2.47 (d, =0.73 Hz, 3H) 3.22-3.26 (m, 3H) 3.96 (br d, J=10.27 Hz, 1H) 4.14 (d, J=11.98 Hz, 1H) 4.31 (d, J=17.36 Hz, 1H) 4.41 (d, J=17.36 Hz, 1H) 4.83 (s, 2H) 4.88 (br d, J=8.31 Hz, 1H) 6.38 (br quin, J=1.20 Hz, 1H) 7.77 (dd, J=8.44, 0.86 Hz, 1H) 8.14 (d, J=8.31 Hz, 1H) 8.31 (s, 1H) 11.37 (s, 1H)

LCMS (System B, UV, ESI): Rt=0.55 min, [M+H+] 430.1

Similarly prepared using the technique of Example 14 was:

### Example 15 (5S)-5-ethyl-4-(2-{2-ethyl-1H-pyrrolo[3,2-b]pyridin-5-yl}-6-(methanesulfonylmethyl)pyrimidin-4-yl)morpholin-3-one

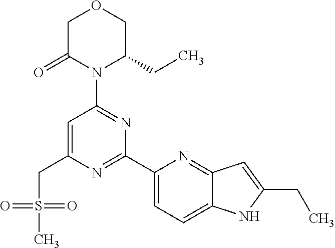 [SMILES: CCc1cc2nc(-c3nc(CS(C)(=O)=O)cc(N4C(=O)COC[C@@H]4CC)n3)ccc2[nH]1]

From Intermediate 22 and Intermediate 95

1H NMR (400 MHz, CHLOROFORM-d) δ ppm 1.08 (t, J=7.46 Hz, 3H) 1.40 (t, J=7.58 Hz, 3H) 1.82-2.05 (m, 3H) 2.89 (q, J=7.58 Hz, 2H) 3.17 (s, 3H) 3.90 (dd, J=12.23, 1.47 Hz, 1H) 4.20 (d, J=12.23 Hz, 1H) 4.32 (d, J=17.36 Hz, 1H) 4.46 (d, J=17.36 Hz, 1H) 4.57 (d, J=1.71 Hz, 2H) 4.97 (br d, J=10.03 Hz, 1H) 6.65 (s, 1H) 7.78 (d, J=8.56 Hz, 1H) 8.21 (d, J=8.31 Hz, 1H) 8.44 (s, 1H)

LCMS (System B, UV, ESI): Rt=0.59 min, [M+H+] 444.2.

### Example 16 1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide

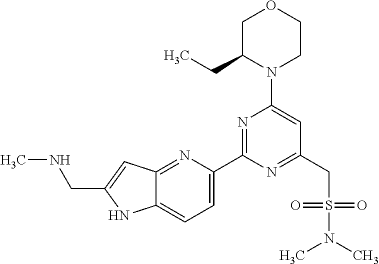 [SMILES: CC[C@H]1COCCN1c1cc(CS(=O)(=O)N(C)C)nc(-c2ccc3[nH]c(CNC)cc3n2)n1]

A solution of (S)-1-(2-chloro-6-(3-ethylmorpholino)pyrimidin-4-yl)-N,N-dimethylmethanesulfonamide (270 mg, 0.686 mmol), sodium 2-(((tert-butoxycarbonyl)(methyl)amino)methyl)-1Hpyrrolo[3,2-b]pyridine-5-sulfinate (300 mg, 0.864 mmol), K2CO3 (190 mg, 1.373 mmol), palladium(II) acetate (15.41 mg, 0.069 mmol) and tricyclohexylphosphine (28.9 mg, 0.103 mmol) in dry, thoroughly degassed 1,4-Dioxane (15 mL) was stirred at 150° C. for 20 h. EDTA (10 mL) was added and the reaction mixture diluted with EtOAC. The reaction mixture was filtered through celite (10 g) and the cartridge washed with water (10 mL) and EtOAc (10 mL). The reaction mixture was partitioned between EtOAc (50 mL) and water (50 mL). The aqueous phase was re-extracted with EtOAC (50 mL) and the combined organic phase filtered through a hydrophobic frit, and concentrated in vacuo to afford a yellow gum.

The sample was loaded in dichloromethane (1 mL) and purified on silica (Si) 20 g and eluted using a 0-100% 3:1 EtOAC:EtOH gradient over 30 mins. The appropriate fractions were combined and evaporated in vacuo to give tert-butyl (S)-((5-(4-((N,N-dimethylsulfamoyl)methyl)-6-(3-ethylmorpholino)pyrimidin-2-yl)-1H-pyrrolo[3,2-b]pyridin-2-yl)methyl)(methyl)carbamate (103 mg, 0.180 mmol, 26.2% yield). tert-butyl (S)-((5-(4-((N,N-dimethylsulfamoyl)methyl)-6-(3-ethylmorpholino)pyrimidin-2-yl)-1H-pyrrolo[3,2-b]pyridin-2-yl)methyl)(methyl)carbamate was suspended in 1,4-Dioxane (1 mL) and treated with HCl 4M in Dioxane (1.716 mL, 6.86 mmol). The reaction mixture was stirred for 1 h prior to concentration in vacuo.

The residue was suspended in 1:1 DMSO:MeOH purified by reverse phase chromatography (C18) using XSelect CSH column and eluted using Acetonitrile Water with a formic acid modifier. The solvent was evaporated in vacuo to give the required product as the formate salt. The salt was suspended in MeOH (1 mL) and applied to a pre-conditioned Aminopropyl column (20 g). The product was eluted with MeOH (3 column volumes) and concentrated in vacuo to afford (S)-1-(6-(3-ethylmorpholino)-2-(2-((methylamino)methyl)-1Hpyrrolo[3,2-b]pyridin-5-yl)pyrimidin-4-yl)-N,N-dimethylmethanesulfonamide (26.2 mg, 0.055 mmol, 8.06% yield) as a pale yellow solid.

¹H NMR (400 MHz, METHANOL-d₄) δ 8.29 (d, J=8.56 Hz, 1H), 7.86 (dd, J=0.86, 8.44 Hz, 1H), 6.88 (s, 1H), 6.71 (d, J=0.73 Hz, 1H), 4.82 (s, 2H), 4.35-4.60 (m, 2H), 3.92-4.09 (m, 4H), 3.71 (dd, J=3.18, 11.74 Hz, 1H), 3.62 (br d, J=3.18 Hz, 1H), 3.538-3.66 (dt, J=3.18, 11.74 Hz, 1H), 3.37 (m, 1H), 2.87 (s, 6H), 2.49 (s, 3H), 1.91 (t, J=7.46 Hz, 2H), 0.98 (t, J=7.46 Hz, 3H).

LCMS (System B, UV, ESI): Rₜ=0.43 min, [M+H]⁺ 473.

### Example 16A 1-{6-[(3S)-3-ethylmorpholin-4-yl}-2-{2-[(methylamino)methyl]-1H-pyrrolo[3,2-b]pyridin-5-yl}pyrimidin-4-yl]-N,N-dimethylmethanesulfonamide

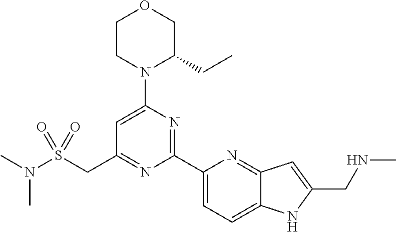

tert-butyl (S)-((5-(4-((N,N-dimethylsulfamoyl)methyl)-6-(3-ethylmorpholino)pyrimidin-2-yl)-1H-pyrrolo[3,2-b]pyridin-2-yl)methyl)(methyl)carbamate (37 mg, 0.064 mmol) was dissolved in 0.4 mL DCM and TFA (200 μl, 2.60 mmol) was added. The reaction mixture was stood at rt for 15 min. The reaction mixture was blown down under nitrogen and partitioned between DCM (8 ml) and 5% w/v K₂CO₃ solution (8 ml). The organic layer was separated and the aqueous layer was back-extracted with DCM (8 ml). The organic layers were combined, dried over a hydrophobic frit, and concentrated under a stream of nitrogen. The residue was dissolved in DMSO (0.8 ml) and eluted on an XSelect column in 10 mM ammonium bicarbonate with a gradient of 15-55% acetonitrile over 25 min. The desired fractions were concentrated under reduced pressure and a stream of nitrogen to give (S)-1-(6-(3-ethylmorpholino)-2-(2-((methylamino)methyl)-1H-pyrrolo[3,2-b]pyridin-5-yl)pyrimidin-4-yl)-N,N-dimethylmethanesulfonamide (6.1 mg, 0.013 mmol, 19.97% yield) as a slightly yellow glass. ¹H NMR (400 MHz, CHLOROFORM-d) 6=8.28 (d, J=8.6 Hz, 1H), 7.69 (dd, J=1.0, 8.6 Hz, 1H), 6.70-6.62 (m, 2H), 4.43 (s, 3H), 4.29-4.13 (m, 1H), 4.06-3.94 (m, 4H), 3.72-3.56 (m, 2H), 3.39-3.26 (m, 1H), 2.84 (s, 6H), 2.63 (s, 2H), 2.47 (s, 3H), 1.99-1.88 (m, 1H), 1.87-1.77 (m, 1H), 0.98 (t, J=7.5 Hz, 3H). LCMS (System A, UV, ESI): Rₜ=0.85 min, [M+H]⁺ 474.

### Example 17 (3S)-3-ethyl-4-[6-(4-methanesulfonylpiperidin-4-yl)-2-{2-methyl-1H-pyrrolo[3,2-b]pyridin-5-yl}pyrimidin-4-yl]morpholine

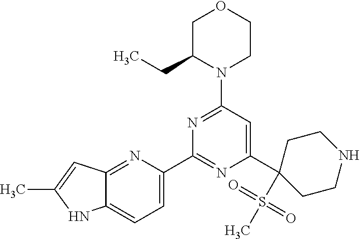 [SMILES: CC[C@H]1COCCN1c1cc(C2(S(C)(=O)=O)CCNCC2)nc(-c2ccc3[nH]c(C)cc3n2)n1]

A mixture of tert-butyl (S)-4-(2-chloro-6-(3-ethylmorpholino)pyrimidin-4-yl)-4-(methylsulfonyl)piperidine-1-carboxylate (46 mg, 0.094 mmol), sodium 2-methyl-1H-pyrrolo[3,2-b]pyridine-5-sulfinate (25.9 mg, 0.113 mmol), K2CO3 (26.0 mg, 0.188 mmol), palladium(II) acetate (2.112 mg, 9.41 μmol) and PMe(tBu)2 HBF4 (3.50 mg, 0.014 mmol) in dry, degassed 1,4-Dioxane (5 mL). The resulting mixture was sealed and degassed for a further 5 mins prior to heating at 150° C. with stirring for 24 h.

The mixture was cooled and aqueous ETDA solution (5%, 10 mL) was added. The resulting mixture was filtered through a celite cartridge (2.5 g) eluting with DCM (2×10 mL). The filtrate was collected and partitioned between DCM (20 ML) and water (10 mL) and the phases were separated. The aqueous phase was extracted with additional DCM (10 mL). The organic phase was filtered through an hydrophobic frit and the volatiles removed under reduced pressure to give the crude protected product. The residue was suspended in 1,4-Dioxane (5 mL) and treated with HCl 4M in Dioxane (2 mL, 8.00 mmol). The reaction mixture was stirred for 2 h.

The residue was suspended in DMSO purified by reverse phase chromatography (C18) using Zorbax SB-Phenyl, 30×150 mm, 5 um column and eluted using Acetonitrile Water with a TFA modifier. The solvent was evaporated in vacuo to give the required product as the TFA salt. The salt was suspended in MeOH (1 mL) and applied to a pre-conditioned Aminopropyl column (20 g). The product was eluted with MeOH (3 column volumes) and concentrated in vacuo to afford (S)-3-ethyl-4-(2-(2-methyl-1H-pyrrolo[3,2-b]pyridin-5-yl)-6-(4-(methylsulfonyl)piperidin-4-yl)pyrimidin-4-yl)morpholine (2.7 mg, 5.57 μmol, 5.92% yield).

¹H NMR (400 MHz, METHANOL-d₄) δ 8.56-8.78 (m, J=8.31 Hz, 1H), 8.31-8.55 (m, J=8.31 Hz, 1H), 7.17 (s, 1H), 6.81 (s, 1H), 4.84-5.10 (m, 1H), 3.90-4.15 (m, 2H), 3.50-3.76 (m, 4H), 3.35-3.50 (m, 2H), 3.20-3.30 (m, 2H), 2.92-3.09 (m, 2H), 2.87 (s, 3H), 2.72 (s, 3H), 2.43-2.69 (m, 3H), 1.81-2.09 (m, 2H), 0.98 (t, J=7.46 Hz, 3H)

LCMS (System B, UV, ESI): Rt=0.42 min, [M+H+] 485.2.

### Example 18 (S)-3-ethyl-4-(6-(2-methyl-1H-pyrrolo[3,2-b]pyridin-5-yl)-4-(4-(methylsulfonyl)piperidin-4-yl)pyridin-2-yl)morpholine

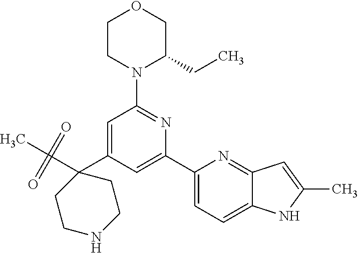

Tert-butyl (S)-4-(2-(3-ethylmorpholino)-6-(2-methyl-1H-pyrrolo[3,2-b]pyridin-5-yl)pyridin-4-yl)-4-(methylsulfonyl)piperidine-1-carboxylate (46 mg, 0.079 mmol) was dissolved in Dichloromethane (DCM) (1.0 mL). TFA (0.500 mL, 6.49 mmol) was added and the resulting mixture was stirred at RT for 4 h

The resulting solution was quenched by slow addition of sat. aq. sodium bicarbonate (20 mL). The product was then extracted with DCM (2×10 mL). The combined organics were passed through a hydrophobic frit and concentrated under reduced pressure to give crude product.

The crude product was purified by reverse phase chromatography using 10 mM ammonium bicarbonate in water adjusted to pH 10 with ammonia solution (A) and CH₃CN (B) using the gradient of 15-55% (B) and concentrated under reduced pressure to give (S)-3-ethyl-4-(6-(2-methyl-1H-pyrrolo[3,2-b]pyridin-5-yl)-4-(4-(methylsulfonyl)piperidin-4-yl)pyridin-2-yl)morpholine (16 mg).

¹H NMR (400 MHz, DMSO-d₆) δ 11.14-11.27 (m, 1H), 8.12 (d, J=8.56 Hz, 1H), 7.88 (d, J=0.98 Hz, 1H), 7.71 (dd, J=0.86, 8.44 Hz, 1H), 6.82 (s, 1H), 6.28-6.44 (m, 1H), 4.10-4.27 (m, 2H), 3.88-4.02 (m, 2H), 3.48-3.67 (m, 2H), 3.15 (dt, J=3.67, 12.72 Hz, 1H), 2.91-3.04 (m, 2H), 2.69 (s, 3H), 2.57-2.67 (m, 2H), 2.46 (d, J=0.73 Hz, 5H), 2.02-2.18 (m, 2H), 1.74-1.87 (m, 1H), 1.56 (s, 1H), 0.90 (t, J=7.46 Hz, 3H)

LCMS (System B, UV, ESI): Rₜ=0.61 min, [M+H]⁺ 432

### Example 19 (S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine

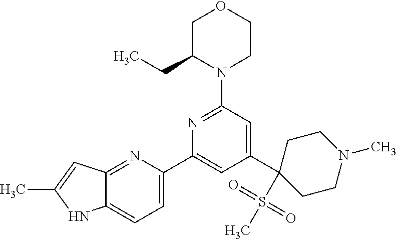

A mixture of (S)-4-(6-bromo-4-(1-methyl-4-(methylsulfonyl)piperidin-4-yl)pyridin-2-yl)-3-ethylmorpholine (110 mg, 0.246 mmol), sodium 2-methyl-1H-pyrrolo[3,2-b]pyridine-5-sulfinate (67.9 mg, 0.296 mmol), K2CO3 (68.1 mg, 0.493 mmol), palladium(II) acetate (5.53 mg, 0.025 mmol) and PMe(tBu)2 HBF4 (9.17 mg, 0.037 mmol) was suspended in dry, degassed 1,4-Dioxane (5 mL). The resulting mixture was sealed and degassed for a further 5 mins prior to heating at 150° C. with stirring for 24 h.

The mixture was cooled and aqueous ETDA solution (5%, 10 mL) was added. The resulting mixture was filtered through a celite cartridge (2.5 g) eluting with DCM (2×10 mL). The filtrate was collected and partitioned between DCM (20 ML) and water (10 mL) and the phases were separated. The aqueous phase was extracted with additional DCM (10 mL). The organic phase was filtered through an hydrophobic frit and the volatiles removed under reduced pressure to give the crude product which was purified by reverse phase (C18) using XSelect CSH column and eluted using Acetonitrile Water with an ammonium carbonate modifier. The solvent was evaporated in vacuo to give the required product (S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine (34.5 mg, 28.1% yield).

¹H NMR (400 MHz, CHLOROFORM-d) δ 8.24 (d, J=8.56 Hz, 1H), 8.09-8.20 (m, 1H), 8.04 (d, J=1.22 Hz, 1H), 7.65 (dd, J=0.86, 8.44 Hz, 1H), 6.81 (d, J=1.22 Hz, 1H), 6.50 (s, 1H), 4.11-4.23 (m, 2H), 3.90-4.10 (m, 2H), 3.60-3.79 (m, 2H), 3.32 (dt, J=3.79, 12.65 Hz, 1H), 2.80-3.04 (m, 2H), 2.58-2.69 (m, 5H), 2.54 (s, 3H), 2.07-2.31 (m, 5H), 1.90-2.04 (m, 2H), 1.59-1.85 (m, 2H), 0.99 (t, J=7.46 Hz, 3H)

LCMS (SystemB, UV, ESI): Rₜ=0.44 min, [M+H]⁺ 498

Similarly prepared using the technique of Example 19 were:

### Example 20 (S)-(5-(6-(3-ethylmorpholino)-4-(1-methyl-4-(methylsulfonyl)piperidin-4-yl)pyridin-2-yl)-1H-pyrrolo[3,2-b]pyridin-2-yl)methanol

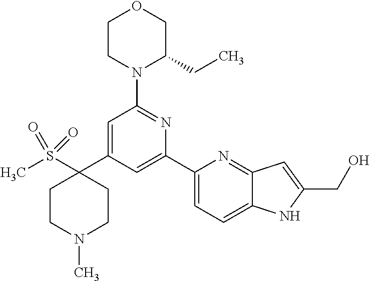 [SMILES: CC[C@H]1COCCN1c1cc(C2(S(C)(=O)=O)CCN(C)CC2)cc(-c2ccc3[nH]c(CO)cc3n2)n1]

From Intermediate 71 and Intermediate 20

¹H NMR (400 MHz, CHLOROFORM-d) δ 8.78 (br s, 1H), 8.28 (d, J=8.56 Hz, 1H), 8.03 (d, J=0.73 Hz, 1H), 7.72 (d, J=8.56 Hz, 1H), 6.80 (d, J=0.98 Hz, 1H), 6.60 (s, 1H), 3.95-4.29 (m, 4H), 3.65-3.86 (m, 2H), 3.33 (dt, J=3.55, 12.65 Hz, 1H), 2.87-3.04 (m, 2H), 2.55-2.78 (m, 6H), 2.08-2.35 (m, 5H), 1.85-2.06 (m, 2H), 1.50-1.84 (m, 4H), 0.99 (t, J=7.46 Hz, 3H).

LCMS (SystemB, UV, ESI): Rₜ=0.40 min, [M+H]⁺ 514.

### Example 21 (S)-3-ethyl-4-(6-(2-methyl-1H-pyrrolo[3,2-b]pyridin-5-yl)-4-(4-(methylsulfonyl)tetra-hydro-2H-pyran-4-yl)pyridin-2-yl)morpholine

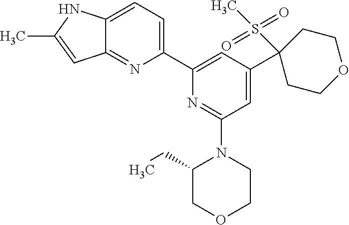 [SMILES: CC[C@H]1COCCN1c1cc(C2(S(C)(=O)=O)CCOCC2)cc(-c2ccc3[nH]c(C)cc3n2)n1]

A solution of (S)-4-(6-chloro-4-(4-(methylsulfonyl)tetrahydro-2H-pyran-4-yl)pyridin-2-yl)-3-ethylmorpholine (84 mg, 0.216 mmol), 2-methyl-1H-pyrrolo[3,2-b]pyridine-5-sulfinate, Sodium salt (77.5 mg, 0.355 mmol), K2CO3 (58 mg, 0.420 mmol), tricyclohexylphosphane (19.1 mg, 0.068 mmol) and palladium(II) acetate (6.4 mg, 0.029 mmol) in 1,4-Dioxane (2000 μl) was degassed under a stream of nitrogen for 5 minutes. The reaction mixture was then sealed and heated to 150° C. for 24 hours.

The reaction mixture was diluted with EtOAc (20 mL), filtered on a pre-packed celite cartridge then washed with EtOAc (2×10 mL). The mixture was then partitioned with brine (40 mL). The organic layer was taken, dried over a hydrophobic frit, and concentrated in vacuo.

The residue was dissolved in 2 mL DMSO:MeOH (1:1) and purified by reverse phase (C18) using XSelect CSH column and eluted using Acetonitrile Water with a formic acid modifier. The fractions containing product were combined and neutralised with sodium bicarbonate and then extracted with DCM (3×40 mL). The organic layers were combined and the solvent removed under vacuum. Sample was then dissolved in MeCN (10 mL) and washed with cyclohexane (3×10 mL). The MeCN was removed under vacuum to yield (S)-3-ethyl-4-(6-(2-methyl-1H-pyrrolo[3,2-b]pyridin-5-yl)-4-(4-(methylsulfonyl)tetrahydro-2H-pyran-4-yl)pyridin-2-yl)morpholine (32 mg, 0.066 mmol, 30.6% yield) as a pale yellow solid.

¹H NMR (DMSO-d₆, 400 MHz) δ 11.22 (s, 1H), 8.12 (d, 1H, J=8.3 Hz), 7.88 (d, 1H, J=1.0 Hz), 7.72 (dd, 1H, J=0.9, 8.4 Hz), 6.84 (s, 1H), 6.3-6.5 (m, 1H), 4.2-4.3 (m, 1H), 4.1-4.2 (m, 1H), 3.9-4.0 (m, 4H), 3.6-3.7 (m, 1H), 3.5-3.6 (m, 1H), 3.32-3.37 (m, 1H), 3.1-3.3 (m, 2H), 2.74 (s, 3H), 2.6-2.7 (m, 2H), 2.46 (d, 3H, J=0.7 Hz), 2.2-2.4 (m, 2H), 1.7-1.9 (m, 1H), 1.5-1.6 (m, 1H), 0.90 (t, 3H, J=7.5 Hz)LCMS (System A, UV, ESI): Rₜ=0.99 min, [M+H]⁺ 485

### Example 22 (S)-3-ethyl-4-(6-(2-methyl-1H-pyrrolo[3,2-b]pyridin-5-yl)-4-((methylsulfonyl)methyl) pyridin-2-yl)morpholine

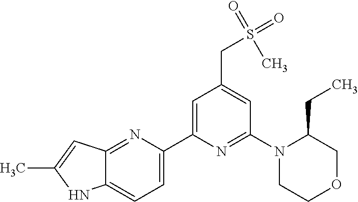 [SMILES: CC[C@H]1COCCN1c1cc(CS(C)(=O)=O)cc(-c2ccc3[nH]c(C)cc3n2)n1]

To a stirred solution of (S)-3-ethyl-4-(6-(2-methyl-1-(phenylsulfonyl)-1H-pyrrolo[3,2-b]pyridin-5-yl)-4-((methylsulfonyl)methyl)pyridin-2-yl)morpholine (38 mg, 0.069 mmol) and methanamine (2M in THF) (0.10 mL, 0.200 mmol) in anhydrous Ethanol (1.8 mL) and anhydrous Tetrahydrofuran (THF) (2.70 mL), was added Sodium hydroxide (1M in water) (0.685 mL, 0.685 mmol). The reaction mixture was stirred at 50° C. for 19 h.

The reaction mixture was diluted with saturated aqueous ammonium chloride solution (15 mL), and the aqueous phase extracted with DCM (3×25 mL). The combined organic phase was passed through a hydrophobic frit and evaporated in vacuo to afford a yellow oil.

The oil was dissolved in 1:1 DMSO:MeOH (1.0 mL) and purified by Mass Directed AutoPreparative HPLC (MDAP) on OA MDAP (Xselect CSH column 150 mm×30 mm i.d. 5 μM packing diameter at ambient temperature) eluting with solvents A/B (A: 10 mM ammonium bicarbonate in water adjusted to pH 10 with ammonia solution, B: acetonitrile), using a method B run. The product-containing fractions were combined and evaporated in vacuo to afford (S)-3-ethyl-4-(6-(2-methyl-1Hpyrrolo[3,2-b]pyridin-5-yl)-4-((methylsulfonyl)methyl)pyridin-2-yl)morpholine (20 mg, 0.048 mmol, 70.4% yield) as a pale yellow solid.

¹H NMR (400 MHz, DMSO-d6) δ 11.22 (s, 1H), 8.09 (d, J=8.31 Hz, 1H), 7.71 (d, J=8.31 Hz, 1H), 6.75 (s, 1H), 6.33 (s, 1H), 5.75 (s, 1H), 4.53 (s, 2H), 4.04-4.21 (m, 2H), 3.89-4.02 (m, 2H), 3.47-3.66 (m, 2H), 3.10-3.24 (m, 1H), 2.98 (s, 3H), 2.46 (s, 3H), 1.83 (td, J=7.73, 14.12 Hz, 1H), 1.50-1.66 (m, 1H), 0.92 (t, J=7.46 Hz, 3H)

LCMS (System A, UV, ESI): Rₜ=0.97 min, [M+H]⁺ 415

### Example 23 (S)-(2-(3-ethylmorpholino)-6-(2-methyl-1H-pyrrolo[3,2-b]pyridin-5-yl)pyridin-4-yl)methanesulfonamide

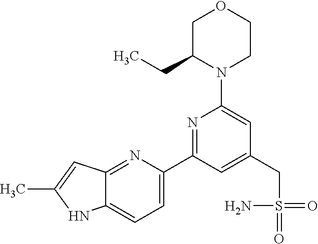 [SMILES: CC[C@H]1COCCN1c1cc(CS(N)(=O)=O)cc(-c2ccc3[nH]c(C)cc3n2)n1]

(S)-1-(2-(3-Ethylmorpholino)-6-(2-methyl-1H-pyrrolo[3,2-b]pyridin-5-yl)pyridin-4-yl)-N-(4-methoxybenzyl)methanesulfonamide (107 mg, 0.200 mmol) was dissolved in TFA (1000 μl, 12.98 mmol). The reaction mixture was sealed in a vial, and stirred at 100° C. for 10 min in the biotage microwave system.

The reaction mixture was blown down under nitrogen and partitioned between 2.5 mL EtOAc and 2.5 mL 5% w/v K2CO3 solution. The organic layer was separated, and dried over a hydrophobic frit. The aqueous layer was then stood at rt for 16 h. The organic layer was concentrated under a stream of nitrogen.

The aqueous layer was back-extracted with 2 mL EtOAc. The organic extraction was dried over a hydrophobic frit, combined with the residue from the first organic layer, diluted with 0.8 mL DMSO, and concentrated under a stream of nitrogen. The residue was diluted with a further 0.2 mL DMSO and eluted on an XSelect column in 10 mM ammonium bicarbonate with a gradient of 15-55% acetonitrile over 20 min. The desired fractions were concentrated under reduced pressure to give (S)-(2-(3-ethylmorpholino)-6-(2-methyl-1Hpyrrolo[3,2-b]pyridin-5-yl)pyridin-4-yl)methanesulfonamide (46 mg, 0.111 mmol, 55.4% yield) as a yellow solid.

¹H NMR (400 MHz, METHANOL-d₄) δ 8.06 (d, J=8.56 Hz, 1H), 7.74 (dd, J=0.73, 8.31 Hz, 1H), 7.62 (s, 1H), 6.77 (s, 1H), 6.39 (s, 1H), 4.39 (s, 2H), 4.23-4.35 (m, 1H), 4.13 (dd, J=2.20, 13.20 Hz, 1H), 3.93-4.07 (m, 2H), 3.54-3.75 (m, 2H), 3.14-3.31 (m, 1H), 2.52 (d, J=0.73 Hz, 3H), 1.82-2.09 (m, 1H), 1.58-1.82 (m, 1H), 1.00 (t, J=7.46 Hz, 3H)

LCMS (System A, UV, ESI): Rₜ=0.92 min, [M+H]⁺ 416.1

### Example 24 (S)-5-ethyl-4-(6-(2-methyl-1H-pyrrolo[3,2-b]pyridin-5-yl)-4-((methylsulfonyl) methyl)pyridin-2-yl)morpholin-3-one

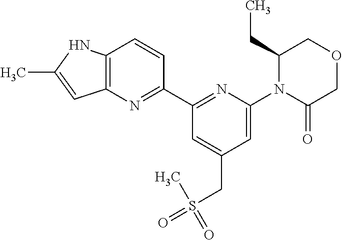 [SMILES: CC[C@H]1COCC(=O)N1c1cc(CS(C)(=O)=O)cc(-c2ccc3[nH]c(C)cc3n2)n1]

A solution of (S)-4-(6-bromo-4-((methylsulfonyl)methyl)pyridin-2-yl)-5-ethylmorpholin-3-one (99 mg, 0.262 mmol), 2-methyl-1H-pyrrolo[3,2-b]pyridine-5-sulfinate, Sodium salt (80 mg, 0.367 mmol), K2CO3 (72 mg, 0.521 mmol), tricyclohexylphosphane (20 mg, 0.071 mmol) and palladium(II) acetate (10 mg, 0.045 mmol) in 1,4-Dioxane (2.000 mL) was placed in a microwave vial and sealed. The mixture was degassed using N2/vacuum 3 times. The mixture was heated to 150° C. for 16 h.

The reaction mixture was diluted with EtOAc (10 mL), filtered on a pre-packed celite cartridge then washed with EtOAc. The mixture was then partitioned with brine (40 mL). The organic layer was taken, passed through a hydrophobic frit, and concentrated under reduced pressure to give crude product.

Crude product was purified by reverse phase chromatography using the High pH MDAP. The solvent system used was 10 mM ammonium bicarbonate in water adjusted to pH 10 with ammonia solution (A) and CH₃CN (B). The compound was dissolved in 1:1 DMSO/MeOH and run using the gradient of (30-85% (B)) over 15 mins. The fractions were concentrated under reduced pressure to give (S)-5-ethyl-4-(6-(2-methyl-1H-pyrrolo[3,2-b]pyridin-5-yl)-4-((methylsulfonyl)methyl)pyridin-2-yl)morpholin-3-one (38 mg, 0.089 mmol, 33.8% yield).

¹H NMR (400 MHz, DMSO-d₆) δ 11.33 (s, 1H), 8.35 (d, J=1.22 Hz, 1H), 8.10 (d, J=8.56 Hz, 1H), 7.93 (d, J=1.22 Hz, 1H), 7.69-7.82 (m, 1H), 6.31-6.49 (m, 1H), 4.59-4.84 (m, 2H), 4.20-4.42 (m, 2H), 3.92-4.15 (m, 2H), 3.02 (s, 3H), 2.48 (d, J=0.73 Hz, 3H), 1.75 (br dd, J=7.46, 8.93 Hz, 2H), 0.92 (t, J=7.46 Hz, 3H).

LCMS (System B, UV, ESI): Rₜ=0.51 min, [M+H]⁺ 429.

Similarly prepared using the technique of Example 24 were:

### Example 25 (S)-5-ethyl-4-(6-(2-methyl-1H-pyrrolo[3,2-b]pyridin-5-yl)-4-(4-(methylsulfonyl) tetrahydro-2H-pyran-4-yl)pyridin-2-yl)morpholin-3-one

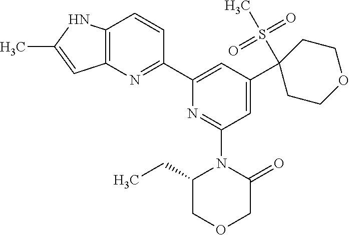 [SMILES: CC[C@H]1COCC(=O)N1c1cc(C2(S(C)(=O)=O)CCOCC2)cc(-c2ccc3[nH]c(C)cc3n2)n1]

From Intermediate 48 and Intermediate 21

¹H NMR (400 MHz, METHANOL-d₄) δ 8.46-8.53 (m, 1H), 8.18 (d, J=8.31 Hz, 1H), 8.03 (d, J=1.47 Hz, 1H), 7.80 (dd, J=0.86, 8.44 Hz, 1H), 6.44 (t, J=0.86 Hz, 1H), 4.73-4.81 (m, 1H), 4.26-4.46 (m, 2H), 4.19 (dd, J=1.47, 12.23 Hz, 1H), 3.99-4.10 (m, 3H), 3.38-3.57 (m, 2H), 2.68-2.82 (m, 5H), 2.54 (d, J=0.98 Hz, 5H), 1.73-1.96 (m, 2H), 0.97 (t, J=7.58 Hz, 3H)

LCMS (System A, UV, ESI): Rₜ=0.89 min, [M+H]⁺ 499

### Example 26 (S)-5-ethyl-4-(6-(2-(hydroxymethyl)-1H-pyrrolo[3,2-b]pyridin-5-yl)-4-(4-(methylsulfonyl)tetrahydro-2H-pyran-4-ylpyridin-2-yl)morpholin-3-one

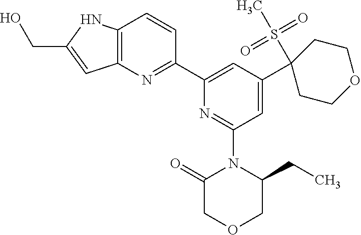 [SMILES: CC[C@H]1COCC(=O)N1c1cc(C2(S(C)(=O)=O)CCOCC2)cc(-c2ccc3[nH]c(CO)cc3n2)n1]

TBAF (1M in THF) (100 μl, 0.100 mmol) was added to a solution of (S)-5-ethyl-4-(4-(4-(methylsulfonyl)tetrahydro-2H-pyran-4-yl)-6-(2-(((triisopropylsilyl)oxy)methyl)-1Hpyrrolo[3,2-b]pyridin-5-yl)pyridin-2-yl)morpholin-3-one (60 mg, 0.089 mmol) and dry Tetrahydrofuran (THF) (1000 μl) in a sealed rbf under nitrogen and stirred 30 min.

Reaction mixture was partitioned with saturated aqueous sodium bicarbonate (10 mL) solution and EtOAc (10 mL). The aqueous layer was back extracted with EtOAc (2×10 mL).

The samples were dissolved in 1:1 DMSO:MeOH (1 mL) and purified by Mass Directed AutoPrep (HpH, method B). The solvent was removed under vacuum to yield (S)-5-ethyl-4-(6-(2-(hydroxymethyl)-1H-pyrrolo[3,2-b]pyridin-5-yl)-4-(4-(methylsulfonyl)tetrahydro-2H-pyran-4-yl)pyridin-2-yl)morpholin-3-one (30 mg, 0.058 mmol, 65.2% yield) as a white solid.

¹H NMR (400 MHz, METHANOL-d₄) δ 8.51 (d, J=1.47 Hz, 1H), 8.24 (d, J=8.56 Hz, 1H), 8.04 (d, J=1.47 Hz, 1H), 7.88 (dd, J=0.98, 8.56 Hz, 1H), 6.64 (d, J=0.98 Hz, 1H), 4.84 (s, 2H), 4.73-4.80 (m, 1H), 4.23-4.49 (m, 2H), 4.14-4.22 (m, 1H), 4.04 (br dd, J=2.81, 12.10 Hz, 3H), 3.39-3.56 (m, 2H), 2.69-2.82 (m, 5H), 2.55 (ddd, J=4.52, 12.17, 13.88 Hz, 2H), 1.70-1.96 (m, 2H), 0.96 (t, J=7.46 Hz, 3H)

LCMS (System A, UV, ESI): Rₜ=0.76 min, [M+H]⁺ 515

Similarly prepared using the technique of Example 26 were:

### Example 27 (S)-5-ethyl-4-(6-(2-(hydroxymethyl)-1H-pyrrolo[3,2-b]pyridin-5-yl)-4-((methylsulfonyl) ethyl)pyridin-2-yl)morpholin-3-one

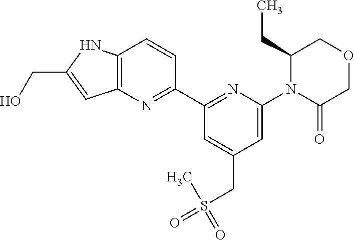 [SMILES: CC[C@H]1COCC(=O)N1c1cc(CS(C)(=O)=O)cc(-c2ccc3[nH]c(CO)cc3n2)n1]

From Intermediate 87

¹H NMR (400 MHz, DMSO-d₆) δ 11.43 (s, 1H), 8.36 (d, J=1.22 Hz, 1H), 8.14 (d, J=8.56 Hz, 1H), 7.94 (d, J=1.47 Hz, 1H), 7.83 (dd, J=0.98, 8.56 Hz, 1H), 6.51 (s, 1H), 5.45 (t, J=5.62 Hz, 1H), 4.67-4.81 (m, 5H), 4.20-4.41 (m, 2H), 3.96-4.17 (m, 2H), 3.02 (s, 3H), 1.75 (br dd, J=7.34, 9.05 Hz, 2H), 0.92 (t, J=7.46 Hz, 3H)

LCMS (System A, UV, ESI): Rₜ=0.74 min, [M+H]⁺ 445

### Example 28 (S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one

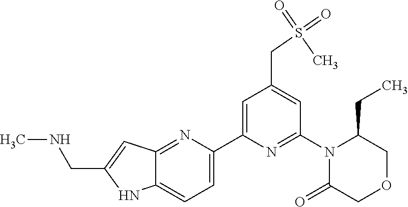 [SMILES: CC[C@H]1COCC(=O)N1c1cc(CS(C)(=O)=O)cc(-c2ccc3[nH]c(CNC)cc3n2)n1]

(S)-4-(6-Chloro-4-((methylsulfonyl)methyl)pyridin-2-yl)-5-ethylmorpholin-3-one (35 mg, 0.089 mmol), palladium(II) acetate (2.007 mg, 8.94 μmol), tricyclohexylphosphine (5.01 mg, 0.018 mmol), sodium 2-(((tert-butoxycarbonyl)(methyl)amino)methyl)-1H-pyrrolo[3,2-b]pyridine-5-sulfinate (34.2 mg, 0.098 mmol) and K2CO3 (18.53 mg, 0.134 mmol) were placed in dry 1,4-Dioxane (1 mL) and the resulting mixture was degassed under a flow of nitrogen for 5 min, sealed, and heated to 150° C. for 18 h. The mixture was cooled down and the mixture was partitioned between DCM (5 mL) and water (2 mL). The phase was separated and the aqueous was extracted with additional DCM (5 mL). The volatiles were removed under a flow of nitrogen to give a residue (61 mg) that was dissolved in Dichloromethane (DCM) (1000 μl):TFA (500 μl, 6.49 mmol) and stirred at RT for 2 h.

The volatiles were removed under a flow of nitrogen and the residue was dissolved in DCM (4 mL). This solution was washed with aqueous K2CO3 (5%, 10 mL) and the aqueous phase was extracted with additional DCM (2×5 mL). The organic phase were combined and the volatiles were removed under reduced pressure to give a residue that was purified by reverse phase chromatography on a Xbridge RP C18 column using the elution gradient acetonitrile in water 20-60% (ammonium bicarbonate modifier) to give (S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one (20 mg, 0.044 mmol, 48.9% yield) as a colourless solid.

¹H NMR (400 MHz, CHLOROFORM-d) δ 8.78-8.93 (m, 1H), 8.43 (d, J=0.98 Hz, 1H), 8.16 (d, J=8.56 Hz, 1H), 8.07 (d, J=0.73 Hz, 1H), 7.71 (d, J=8.56 Hz, 1H), 6.63 (s, 1H), 4.89 (br dd, J=2.69, 6.36 Hz, 1H), 4.28-4.51 (m, 4H), 4.19 (br d, J=11.49 Hz, 1H), 3.94-4.07 (m, 3H), 2.93 (s, 3H), 2.53 (s, 3H), 1.74-1.99 (m, 2H), 1.00 (t, J=7.46 Hz, 3H)

LCMS (System B, UV, ESI): Rₜ=0.38 min, [M+H]⁺ 458.3

### Example 29 (S)-1-(2-(3-ethyl-5-oxomorpholino)-6-(2-(hydroxymethyl)-1H-pyrrolo[3,2-b]pyridin-5-yl)pyridin-4-yl)-N,Ndimethylmethanesulfonamide

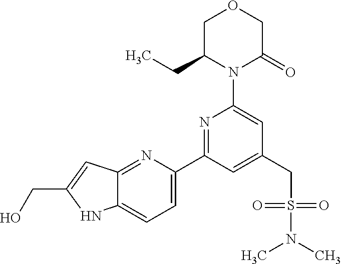 [SMILES: CC[C@H]1COCC(=O)N1c1cc(CS(=O)(=O)N(C)C)cc(-c2ccc3[nH]c(CO)cc3n2)n1]

2M HCl (aqueous solution) (1.5 ml, 3.00 mmol) was added to a solution of (S)-1-(2-(3-ethyl-5-oxomorpholino)-6-(2-(((triisopropylsilyl)oxy)methyl)-1H-pyrrolo[3,2-b]pyridin-5-yl)pyridin-4-yl)-N,N-dimethylmethanesulfonamide (181 mg, 0.287 mmol) in Tetrahydrofuran (THF) (2 ml). The reaction mixture was stirred at RT for 6 hr.

The reaction mixture was partitioned between 15 ml saturated aqueous sodium carbonate solution and 15 mL Ethyl acetate. The organic layer was separated, and the aqueous layer was back extracted with 15 ml Ethyl acetate. The organic layers were combined, dried over a hydrophobic frit, and concentrated under reduced pressure. The residue was dissolved in 1.5 mL DMSO and eluted on an XBridge column with in 10 mM ammonium bicarbonate with a gradient of 15-55% acetonitrile.

The desired fractions were concentrated under reduced pressure to give (S)-1-(2-(3-ethyl-5-oxomorpholino)-6-(2-(hydroxymethyl)-1H-pyrrolo[3,2-b]pyridin-5-yl)pyridin-4-yl)-N,N-dimethylmethanesulfonamide (14.4 mg, 0.030 mmol, 10.58% yield).

The residue was further purified to give (S)-1-(2-(3-ethyl-5-oxomorpholino)-6-(2-(hydroxymethyl)-1H-pyrrolo[3,2-b]pyridin-5-yl)pyridin-4-yl)-N,N-dimethylmethanesulfonamide.

¹H NMR (400 MHz, CHLOROFORM-d) δ 8.69-8.87 (m, 1H), 8.41 (d, J=1.22 Hz, 1H), 8.10 (d, J=8.56 Hz, 1H), 7.98 (d, J=1.22 Hz, 1H), 7.68 (dd, J=0.73, 8.56 Hz, 1H), 6.61 (s, 1H), 4.92 (s, 2H), 4.80-4.88 (m, 1H), 4.27-4.50 (m, 4H), 3.95-4.21 (m, 2H), 2.89 (s, 6H), 2.54-2.66 (m, 1H), 1.69-1.98 (m, 2H), 0.97 (t, J=7.46 Hz, 3H).

LCMS (System A, UV, ESI): Rₜ=0.82 min, [M+H]⁺ 474.2.

Similarly prepared to Example 16A were:

### Example 30 (S)—N,N-diethyl-1-(6-(3-ethylmorpholino)-2-(2-((methylamino)methyl)-1H-pyrrolo[3,2-b]pyridin-5-yl)pyrimidin-4-yl)methanesulfonamide

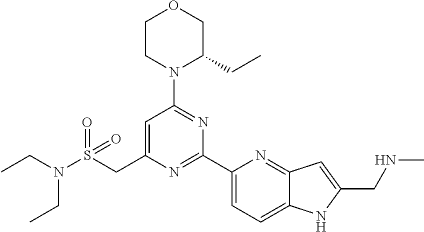 [SMILES: CC[C@H]1COCCN1c1cc(CS(=O)(=O)N(CC)CC)nc(-c2ccc3[nH]c(CNC)cc3n2)n1]

¹H NMR (400 MHz, CHLOROFORM-d) δ ppm 0.99 (t, J=7.46 Hz, 3H) 1.14 (t, J=7.09 Hz, 6H) 1.77-2.05 (m, 4H) 2.49 (s, 3H) 3.18-3.26 (m, 4H) 3.34 (td, J=12.90, 3.79 Hz, 1H) 3.58-3.74 (m, 2H) 3.95-4.10 (m, 4H) 4.41 (s, 3H) 5.32 (s, 1H) 6.65 (s, 1H) 6.73 (s, 1H) 7.69 (dd, J=8.44, 0.86 Hz, 1H) 8.27 (d, J=8.56 Hz, 1H). LCMS (System A, UV, ESI): Rₜ=0.95 min, [M+H]⁺ 502.

### Example 31 (S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine

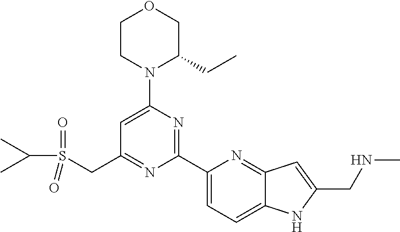 [SMILES: CC[C@H]1COCCN1c1cc(CS(=O)(=O)C(C)C)nc(-c2ccc3[nH]c(CNC)cc3n2)n1]

¹H NMR (400 MHz, METHANOL-d₄) δ ppm 0.98 (t, J=7.34 Hz, 3H) 1.44 (dd, J=6.85, 1.96 Hz, 6H) 1.91 (t, J=7.58 Hz, 2H) 2.24 (s, 1H) 2.46 (s, 3H) 3.37 (s, 3H) 3.42-3.54 (m, 1H) 3.62 (br d, J=2.45 Hz, 1H) 3.70 (dd, J=11.74, 2.93 Hz, 1H) 3.93-4.11 (m, 4H) 4.48 (br d, J=4.40 Hz, 2H) 6.69 (s, 1H) 6.86 (s, 1H) 7.85 (d, J=8.57 Hz, 1H) 8.25 (d, J=8.80 Hz, 1H). LCMS (System A, UV, ESI): Rₜ=0.85 min, [M+H]⁺ 473.

Also prepared was (S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine trifluoroacetate salt

¹H NMR (400 MHz, METHANOL-d₄) δ ppm 1.01 (t, J=7.58 Hz, 3H) 1.49 (d, J=6.85 Hz, 6H) 1.96-2.06 (m, 5H) 2.87 (s, 3H) 3.48 (dt, J=13.69, 6.85 Hz, 2H) 3.59-3.69 (m, 1H) 3.74 (dd, J=12.23, 2.93 Hz, 1H) 3.99-4.11 (m, 2H) 4.54-4.80 (m, 3H) 7.11 (s, 1H) 7.18 (s, 1H) 8.51 (d, J=8.80 Hz, 1H) 8.65 (d, J=8.31 Hz, 1H). LCMS (System A, UV, ESI): Rₜ=0.87 min, [M+H]⁺ 473.

### Example 32 (S)-1-(5-(4-(3-ethylmorpholino)-6-((propylsulfonyl)methyl)pyrimidin-2-yl)-1H-pyrrolo[3,2-b]pyridin-2-yl)-N-methylmethanamine

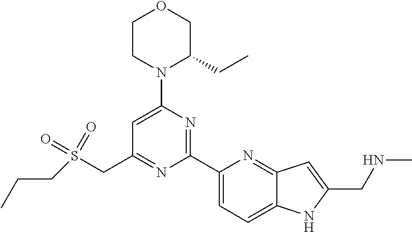 [SMILES: CCCS(=O)(=O)Cc1cc(N2CCOC[C@@H]2CC)nc(-c2ccc3[nH]c(CNC)cc3n2)n1]

¹H NMR (400 MHz, METHANOL-d4) δ ppm 8.30-8.24 (m, 1H), 7.88-7.83 (m, 1H), 6.85 (s, 1H), 6.70 (s, 1H), 4.3-4.6 (m, 4H), 4.05-3.93 (m, 3H), 3.71 (J=11.49, 3.18 Hz, 1H), 3.58-3.67 (m, 1H), 3.23-3.30 (m, 4H), 2.47 (s, 3H), 1.98-1.84 (m, 4H), 1.08 (t, J=7.6 Hz, 3H), 0.99 (t, J=7.6 Hz, 3H). LCMS (System B, UV, ESI): Rₜ=0.43 min, [M+H]⁺ 473.

### Example 33 (S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine

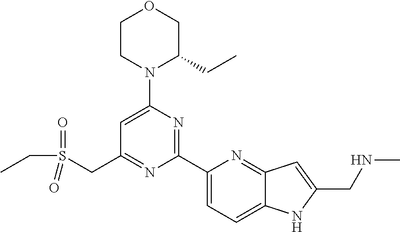 [SMILES: CC[C@H]1COCCN1c1cc(CS(=O)(=O)CC)nc(-c2ccc3[nH]c(CNC)cc3n2)n1]

tert-butyl (S)-((5-(4-(3-ethylmorpholino)-6-((ethylsulfonyl)methyl)pyrimidin-2-yl)-1-((2-(trimethylsilyl)ethoxy)methyl)-1H-pyrrolo[3,2-b]pyridin-2-yl)methyl)(methyl)carbamate (60 mg, 0.087 mmol) was dissolved in TFA and allowed to stand at room temperature overnight, then the solution was evaporated in vacuo to give an orange gum. The crude material was purified by MDAP on high pH method to give (S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine (23 mg, 0.050 mmol, 57.6% yield) as a pale yellow foam. 1H NMR (400 MHz, METHANOL-d4) δ ppm 8.28 (d, J=8.8 Hz, 1H), 7.88-7.83 (m, 1H), 6.85 (s, 1H), 6.69 (s, 1H), 4.55-4.31 (m, 2H), 4.04-3.92 (m, 4H), 3.78-3.53 (m, 2H), 3.37 (s, 2H), 3.30-3.23 (m, 2H), 2.47 (s, 3H), 1.96-1.81 (m, 2H), 1.41 (t, J=7.6 Hz, 3H), 0.98 (t, J=7.3 Hz, 3H).

LCMS (System A, UV, ESI): Rₜ=0.84 min, [M+H]⁺ 459.

Also produced by methods similar to Example 28 was:

### Example 34 (R)-5-ethyl-4-(6-(2-((methylamino)methyl)-1H-pyrrolo[3,2-b]pyridin-5-yl)-4-((methylsulfonyl)methyl)pyridin-2-yl)morpholin-3-one

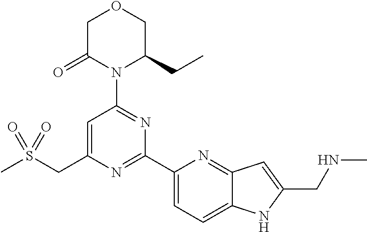

TFA (500 μl, 6.49 mmol) was added to tert-butyl (R)-((5-(6-(3-ethyl-5-oxomorpholino)-4-((methylsulfonyl)methyl)pyridin-2-yl)-1H-pyrrolo[3,2-b]pyridin-2-yl)methyl)(methyl)carbamate

(20.3 mg, 0.036 mmol) in Dichloromethane (DCM) (1000 μl) under nitrogen. The reaction mixture was stirred at RT under nitrogen for 2.5 h.

The reaction mixture was quenched with 5% aqueous potassium carbonate solution (20 ml) and extracted with DCM (4×20 ml). The organic phases were combined, passed through a hydrophobic frit and concentrated under reduced pressure and the residue was purified by Mass Directed Auto-Preparative HPLC (MDAP) on an Xbridge column using acetonitrile in 10 mM ammonium bicarbonate to give

(R)-5-ethyl-4-(6-(2-((methylamino)methyl)-1H-pyrrolo[3,2-b]pyridin-5-yl)-4-((methylsulfonyl)methyl)pyridin-2-yl)morpholin-3-one (11.3 mg, 0.023 mmol, 64.5% yield).

1H NMR (400 MHz, CHLOROFORM-d) δ ppm 0.99 (t, J=7.46 Hz, 3H) 1.71-1.96 (m, 2H) 2.51 (s, 3H) 2.93 (s, 3H) 3.95-4.05 (m, 3H) 4.13-4.24 (m, 2H) 4.28-4.49 (m, 4H) 4.84-4.90 (m, 1H) 6.61 (s, 1H) 7.69 (dd, J=8.56, 0.73 Hz, 1H) 8.06 (d, J=1.22 Hz, 1H) 8.13 (d, J=8.56 Hz, 1H) 8.42 (d, J=1.22 Hz, 1H) 9.15 (br s, 1H)

LCMS (System B, UV, ESI): Rt=0.38 min, [M+H]+ 458.3

### Example 35 Crystal Form A of Example 16

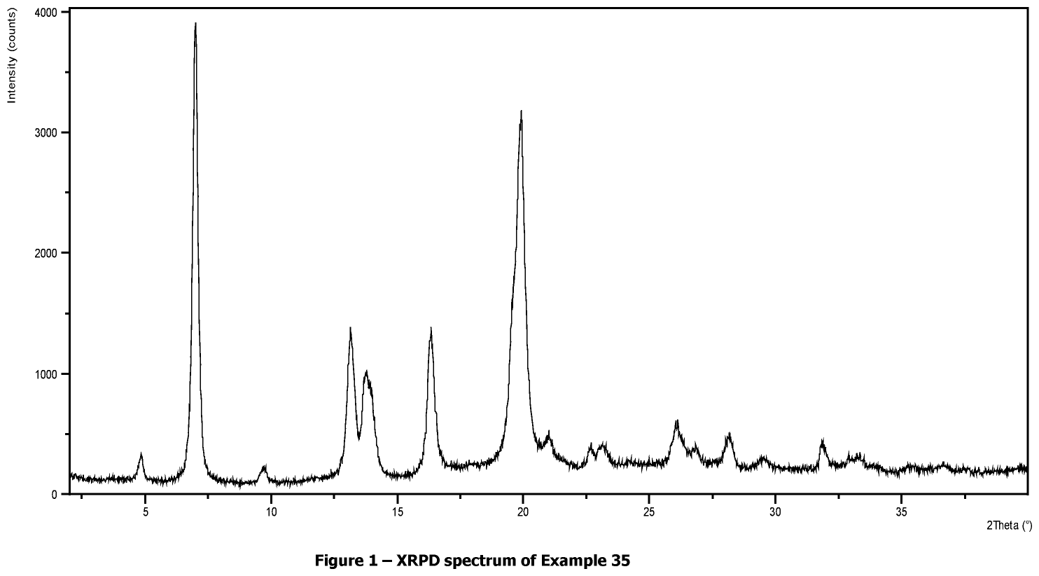

Example 16 (˜300 mg) was weighed into a vial with a stirrer bar. 4.5 mL toluene was added followed by 0.76 mL of a 1M aqueous solution of benzoic acid. The vial was temperature cycled between 0° C. and 40° C. for 4 days. The slurry was filtered, washed with toluene, sucked dry and then dried in vacuo at 40° C. for 3 days. The XRPD spectrum of the compound is shown in FIG. 1.

### Example 36 Crystal Form B of Example 16

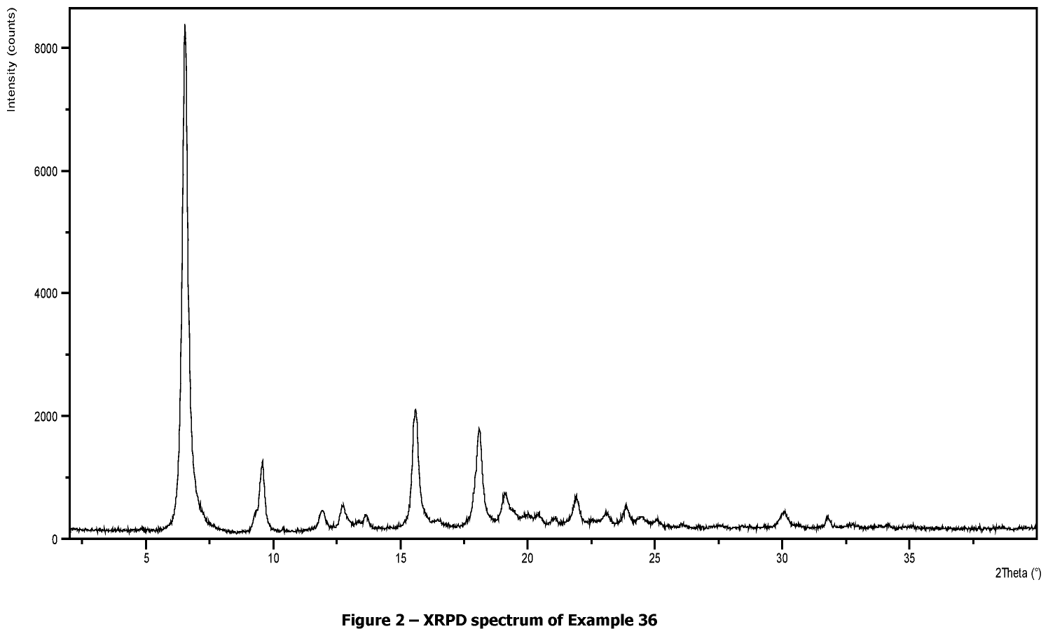

Example 16 (˜300 mg) was weighed into a vial with a stirrer bar. 4.5 mL MeCN was added followed by 0.76 mL of a 1M aqueous solution of benzoic acid. The vial was temperature cycled between 0° C. and 40° C. for 2 days before a further 2 mL MeCN was added and temperature cycling continued for a further 24 h. The slurry was filtered, washed with MeCN, sucked dry and then dried in vacuo at 40° C. for 3 days. The XRPD spectrum of the compound is shown in FIG. 2.

TABLE 1
Characteristic XRPD peak positions and d-spacings for
Example 35 and Example 36

| Example 35 | | Example 36 | |
|---|---|---|---|
| 2θ/° | d-spacings/Å | 2θ/° | d-spacings/Å |
| 4.9 | 18.2 | 6.5 | 13.6 |
| 7.0 | 12.6 | 11.9 | 7.4 |
| 9.8 | 9.1 | 12.8 | 6.9 |
| 13.2 | 6.7 | 15.6 | 5.7 |
| 16.3 | 5.4 | 18.1 | 4.9 |
| 28.2 | 3.2 | 21.9 | 4.1 |
| | | 23.9 | 3.7 |

The data were acquired on a PANalytical X'Pert Pro powder diffractometer, model PW3040/60 using an X'Celerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a silicon wafer (zero background) plate, resulting in a thin layer of powder. The margin of error is approximately f 0.1° 2θ for each of the peak assignments. Peak intensities may vary from sample to sample due to preferred orientation. Peak positions were measured using Highscore software.

### Biological Data

Those of skill in the art will recognise that the assays described below are subject to experimental variability. Accordingly, it is to be understood that the values given below represent the mean of multiple experiments, and that repeating the assay run(s) may result in somewhat different pIC50 values.

### Example 37

The affinity of test compounds for mTOR was determined in the mTOR kinobeads assay. This is a competition-binding assay based on the capturing of endogenously expressed target proteins from cell extracts by a bead-immobilized capturing ligand in the presence of the test compound.

HuT-78 cells (European Collection of Authenticated Cell Cultures, 88041901) were cultured according to vendor's instructions. Frozen cell pellets were homogenized in 3× pellet volumes lysis buffer (50 mM Tris-HCl, 0.4% (v/v) Igepal-CA630, 5% glycerol, 150 mM NaCl, 1.5 mM MgCl₂, 25 mM NaF, 1 mM sodium vanadate, 1 mM DTT, pH 7.5, supplemented with EDTA-free protease inhibitor tablet (Roche)). The sample was dispersed using a Dounce homogenizer, kept rotating for 30 min at 4° C., and centrifuged for 10 min at 20000 gat 4° C. The supernatant was centrifuged again for 1 h at 145 000 g. The protein concentration was determined by Bradford assay (BioRad), aliquots were snap frozen in liquid nitrogen and stored at −80° C.

The capturing matrices were generated by derivatizing N-hydroxysuccinimide (NHS) activated Sepharose 4 beads (GE Healthcare) with the functionalized ligands Compound A and Compound C at a ligand density of 5 mM. Remaining NHS-groups were blocked with ethanolamine.

For the mTOR kinobeads assay the matrices were combined in a 1:1 ratio and equilibrated in DP buffer (50 mM Tris-HCl (pH 7.5), 0.4% (v/v) Igepal-CA630, 5% (v/v) glycerol, 150 mM NaCl, 1.5 mM MgCl₂, 25 mM NaF, 1 mM Na₃VO₄, 1 mM dithiothreitol). All steps of the mTOR kinobeads assay were performed at 4° C. or on ice. The cell lysate was diluted with DP buffer to a final concentration of 5 mg/ml and a final detergent concentration of 0.4% (v/v) Igepal-CA630. For the assay 250 μg cell lysate and 2.5 μl capturing matrix per well (final assay volume: 75 μl) were incubated in the presence of test compounds in a 384-well filter plate (MultiScreenHTS HV Filter Plate, 0.45 μm, MZHVNOW50, Merck Millipore). Each plate contained 16 positive (100 μM Compound B) and 16 negative (2% v/v DMSO) control wells. Compounds were tested in a concentration-response applying 1:3 or 1:4 dilution steps for in total 11 data points. DMSO concentration was 2% (v/v). After 2 h incubation on an overhead shaker (Roto-Shake Genie, Scientific Industries Inc.) at 4° C. the non-bound fraction was removed by washing the beads with DP buffer. Proteins retained on the beads were eluted in SDS sample buffer (200 mM Tris (pH 7.4), 250 mM Trizma Base, 4% (w/v) SDS, 20% (v/v) glycerol, 0.01% (w/v) bromophenol blue, 50 mM dithiothreitol) into a collection plate (384 well polypropylene microplate, V-shape, Greiner, 781 280). Eluates were spotted on nitrocellulose membranes (400 nl per spot) using an automated pin-tool liquid transfer (Biomek FX, Beckman). After drying, the membranes were rehydrated in 20% (v/v) ethanol and blocked by incubation with Odyssey blocking buffer (LICOR, 927-40000) for 1 h at room temperature. Blocked membranes were incubated overnight at 25° C. with Odyssey blocking buffer supplemented with a specific anti-mTOR antibody (Cell Signaling, 2972; 1:500) and 0.4% TWEEN-20. Then the membranes were washed in PBST buffer and incubated for 60 minutes at room temperature with the detection antibody (IRDye™ labelled antibody from LI-COR) diluted in Odyssey blocking buffer (LICOR 927-40000) containing 0.2% TWEEN-20. Then the membranes were washed with PBST and finally rinsed twice with PBS buffer to remove residual Tween-20. The membranes were then scanned with the Odyssey® Infrared Imaging System (LI-COR Biosciences). Fluorescence signals were recorded and analyzed according to the instructions of the manufacturer. Concentration response curves were computed with the software Activity Base. All data were normalized to the mean of 16 high (negative control) and 16 low (positive control) control wells on each plate. Concentration-response curves were fitted using a 4 parameter logistic fit using the equation: Y=A+(B−A)/(1+10^{LogIC50-X*D); where: Y=response, A=minimum response (positive control), B=maximum response (negative control), D=slope factor, x=log(Molar compound concentration). The pIC}₅₀ values are the negative logarithm of the IC₅₀ value.

Compounds of the Examples 1 to 29 were tested in the above assay and had mean pIC₅₀ values greater than 6.9. Compounds of the Examples 1 to 18 and 21 to 29 had mean pIC₅₀ values of 7.9 or greater. Compounds of Examples 1, 2, 4 to 6, 9 to 16, 23 to 27, and 29 had mean pIC₅₀ values of 8.9 or greater. Compounds of Examples 5, 9 to 12, 14, 15, and 26, had mean pIC₅₀ values of 9.5 or greater. Examples 5, 10, 11, and 14, had a mean pIC₅₀ values of 9.7 or greater and Example 10 had a mean pIC₅₀ value of 10.1 or greater. Example 34 had a mean pIC₅₀ value of 5.9

TABLE 1
Selected compounds with their mean pIC₅₀ values when tested
in the mTOR binding assay.

| Ex | Compound | mTOR |
|---|---|---|
| 1 | 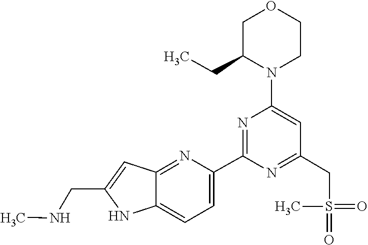 [SMILES: CC[C@H]1COCCN1c1cc(CS(C)(=O)=O)nc(-c2ccc3[nH]c(CNC)cc3n2)n1] | 9.1 |
| | [(5-{4-[(3S)-3-ethylmorpholin-4-yl]-6- | |
| | (methanesulfonylmethyl)pyrimidin-2-yl}-1H-pyrrolo | |
| | [3,2-b]pyridin-2-yl)methyl](methyl)amine | |
| 14 | 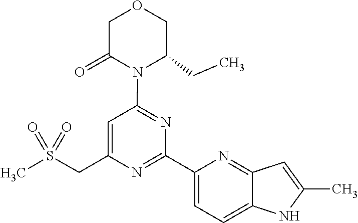 | 9.7 |
| | (5S)-5-ethyl-4-[6-(methanesulfonylmethyl)-2-{2-methyl-1H- | |
| | pyrrolo[3,2-b]pyridin-5-yl}pyrimidin-4-yl]morpholin-3-one | |
| 15 | 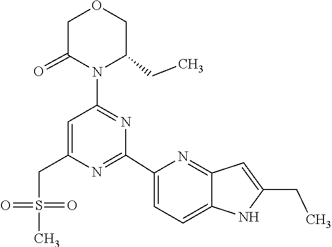 [SMILES: CCc1cc2nc(-c3nc(CS(C)(=O)=O)cc(N4C(=O)COC[C@@H]4CC)n3)ccc2[nH]1] | 9.5 |
| | [(5-{4-[(3S)-3-ethylmorpholin-4-yl]-6- | |
| | (4-methanesulfonyloxan-4-yl)pyrimidin- | |
| | 2-yl}-1H-pyrrolo[3,2-b]pyridin- | |
| | 2-yl)methyl](methyl)amine | |
| 16 | 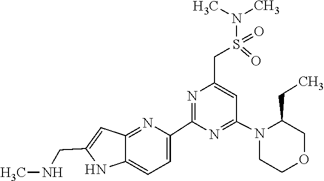 [SMILES: CC[C@H]1COCCN1c1cc(CS(=O)(=O)N(C)C)nc(-c2ccc3[nH]c(CNC)cc3n2)n1] | 9.2 |
| | 1-{6-[(3S)-3-ethylmorpholin-4-yl]-2-{2- | |
| | [(methylamino)methyl]-1H-pyrrolo[3,2-b]pyridin-5- | |
| | yl}pyrimidin-4-yl}-N,N-dimethylmethanesulfonamide | |
| 18 | 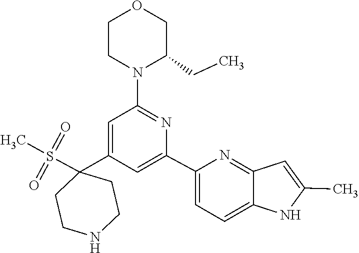 | 8.2 |
| | (S)-3-ethyl-4-(6-(2-methyl-1H-pyrrolo[3,2-b]pyridin-5-yl)-4- | |
| | (4-(methylsulfonyl)piperidin-4-yl)pyridin-2-yl)morpholine | |
| 24 | 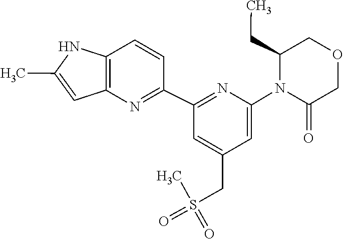 [SMILES: CC[C@H]1COCC(=O)N1c1cc(CS(C)(=O)=O)cc(-c2ccc3[nH]c(C)cc3n2)n1] | 9.3 |
| | (S)-5-ethyl-4-(6-(2-methyl-1H-pyrrolo[3,2-b] | |
| | pyridin-5-yl)-4-((methylsulfonyl) | |
| | methyl)pyridin-2-yl)morpholin-3-one | |

Compound Structures

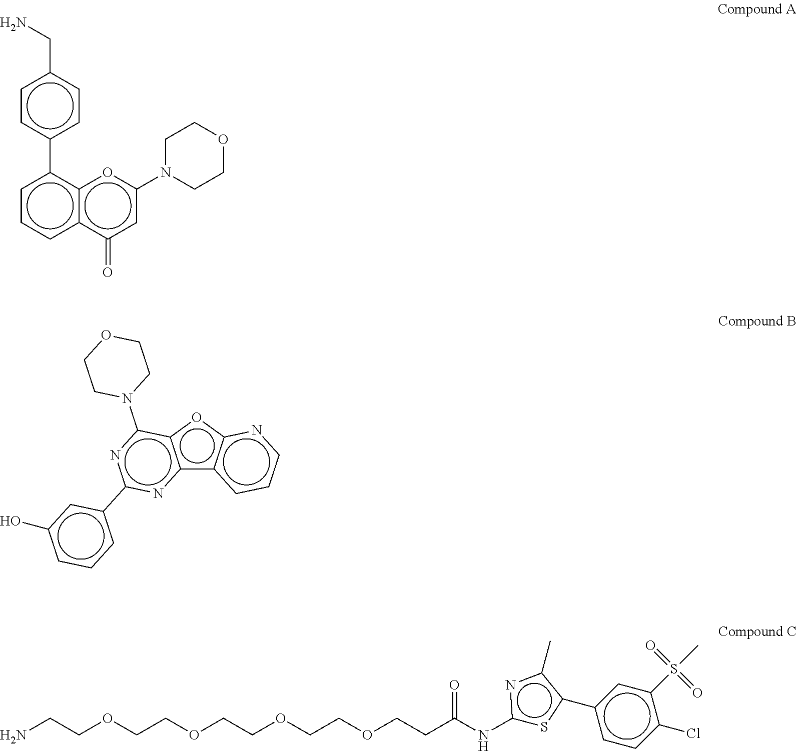

### Example 38 High Throughput Phosphorylated Akt (Ser473) In Vitro Immunoassay

Immunoassay to measure effect of test compounds on phosphorylated Akt at serine 473 (pAKTs473) in primary human lung fibroblasts (Lonza Group Ltd, Basel, Switzerland, Catalogue No. CC-2512) using the MesoScale Discovery (MSD) platform to quantify levels of total Akt and pAKTser473.

Human lung fibroblasts were routinely maintained in fibroblast basal medium supplemented with 2% foetal bovine serum (FBS), 0.1% human fibroblast growth factor (FGF)-B, 0.1% insulin, 0.1% GA-100 (Lonza Group Ltd, Basel, Switzerland, Catalogue No. CC-3132) at 37° C. in 5% CO₂ according to the manufacturer's protocol.

For the assay, cells were harvested using Trypsin/EDTA, (Lonza Group Ltd, Basel, Switzerland, Catalogue No. CC-5012), at working concentration of 0.025%, resuspended to give 3×10⁵ cells/ml and 50 ul/well seeded into 384-well plates (Greiner Group, Kremsmunster, Austria, Catalogue No. 781091) in media containing 0.4% FBS and incubated o/n at 37° C. in 5% CO₂.

Test compounds were dissolved in 100% DMSO to give 10 mM stock solutions and serially diluted to generate an 11-point concentration response curve. Compound were further diluted by 500-fold into 384-well V-bottom polypropylene plates (Greiner Group, Kremsmunster, Austria, Catalogue No. 781280) with media containing 0.4% FBS to give 50 M top compound concentration in 0.5% DMSO.

Compounds were transferred to 384-well plates contacting cells (0.1% DMSO final) and incubated for 1 h at 37° C. in 5% CO₂, prior to stimulation with platelet-derived growth factor (PDGF)-BB (R&D Systems, Catalogue No. 220-BB) at 10 ng/ml (final) for 10 min at RT. Cell assay plates were incubated on ice and treated with lysis buffer (Cell Signalling Technology, Catalogue No. 9806) containing protease and phosphatase inhibitors (Thermo Scientific, Catalogue No. 78444) followed by shaking for 30 min at 4° C.

MSD plates (MSD, MA6000 384-well GAR plate, Catalogue No. L21RA-2) were blocked with blocking buffer containing TBS (MSD, Cat. No. R₆₁TX-1) with 0.5% bovine serum albumin (BSA) (Sigma Aldrich, Cat. No. A7906), 0.1% tween-20 (Sigma Aldrich, Catalogue No. P2287) and coated with rabbit-anti-human pAkt (s473) ab (Cell Signalling Technology, Catalogue No. CST #4060) at RT with shaking, followed by washing (×3) with 1×TBS wash reagent containing 0.1% Tween-20. Cell lysates (30 ul) were added and MSD plates centrifuged at 1000 rpm at 4° C. o/n.

Plates were washed with 1×TBS wash buffer (×3), prior to addition of mouse-anti-human-total Akt ab (1 mg/ml) (Upstate (Milipore), Catalogue No. 05-591) in blocking buffer and shaken at RT for 1 h. After washing with 1×TBS wash buffer (×3), plates were incubated with goat-anti-mouse SULFO-TAG detection ab (MSD, Catalogue No. R₃₂AC-1) (1:500 in blocking buffer) for 1 h at RT. Plates were washed with 1×TBS (×3) and read buffer (2×) (MSD, Catalogue No. R₉₂TC-1) added before detecting electrochemiluminescence (MSD Sector Imager 6000).

Data analysis was performed by determining % inhibition values for test compounds relative to the minimum (+PDGF-BB stimulation) and maximum responses (no PDGF-BB stimulation) with non-linear regression analysis to determine IC₅₀ values for test compounds.

Compounds of the Examples 1 to 29 (apart from the Compounds of Examples 11 and 12) were tested in the above assay, and had mean pIC₅₀ values of greater than 6.3. Compounds of the Examples, had mean pIC₅₀ values of 6.3 to 8.6. Compounds of Examples 1, 8, 9, 10, 13, 14, 15, 16, 21, 22, 23, 24, 25, and 26 had mean pIC₅₀ values of 7.6 to 8.6. Example 14 had a mean pIC₅₀ value of 8.5, and Example 21 had a mean pIC₅₀ value of 8.6. Example 34 had a mean pIC₅₀ value of 5.1.

### Example 39 Phosphorylated Akt (Ser473) In Vitro Immunoassay

Immunoassay using the Meso Scale Discovery (MSD) platform to measure the effect of test compounds on phosphorylated Akt at serine 473 (pAKTs473) in primary lung fibroblasts derived from explanted tissue. The human biological samples were sourced ethically and their research use was in accord with the terms of the informed consents under an IRB/EC approved protocol.

Human lung fibroblasts were routinely maintained in Dulbecco's Modified Eagle Medium (DMEM; Gibco, catalogue no. 21969-035) supplemented with 10% foetal bovine serum (FBS (heat-inactivated); Gibco, catalogue no. 10270-106) and 4 mM L-glutamine (Gibco, catalogue no. 25030024) at 37° C. in 10% CO₂.

For the assay, cells were harvested using Trypsin/EDTA (Gibco, catalogue no. 25300-062), resuspended to give 1.2×10⁵ cells/ml and 100 ul/well seeded into 96-well plates (Corning, Catalogue No. 3596) in DMEM supplemented with 0.4% FCS and 4 mM L-glutamine and incubated overnight at 37° C. in 10% CO₂.

Test compounds were dissolved in 100% DMSO to give 10 mM stock solutions and serially diluted to generate a 10-point concentration response curve. Compounds were further diluted 100-fold into 96-well V-bottom polypropylene plates (Greiner, Catalogue no. 651201) with DMEM supplemented with 0.4% FCS and 4 mM L-glutamine to give 100 M top compound concentration in 1% DMSO.

Compounds were further diluted 10-fold by transfer to 96-well plates containing cells (10 M top concentration, 0.1% DMSO final) and incubated for 2 h at 37° C. in 10% CO₂, prior to stimulation with platelet-derived growth factor (PDGF)-BB (Gibco, Catalogue no. PHG0041) at 10 ng/ml (final) for 15 min at 37° C. in 10% CO₂. Cell assay plates were then incubated on ice and treated with lysis buffer (Cell Signalling Technology, Catalogue No. 9803) containing protease and phosphatase inhibitors (Halt, Thermo Scientific, Catalogue No. 78444) for 30 min.

MSD plates (MSD, Phospho(Ser473)/Total Akt Whole Cell Lysate Kit, Catalogue No. K15100D-3) were blocked with 1×blocking buffer as per the manufacturer's instructions at RT with shaking, followed by washing (×3) with Tris-buffered saline containing 0.05% Tween-20 (TBST). Cell lysates (30 ul) were added and MSD plates incubated for 1 h at RT with shaking.

Plates were washed with TBST (×3), prior to addition of SULFO-TAG Anti-Total Akt Antibody as per the manufacturer's instructions and shaken at RT for 1 h. After washing with 1×TBST (×3), read buffer (1×) (MSD, Catalogue No. R₉₂TC-1) was added as per the manufacturer's instructions before detecting electrochemiluminescence (MSD Sector Imager 600).

Data analysis was performed using non-linear regression analysis to determine IC₅₀ values for test compounds. Minimum (no PDGF-BB stimulation) and maximum responses (+PDGF-BB stimulation) were monitored for assay quality control.

Compounds of the Examples 1, 6, 14, 15, 16, 18, 21, 23, 24, 30, 31 and 32 were tested in the above assay, and had a mean pIC₅₀ value of greater than 7. Compounds of Examples 14, 16, 18, 21, 23, 30 and 31 had mean pIC₅₀ values of greater than 8.

### Example 40 High Throughput Type I Collagen Deposition (Scar-In-a-Jar) High Content Screening Assay

Highthroughput, cellular type I collagen deposition was measured by adapting a published method; scar-in-a-jar (Chen et al., 2009) to primary lung fibroblasts derived from human tissue and combining with high content analysis to permit fluorescent quantification of collagen deposited in the extracellular matrix (ECM). The human biological samples were sourced ethically and their research use was in accordance with the terms of the informed consents.

Test compounds were dissolved in 100% DMSO to give 10 mM (stocks) and further diluted to give half log dilutions to generate an 11 point concentration response curve. 1 ul was transferred to 384-well plates (Greiner Group, Kremsmunster, Austria, Catalogue No Greine 781280r).

Human pulmonary fibroblasts were maintained in DMEM media (Gibco, Catalogue No 21969) supplemented with 4 mM L-glutamine (Gibco Catalogue No 25030-024) and 10% heat inactivated FBS (Gibco, Catalogue No 10099-141) at 37° C. in 10% CO₂. Cells were harvested and resuspended at 4×10⁶ cells/ml in a T175 cell culture flask (BD Falcom, Catalogue No: 353112) containing 50 mL of culture media. After 4 days 37° C. in 10% CO₂, cells were harvested and seeded at 4000 cells per well (50 L/well) into 384-well plates (BD Falcon, Catalogue No 353962Greiner) in assay media (0.4% heat inactivated FBS, 4 mM L-glutamine). Plates were sealed with breathable seals (Sigma, Catalogue No. BEM-1). Assay plates were incubated for 72 h at 37° C. in 10% CO₂.

Assay media was supplemented with 112.5 mg/ml Ficoll 70 (Sigma, Catalogue No F2878), 75 mg/ml Ficoll 400 (Sigma, Catalogue No F4375) and 50 g/ml ascorbic acid (Sigma, Catalogue No A8960). 30 μL assay media were added to 1 l compound (37 M compound concentration in 1% DMSO). Diluted compounds (10 μL) were transferred to plates containing cells and incubated for 3 h at 37° C. in 10% CO₂. Transforming growth factor (TGF)-β (R&D systems, Catalogue No 100-B/CF) was reconstituted to give 10 g/mL (stock) and further diluted in assay media containing Ficoll and ascorbic acid (1 in 3333). TGF-β was added to cell plates (1 ng/ml final) and cells incubated for 72 h at 37° C. in 10% CO₂.

Media was aspirated from cells and 30 L/well 100% ice-cold methanol added and incubated at RT for 5 min to fix cells. Methanol was aspirated and cells washed with PBS (3×). To block and permeabilise, cells were incubated with PBS containing 1% BSA and 0.1% Triton X 100 for 20 min, followed by PBS wash (3×). Cells were incubated with mouse anti human type I collagen monoclonal antibody (Sigma, Catalogue No C₂₄₅₆) diluted 1:1000 in PBS for 24 h at 4° C. Cells were washed with PBS containing 0.1% Tween-20 (3×) and incubated with secondary alexa fluor 488 goat-anti-mouse antibody (Invitrogen, Catalogue No A11001, 1 in 500 in PBS) and Hoechst 33342 (Sigma, Catalogue No H21492), 1 in 1000 in PBS) for 1 h at RT, prior to washing with PBS containing 0.1% Tween (×4).

Type I collagen immunoreactivity was detected using the INCell 2000 Analyzer (GE Healthcare, INcell 2000) set to excitation and emission wavelengths for DAPI and FITC with laser focus and exposure adjusted to appropriate levels. Images were imported and analysed using Columbus software (Perkin Elmer) and computational algorithms to quantify “total collagen area” and “cell count”.

Data analysis was performed by determining 0/inhibition values for test compounds relative to the minimum (media only) and maximum responses (TGF-βstimulation) with non-linear regression analysis to determine IC₅₀ values for test compounds.

Compounds of the Examples 1 to 29 (apart from Compound of Example 3), were tested in the above assay, and had mean PIC₅₀ values of greater than 5.5. Compounds of Examples 1, 2, and 4 to 29 had mean pIC₅₀ values of 5.5 to 7.7. Compounds of Examples 1, 8 to 16, 18, and 21 to 26 had mean pIC₅₀ values of 6.5 to 7.7. The compound of Example 11 had a mean pIC₅₀ value of 7.7.

TABLE 2
Selected compounds with their mean pIC₅₀ values
when tested in the scar-in-a-jar assay.

| | | Scar |
|---|---|---|
| Ex | Compound | in a jar |
| 1 | 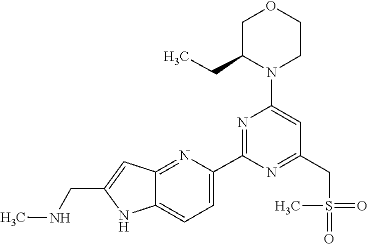 [SMILES: CC[C@H]1COCCN1c1cc(CS(C)(=O)=O)nc(-c2ccc3[nH]c(CNC)cc3n2)n1] | 6.6 |
| | [(5-{4-[(3S)-3-ethylmorpholin-4-yl]-6- | |
| | (methanesulfonylmethyl)pyrimidin-2-yl}-1H-pyrrolo | |
| | [3,2-b]pyridin-2-yl)methyl](methyl)amine | |
| 14 | 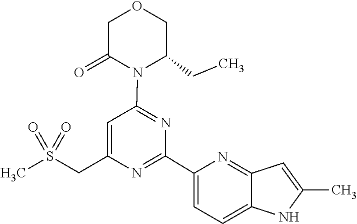 | 7.6 |
| | (5S)-5-ethyl-4-[6-(methanesulfonylmethyl)-2- | |
| | {2-methyl-1H-pyrrolo[3,2-b]pyridin-5- | |
| | yl}pyrimidin-4-yl]morpholin-3-one | |
| 15 | 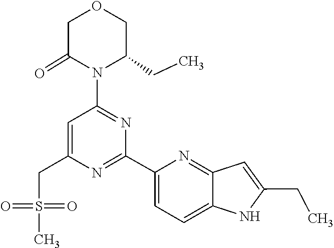 [SMILES: CCc1cc2nc(-c3nc(CS(C)(=O)=O)cc(N4C(=O)COC[C@@H]4CC)n3)ccc2[nH]1] | 6.8 |
| | [(5-{4-[(3S)-3-ethylmorpholin-4-yl]-6- | |
| | (4-methanesulfonyloxan-4-yl)pyrimidin-2-yl}-1H-pyrrolo | |
| | [3,2-b]pyridin-2-yl)methyl](methyl)amine | |
| 16 | 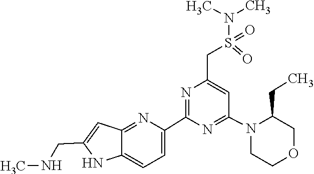 [SMILES: CC[C@H]1COCCN1c1cc(CS(=O)(=O)N(C)C)nc(-c2ccc3[nH]c(CNC)cc3n2)n1] | 7 |
| | 1-{6-[(3S)-3-ethylmorpholin-4-yl]-2-{2- | |
| | [(methylamino)methyl]-1H-pyrrolo[3,2-b] | |
| | pyridin-5-yl}pyrimidin-4-yl}-N,N- | |
| | dimethylmethanesulfonamide | |
| 18 | 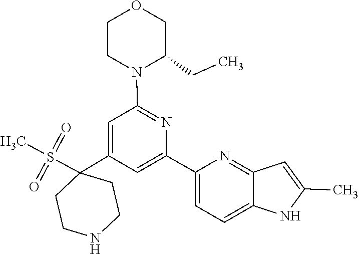 | 6.8 |
| | (S)-3-ethyl-4-(6-(2-methyl-1H-pyrrolo[3,2-b] | |
| | pyridin-5-yl)-4-(4-(methylsulfonyl) | |
| | piperidin-4-yl)pyridin-2-yl)morpholine | |
| 24 | 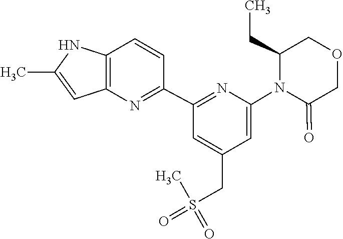 [SMILES: CC[C@H]1COCC(=O)N1c1cc(CS(C)(=O)=O)cc(-c2ccc3[nH]c(C)cc3n2)n1] | 6.9 |
| | (S)-5-ethyl-4-(6-(2-methyl-1H-pyrrolo | |
| | [3,2-b]pyridin-5-yl)-4-((methylsulfonyl) | |
| | methyl)pyridin-2-yl)morpholin-3-one | |

### Example 41 Type I Collagen Deposition (Scar-In-a-Jar) High Content Screening Assay

Cellular type I collagen deposition was measured by adapting a published method; scar-in-a-jar (Chen et al., 2009) to primary lung fibroblasts derived from human tissue and combining with high content analysis to permit fluorescent quantification of collagen deposited in the extracellular matrix (ECM). The human biological samples were sourced ethically and their research use was in accord with the terms of the informed consents under an IRB/EC approved protocol. Human pulmonary fibroblasts were maintained in DMEM media (Gibco, Catalogue No 21969) supplemented with 4 mM L-glutamine (Gibco Catalogue No 25030-024) and 100/heat inactivated FBS (Gibco #10270-106, Lot #42F6663K) at 37° C. in 10%/CO₂. Cells were harvested and seeded at 10,000 cells per well (85 μL/well) into black walled 96 well imaging plates (BD Falcon, 353219) in assay media (0.4%/heat inactivated FBS, 4 mM L-glutamine) and incubated overnight at 37° C., 10%/CO₂.

Test compounds were dissolved in 1000/DMSO to give 10 mM stocks which were further diluted (serial 1:3) to generate 10 point concentration response curves.
Assay media was supplemented with 112.5 mg/ml Ficoll 70 (Sigma, Catalogue No F2878), 75 mg/ml Ficoll 400 (Sigma, Catalogue No F4375) and 50 g/ml ascorbic acid (Sigma, Catalogue No A8960). 3 μL compound were added to 297 μL assay media (10× compound final concentration in 1% DMSO).

Diluted compounds (15 μL) were transferred to plates containing cells and incubated for 3 h at 37° C. in 10% CO₂. Transforming growth factor (TGF)β-1 (R&D Systems, Catalogue No 100-B/CF) was reconstituted according to manufacturer's instructions to give 10 g/mL (stock) and further diluted (1 in 3333) in assay media containing Ficoll and ascorbic acid. 50 μL TGFβ-1 was added to cell plates (1 ng/ml final) and cells incubated for 72 h at 37° C. in 10% CO₂.

Media was removed and cells were fixed with 50 L/well 100% ice-cold methanol (2 min). Methanol was decanted and cells washed with PBS (3×). To permeabilise, cells were incubated with PBS containing 0.1% Triton X 100 for 90 sec, followed by PBS wash (3×). Cells were incubated with mouse anti human type I collagen monoclonal antibody (Sigma, Catalogue No C2456) diluted 1:1000 in PBS overnight at 4° C. Cells were washed with PBS containing 0.05% Tween-20 (3×) and incubated with secondary alexa fluor 488 goat-anti-mouse antibody (Invitrogen, Catalogue No A11001, 1 in 500 in PBS) and Hoechst 33342 (Sigma, Catalogue No H21492), 1 in 10000 in PBS) for 1 h at RT, prior to washing with PBS containing 0.05% Tween (×3) before finally adding 200 L/well PBS.

Type I collagen immunoreactivity was detected using the Thermofisher CellInsight NXT set to excitation and emission wavelengths for DAPI and FITC with exposure adjusted to appropriate levels. Images were analysed using Cellomics Studio software (Version 6.6.0, build 8153) and computational algorithms to quantify “collagen intensity” and “cell count”.

Data analysis was performed by determining % response values for test compounds relative to the maximum (TGFβ-1 stimulation) fitting a ‘log(inhibitor) vs. response-Variable slope (four parameters)’ curve and recording pIC50 (inverse of Log IC50) values for test compounds.

Compounds of the Examples 2, 8, 14, 15, 16, 18, 21, 24, 27, 30, 31, 32, and 33 were tested in the above assay, and had a mean pIC₅₀ value of greater than 5.4. Compounds of the Examples 4 & 6 were tested in the above assay, and had a mean pIC₅₀ value of less than 5.4. Compounds of Examples 14, 15, 16, 21, 24 & 31 had mean pIC₅₀ values of 6.6 or greater. The compound of Example 16 has a mean pIC₅₀ value of 6.6.

1. A compound of formula (I) 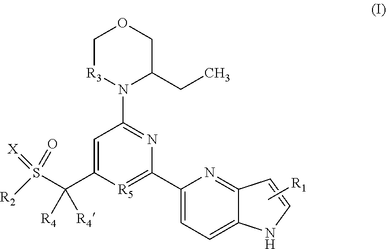
wherein:
X is O or NH;
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂;
R₃ is CH₂ or C═O;
R₄ and R₄′ are both H, or R₄ and R₄′ combine to form a 5- or 6-membered heterocycloalkylene which is unsubstituted or substituted with (C₁-C₃)alkyl; and
R₅ is CH or N;
wherein when R₅ is CH and R₁ is (C₂-C₃)alkyl, CH₂NH(C₁-C₃)alkyl, or CH₂OH, then R₃ is not CH₂;
or a pharmaceutically acceptable salt thereof.

2. A compound according to claim 1, wherein the compound is of formula (II) 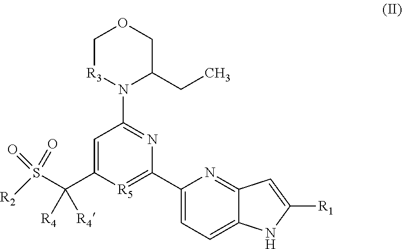
wherein:
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂;
R₃ is CH₂ or C═O;
R₄ and R₄′ are both H, or R₄ and R₄′ combine to form a 5- or 6-membered heterocycloalkylene which is unsubstituted or substituted with (C₁-C₃)alkyl; and
R₅ is CH or N;
wherein when R₅ is CH and R₁ is (C₂-C₃)alkyl, CH₂NH(C₁-C₃)alkyl, or CH₃₀H, then R₃ is not CH₂;
or a pharmaceutically acceptable salt thereof.

3. A compound or pharmaceutically acceptable salt thereof according to claim 2, of formula (IIa) 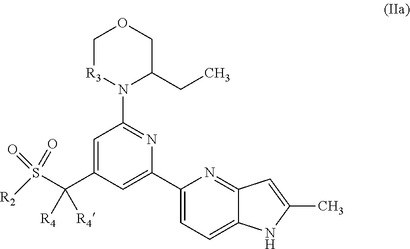
wherein:
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or of formula (IIb) 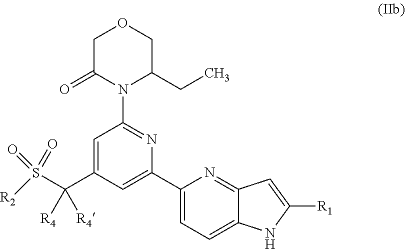
wherein:
R₁ is (C₁-C₃)alkyl, CH₂NH(C₁-C₃)alkyl, or (C₁-C₃)alkyl-OH;
R₂ is methyl, N(H)CH₃, N(CH₃)₂, or NH₂; and
R₄ and R₄′ are both H, or R₄ and R₄′ combine to form a 5- or 6-membered heterocycloalkylene which is unsubstituted or substituted with (C₁-C₃)alkyl;
or of formula (IIc) 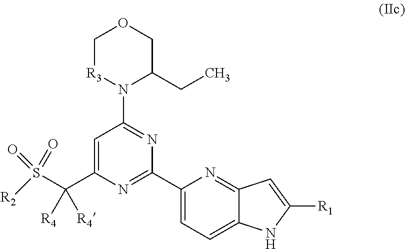
wherein:
R₁ is (C₁-C₃)alkyl, CH₂NH(C₁-C₃)alkyl, or (C₁-C₃)alkyl-OH;
R₂ is methyl, N(H)CH₃, N(CH₃)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl.

4. A compound according to claim 3, of formula (IIa) 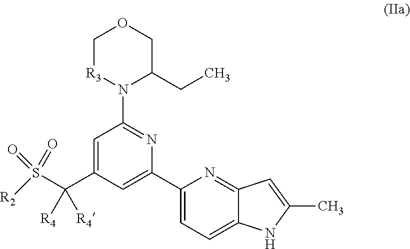
wherein:
R₂ is (C₁-C₃)alkyl, N(H)(C₁-C₃)alkyl, N((C₁-C₃)alkyl)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof.

5. A compound according to claim 3, of formula (IIb) 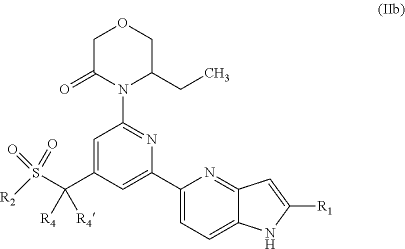
wherein:
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof.

6. A compound according to claim 3, of formula (IIc) 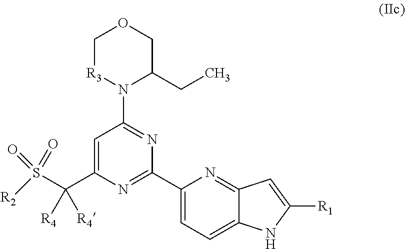
wherein:
R₁ is (C₁-C₃)alkyl, CH₂NH(C₁-C₃)alkyl, or (C₁-C₃)alkyl-OH;
R₂ is (C₁-C₃)alkyl, N(H)(C₁-C₃)alkyl, N((C₁-C₃)alkyl)₂, or NH₂;
R₃ is CH₂ or C═O; and
R₄ and R₄′ are both H, or R₄ and R₄′ combine to form a 5- or 6-membered heterocycloalkylene which is unsubstituted or substituted with (C₁-C₃)alkyl;
or a pharmaceutically acceptable salt thereof.

7. A compound according to claim 6, of formula (III) 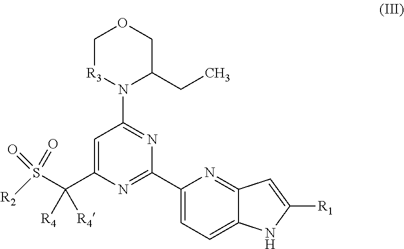
wherein:
R₁ is CH₂NH(C₁-C₃)alkyl;
R₂ is isopropyl, or NH₂;
R₃ is CH₂; and
R₄ and R₄′ are both H;
or a pharmaceutically acceptable salt thereof.

8. A compound or pharmaceutically acceptable salt according to claim 7, wherein the ethyl substituent on the morpholine ring is in the the S-diastereoisomer:

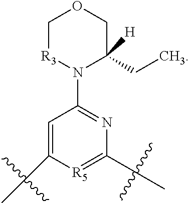

9. A compound according to claim 1, which is:
[(5-{4-[(3S)-3-ethylmorpholin-4-yl]-6-(methanesulfonylmethyl)pyrimidin-2-yl}-1H-pyrrolo[3,2-b]pyridin-2-yl)methyl](methyl)amine;
(S)-(5-(4-(3-ethylmorpholino)-6-((methylsulfonyl)methyl)pyrimidin-2-yl)-1H-pyrrolo[3,2-b]pyridin-2-yl)methanol;
[(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methyl](methyl)amine;
(5-{4-[(3S)-3-ethylmorpholin-4-yl]-6-(4-methanesulfonyloxan-4-yl)pyrimidin-2-yl}-1H-pyrrolo[3,2-b]pyridin-2-yl)methanol;
(5S)-5-ethyl-4-{2-[2-(hydroxymethyl)-1H-pyrrolo[3,2-b]pyridin-5-yl]-6-(methanesulfonylmethyl)pyrimidin-4-yl}morpholin-3-one;
{6-[(3S)-3-ethylmorpholin-4-yl]-2-{2-[(methylamino)methyl]-1H-pyrrolo[3,2-b]pyridin-5-yl}pyrimidin-4-yl}methanesulfonamide;
(S)-1-(2-(3-ethyl-5-oxomorpholino)-6-(2-((methylamino)methyl)-1H-pyrrolo[3,2-b]pyridin-5-yl)pyridin-4-yl)-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N,N-dimethylmethanesulfonamide;
1-{6-[(3S)-3-ethylmorpholin-4-yl]-2-[2-(hydroxymethyl)-1H-pyrrolo[3,2-b]pyridin-5-yl]pyrimidin-4-yl}-N-methylmethanesulfonamide;
1-{6-[(3S)-3-ethylmorpholin-4-yl]-2-{2-methyl-1H-pyrrolo[3,2-b]pyridin-5-yl}pyrimidin-4-yl}-N-methylmethanesulfonamide;
(3S)-3-ethyl-4-[6-(methanesulfonylmethyl)-2-{2-methyl-1H-pyrrolo[3,2-b]pyridin-5-yl}pyrimidin-4-yl]morpholine;
(3S)-3-ethyl-4-[6-(4-methanesulfonyloxan-4-yl)-2-{2-methyl-1H-pyrrolo[3,2-b]pyridin-5-yl}pyrimidin-4-yl]morpholine;
(5S)-5-ethyl-4-[6-(methanesulfonylmethyl)-2-{2-methyl-1H-pyrrolo[3,2-b]pyridin-5-yl}pyrimidin-4-yl]morpholin-3-one;
(5S)-5-ethyl-4-(2-{2-ethyl-1H-pyrrolo[3,2-b]pyridin-5-yl}-6-(methanesulfonylmethyl)pyrimidin-4-yl)morpholin-3-one;
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(3S)-3-ethyl-4-[6-(4-methanesulfonylpiperidin-4-yl)-2-{2-methyl-1H-pyrrolo[3,2-b]pyridin-5-yl}pyrimidin-4-yl]morpholine;
(S)-3-ethyl-4-(6-(2-methyl-1H-pyrrolo[3,2-b]pyridin-5-yl)-4-(4-(methylsulfonyl)piperidin-4-yl)pyridin-2-yl)morpholine;
(S)-3-ethyl-4-(6-(2-methyl-1H-pyrrolo[3,2-b]pyridin-5-yl)-4-(1-methyl-4-(methylsulfonyl)piperidin-4-yl)pyridin-2-yl)morpholine;
(S)-(5-(6-(3-ethylmorpholino)-4-(1-methyl-4-(methylsulfonyl)piperidin-4-yl)pyridin-2-yl)-1H-pyrrolo[3,2-b]pyridin-2-yl)methanol;
(S)-3-ethyl-4-(6-(2-methyl-1H-pyrrolo[3,2-b]pyridin-5-yl)-4-(4-(methylsulfonyl)tetra-hydro-2H-pyran-4-yl)pyridin-2-yl)morpholine;
(S)-3-ethyl-4-(6-(2-methyl-1H-pyrrolo[3,2-b]pyridin-5-yl)-4-((methylsulfonyl)methyl) pyridin-2-yl)morpholine;
(S)-(2-(3-ethylmorpholino)-6-(2-methyl-1H-pyrrolo[3,2-b]pyridin-5-yl)pyridin-4-yl)methanesulfonamide;
(S)-5-ethyl-4-(6-(2-methyl-1H-pyrrolo[3,2-b]pyridin-5-yl)-4-((methylsulfonyl) methyl)pyridin-2-yl)morpholin-3-one;
(S)-5-ethyl-4-(6-(2-methyl-1H-pyrrolo[3,2-b]pyridin-5-yl)-4-(4-(methylsulfonyl) tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-(4-(methylsulfonyl)tetrahydro-2H-pyran-4-yl)pyridin-2-yl)morpholin-3-one;
(S)-5-ethyl-4-(6-(2-(hydroxymethyl)-1H-pyrrolo[3,2-b]pyridin-5-yl)-4-((methylsulfonyl) ethyl)pyridin-2-yl)morpholin-3-one;
(S)-5-ethyl-4-(6-(2-((methylamino)methyl)-1H-pyrrolo[3,2-b]pyridin-5-yl)-4-((methylsulfonyl)methyl)pyridin-2-yl)morpholin-3-one;
(S)-1-(2-(3-ethyl-5-oxomorpholino)-6-(2-(hydroxymethyl)-1H-pyrrolo[3,2-b]pyridin-5-yl)pyridin-4-yl)-N,Ndimethylmethanesulfonamide;
or a pharmaceutically acceptable salt thereof.

10. A compound according to claim 1, which is:
(S)—N,N-diethyl-1-(6-(3-ethylmorpholino)-2-(2-((methylamino)methyl)-1H-pyrrolo[3,2-b]pyridin-5-yl)pyrimidin-4-yl)methanesulfonamide;
(S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((propylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine;

11. A compound according to claim 1 which is:
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide;
(S)-1-(5-(4-(3-ethylmorpholino)-6-((isopropylsulfonyl)methyl)pyrimidin-2-yl)-1H-pyrrolo[3,2-b]pyridin-2-yl)-N-methylmethanamine;
(S)-1-(5-(4-(3-ethylmorpholino)-6-((ethylsulfonyl)methyl)pyrimidin-2-yl)-1H-pyrrolo[3,2-b]pyridin-2-yl)-N-methylmethanamine
or a pharmaceutically acceptable salt thereof.

12. A compound according to claim 11 which is
1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide or a pharmaceutically acceptable salt thereof.

13. A compound according to claim 12 which is 1-{6-[(3S)-3-ethylmorpholin-4-yl]-2-{2-[(methylamino)methyl]-1H-pyrrolo[3,2-b]pyridin-5-yl}pyrimidin-4-yl}-N,N-dimethylmethanesulfonamide.

14. A pharmaceutical composition comprising a) a compound or pharmaceutically acceptable salt thereof according to claim 1, and b) a pharmaceutically acceptable excipient.

15. A method for the treatment of disease in which an mTOR kinase inhibitor is indicated in a human in need thereof comprising administering to said human a therapeutically effective amount of a compound or pharmaceutically acceptable salt thereof according to claim 1.

16. A method according to claim 15 wherein the disease is idiopathic pulmonary fibrosis.

17-21. (canceled)
